(19) United States
(12) Patent Application Publication
(10) Pub. No.: US 2025/0122483 A1
(43) Pub. Date: Apr. 17, 2025

(54) Method of Co-Expressing a Sulfatase and a Sulfatase Modifying Factor in a Plant or Plant Cell

(71) Applicant: BIOSTRATEGIES LC, State University, AR (US)

(72) Inventor: David N. RADIN, Jonesboro, AR (US)

(21) Appl. No.: 18/984,148

(22) Filed: Dec. 17, 2024

Related U.S. Application Data
(62) Division of application No. 17/651,106, filed on Feb. 15, 2022, now Pat. No. 12,168,784.
Application No. 17/651,106 is a continuation of application No. 15/325,661, filed on Jan. 11, 2017, now abandoned.
(60) Provisional application No. 62/023,571, filed on Jul. 11, 2014.

(51) Int. Cl. C12N 9/02; A61K 38/16; A61K 38/17; A61K 38/44; C07K 14/42; C12N 9/16; C12N 15/82

(52) U.S. Cl. CPC C12N 9/0051; A61K 38/168; A61K 38/17; A61K 38/44; C07K 14/42; C12N 9/16; C12N 15/8257; C12Y 108/99; C12Y 301/06; C07K 2319/00; C07K 2319/02; C07K 2319/04

(57) ABSTRACT

The subject invention concerns materials and methods for treating or preventing disease and conditions associated with various sulfatase enzymes that are defective or that are not properly expressed in a person or animal. In one embodiment, the disease is Sanfilippo A (MPS-IIIA) disease. The subject invention also concerns materials and methods for treating or preventing multiple sulfatase deficiency (MSD) in a person or animal. Compounds of the invention include a fusion protein comprising i) a mammalian sulfatase, or an enzymatically active fragment or variant thereof, and ii) a plant lectin or a binding subunit thereof. In a specific embodiment, the mammalian sulfatase is a human sulfatase, or an enzymatically active fragment or variant thereof. Polynucleotides encoding the fusion proteins are also contemplated for the subject invention. The subject invention also concerns materials and methods for producing proteins of the invention.

### CROSS-REFERENCE TO RELATED APPLICATIONS

The present application is a division of U.S. application Ser. No. 17/651,106, filed Feb. 15, 2022, now U.S. Patent No. 17/651,106, issued Dec. 17, 2024; which is a continuation of U.S. application Ser. No. 15/325,661, filed Jan. 11, 2017, now abandoned; which is the National Stage of International Application Number PCT/US2015/040030, filed Jul. 10, 2015, which claims the benefit of U.S. Provisional Application Ser. No. 62/023,571, filed Jul. 11, 2014, which is hereby incorporated by reference herein in its entirety, including any figures, tables, nucleic acid sequences, amino acid sequences, or drawings.

### SEQUENCE LISTING

The Sequence Listing for this application is labeled “SeqList-17Dec24.xml” which was created on Dec. 17, 2024 and is 206,275 bytes. The entire contents of the sequence listing is incorporated herein by reference in its entirety.

### BACKGROUND OF THE INVENTION

There is a need in the art for an effective enzyme replacement therapy (ERT) for patients having disorders associated with sulfatase enzymes. For example, Sanfilippo A (mucopolysaccharidosis IIIA; MPS-IIIA) is a rare genetic lysosomal storage disorder (LSD) affecting about 1 in 150,000 births, with prevalence as high as 1/24,000 in some regions. MPS-IIIA is caused by a genetic defect in the gene for the lysosomal enzyme heparan N-sulfatase (N-sulfoglucosamine sulfohydrolase; SGSH) and is characterized by relatively mild somatic features but severe neurological manifestations (decline of learning abilities, hyperactivity, behavior problems, sleep difficulties, seizures) leading to dementia and death during puberty or early adulthood. Currently treatment options are limited to symptom management and development of an effective ERT drug has been hindered by the challenges of severe central nervous system (CNS) involvement in this disease. Humans have multiple sulfatases wherein deficiencies are linked to complex pathologies.

In lysosomal ERT development, the targeting of drug delivery to disease susceptible organs, tissues, cells, and intracellular lysosomes remains challenging. Of the ERTs commercially available for lysosomal disorders, none address neurological pathologies of these diseases. For these ERTs, delivery is based on ERT glycan structure to exploit uptake by high-mannose or mannose-6P receptors. The inventors use genetic engineering to test the potential of fusions of ERT's with non-toxic plant lectin subunits of ricin (RTB) and nigrin (NBB) to facilitate cell uptake and lysosomal delivery. In preliminary studies, it has been demonstrated that RTB a) efficiently carries proteins (>70 kDa) into a broad array of human cells, including brain microvessel endothelial cell layers using mannose/M6P-independent routes, b) transports associated proteins across oral or nasal mucosal surfaces, and c) that RTB:ERT fusions reduce disease substrate levels to normal in lysosomal disease cells including Hurler (MPS I), GM1 gangliosidosis, and Sanfilippo (MPS IIIA) patient fibroblasts. These lectin carriers will provide a fundamental advance in ERTs by improving efficacy through enzyme delivery to a broader array of diseased cells and pathologies and by introducing transmucosal administration strategies to reduce the burden of current patient treatment options.

The promise of plant-made bioproduction systems to effectively meet the stringent manufacture and regulatory criteria for ERT biologics has now been recognized with recent FDA approval of ELELYSO, Protalix/Pfizer's plant-made glucocerebrosidase ERT for Gaucher disease. This plant-based product is less expensive and less susceptible to viral contamination issues that have recently plagued traditional CHO-based manufacture of LSD ERTs. BioStrategies LC founders, Radin and Cramer, pioneered development of plant-based expression of human lysosomal enzymes (U.S. Pat. No. 5,929,304) and continue to develop new technologies to improve production and efficacy of these ERTs. Nevertheless, since plants do not possess the class of mammalian sulfatase related enzymes described in this patent specification, it was not obvious that active forms of these proteins could be successfully expressed in plants.

### BRIEF SUMMARY OF THE INVENTION

The subject invention concerns materials and methods for treating or preventing disease and conditions associated with various sulfatase enzymes that are defective or that are not properly expressed in a person or animal. In one embodiment, the disease is Sanfilippo A (MPS-IIIA) disease. The subject invention also concerns materials and methods for treating or preventing multiple sulfatase deficiency (MSD) in a person or animal. The present invention utilizes the ability of plants to produce bioactive sulfatases and employ a new transient expression system to bring additional advantages of speed and flexible scaled up manufacture that could be particularly well suited for lysosomal stage disorders and other rare disease targets.

Compounds within the scope of the invention include, but are not limited to, a mammalian sulfatase, sulfatase modifying factor (SUMF1), or a fusion protein comprising i) a mammalian sulfatase or SUMF1, or an enzymatically active fragment or variant thereof, and ii) a plant lectin or a binding subunit thereof. In a specific embodiment, the mammalian sulfatase is a human sulfatase, or an enzymatically active fragment or variant thereof. In another embodiment, the enzyme is a sulfatase modifying factor. In still another embodiment the mammalian sulfatase and sulfatase modifying factor (SUMF1) are co-expressed in a plant cell so as to produce an enzymatically active sulfatase product. In one embodiment, the plant lectin is the non-toxic subunit of the lectin ricin (RTB) or nigrin (NBB). Polynucleotides encoding the fusion proteins are also contemplated for the subject invention. In one embodiment, the polynucleotide is optimized for expression in a plant, e.g., using codons preferred for plant expression.

### BRIEF DESCRIPTION OF THE DRAWINGS

FIG. 1. Western blots of crude leaf extracts (72 h post-infiltration) probed with anti-SGSH antibodies showing comparative yields of SGSH constructs (Table 6). Std, rhSGSH [100 ng]; pBK, leaves infiltrated with “empty vector” control.

FIG. 2. Western blots of crude leaf extracts (72 h post-infiltration) probed with anti-SUMF1 antibodies showing comparative yields of SUMF1 constructs (Table 7). Std, rhSUMF1 [100 ng]; pBK, leaves infiltrated with “empty vector” control.

FIG. 3. Enzyme units of plant-made SGSH. rhSGSH and rhSUMF1, mammalian cell-derived SGSH and SUMF1, respectively. 1U: sulfamidase catalyzing hydrolysis of 1 nmol of 4MU per min.

FIG. 4. Correction of MPS IIIA fibroblast cell by SGSH: RTB. Normal (Corriel #GM00010) and MPS IIIA (#GM01881) cells were incubated with SGSH constructs for 72 h. Cells were stained with Lysotracker-red and DAPI and analyzed for lysosomal volume/cell by high-through put imaging (BD Pathway 855 Bioimager). MPS IIIA cells treated with “empty vector control” fractions (pBK) was used as reference unit to estimate the impact of each treatment.

FIG. 5. Enzyme units of plant-made SGSH using viral and bacterial vectors. Timing expression of SUMF1 and SGSH using viral vector. 1U: sulfamidase catalyzing hydrolysis of 1 nmol of 4MU per min.

### BRIEF DESCRIPTION OF THE SEQUENCES

SEQ ID NO:1 is a nucleotide sequence encoding a sulfatase enzyme of the present invention.
SEQ ID NO:2 is an amino acid sequence of a sulfatase enzyme of the present invention.
SEQ ID NO:3 is a nucleotide sequence encoding a sulfatase enzyme of the present invention.
SEQ ID NO:4 is an amino acid sequence of a sulfatase enzyme of the present invention.
SEQ ID NO:5 is a nucleotide sequence encoding a sulfatase enzyme of the present invention.
SEQ ID NO:6 is an amino acid sequence of a sulfatase enzyme of the present invention.
SEQ ID NO:7 is a nucleotide sequence encoding a sulfatase enzyme of the present invention.
SEQ ID NO:8 is an amino acid sequence of a sulfatase enzyme of the present invention.
SEQ ID NO:9 is a nucleotide sequence encoding a sulfatase enzyme of the present invention.
SEQ ID NO:10 is an amino acid sequence of a sulfatase enzyme of the present invention.
SEQ ID NO:11 is a nucleotide sequence encoding a sulfatase enzyme of the present invention.
SEQ ID NO:12 is an amino acid sequence of a sulfatase enzyme of the present invention.
SEQ ID NO:13 is a nucleotide sequence encoding a sulfatase enzyme of the present invention.
SEQ ID NO:14 is an amino acid sequence of a sulfatase enzyme of the present invention.
SEQ ID NO:15 is a nucleotide sequence encoding a sulfatase enzyme of the present invention.
SEQ ID NO:16 is an amino acid sequence of a sulfatase enzyme of the present invention.
SEQ ID NO:17 is a nucleotide sequence encoding a sulfatase enzyme of the present invention.
SEQ ID NO:18 is an amino acid sequence of a sulfatase enzyme of the present invention.
SEQ ID NO:19 is a nucleotide sequence encoding a sulfatase enzyme of the present invention.
SEQ ID NO:20 is an amino acid sequence of a sulfatase enzyme of the present invention.
SEQ ID NO:21 is a nucleotide sequence encoding a sulfatase enzyme of the present invention.
SEQ ID NO:22 is an amino acid sequence of a sulfatase enzyme of the present invention.
SEQ ID NO:23 is a nucleotide sequence encoding a sulfatase enzyme of the present invention.
SEQ ID NO:24 is an amino acid sequence of a sulfatase enzyme of the present invention.
SEQ ID NO:25 is a nucleotide sequence encoding a sulfatase enzyme of the present invention.
SEQ ID NO:26 is an amino acid sequence of a sulfatase enzyme of the present invention.
SEQ ID NO:27 is a nucleotide sequence encoding a sulfatase enzyme of the present invention.
SEQ ID NO:28 is an amino acid sequence of a sulfatase enzyme of the present invention.
SEQ ID NO:29 is a nucleotide sequence encoding a sulfatase enzyme of the present invention.
SEQ ID NO:30 is an amino acid sequence of a sulfatase enzyme of the present invention.
SEQ ID NO:31 is a nucleotide sequence encoding a sulfatase enzyme of the present invention.
SEQ ID NO:32 is an amino acid sequence of a sulfatase enzyme of the present invention.
SEQ ID NO:33 is a nucleotide sequence encoding a sulfatase enzyme of the present invention.
SEQ ID NO:34 is an amino acid sequence of a sulfatase enzyme of the present invention.
SEQ ID NO:35 is a nucleotide sequence encoding a SUMF1 enzyme of the present invention.
SEQ ID NO:36 is an amino acid sequence of a SUMF1 enzyme of the present invention.
SEQ ID NO:37 is the amino acid sequence of a modified patatin sequence that can be used in the present invention.
SEQ ID NOs: 38, 40, 42, 44, 46, 48, 50, 52, 54, 56, 58, 60, 62, 64, 66, 68, 70, 72, 74, 76, 78, and 80 are nucleotide sequences of a construct of the invention as denoted in Tables 6 and 7.
SEQ ID NOs: 39, 41, 43, 45, 47, 49, 51, 53, 55, 57, 59, 61, 63, 65, 67, 69, 71, 73, 75, 77, 79, and 81 are amino acid sequences of a polypeptide encoded by a construct of the invention as denoted in Tables 6 and 7.
SEQ ID NO:82 is an ER retention sequence.

### DETAILED DESCRIPTION OF THE INVENTION

The subject invention concerns materials and methods for treating or preventing disease and conditions associated with various sulfatase enzymes that are defective or that are not properly expressed in a person or animal. In one embodiment, the disease is Sanfilippo A (MPS-IIIA) disease. The subject invention also concerns materials and methods for treating or preventing multiple sulfatase deficiency (MSD) in a person or animal. Examples of diseases and their associated enzymes are shown in Table 1.

TABLE 1

| Gene (symbol) | Accession No | Disease | Enzyme | Reference |
|---|---|---|---|---|
| Galactosamine | NM_000512 | Mucoposysaccharidosis | N-acetylgalactosamine- | [1] |
| (N-acetyl)-6 | | IVA (MPS-IVA), | 6-sulfatase | |
| sulfate sulfatase | | Morquio A syndrome | (SEQ ID NO: 2) | |
| (GALNS) | | | | |
| (SEQ ID NO: 1) | | | | |
| Glucosamine (N- | NM_002076 | Mucoposysaccharidosis | N-acetylglucosamine-6- | [2] |
| acetyl)-6 sulfatase | | IIID (MPS-IIID), | sulfatase | |
| (GNS) | | Sanfilippo D syndrome | (SEQ ID NO: 4) | |
| (SEQ ID NO: 3) | | | | |
| N-sulfoglucosamine | NM_000199 | Mucopolysaccharidosis | N-sulphoglucosamine | [3] |
| sulfohydrolase | | IIIA (MPS-IIIA), | sulphohydrolase, | |
| (SGSH) | | Sanfilippo A syndrome | sulfamidase | |
| (SEQ ID NO: 5) | | | (SEQ ID NO: 6) | |
| Sulfatase 1 | NM_015170 | NI | Extracellular sulfatase | [4] |
| (SULF1) | | | Sulf-1 (hSulf1) | |
| (SEQ ID NO: 7) | | | (SEQ ID NO: 8) | |
| Sulfatase 2 | NM_018837 | NI | Extracellular sulfatase | [4] |
| (SULF2) | | | Sulf-2 (hSulf2) | |
| (SEQ ID NO: 9) | | | (SEQ ID NO: 10) | |
| Iduronate 2- | NM_000202 | Mucopolysaccharidosis | Iduronate 2-sulfatase | [5] |
| sulfatase (IDS) | | II (MPS-II), Hunter | (SEQ ID NO: 12) | |
| (SEQ ID NO: 11) | | syndrome | | |
| Arylsulfatase A | NM_000487 | Metachromatic | Arylsulfatase A (ASA) | [6] |
| (ARSA) | | leukodystrophy (MLD) | (SEQ ID NO: 14) | |
| (SEQ ID NO: 13) | | | | |
| Arylsulfatase B | NM_000046 | Mucopolysaccharidosis | Arylsulfatase B (ASB) | [7] |
| (ARSB) | | VI (MPS-VI), | (SEQ ID NO: 16) | |
| (SEQ ID NO: 15) | | Maroteaux-Lamy | | |
| | | syndrome | | |
| Steroid sulfatase | NM_000351 | X-linked ichthyosis | Steryl-sulfatase | [8] |
| (STS) | | (XLI) | (SEQ ID NO: 18) | |
| Arylsulfatase C | | | | |
| (ARSC) | | | | |
| (SEQ ID NO: 17) | | | | |
| Arylsulfatase D | NM_001669 | NI | Arylsulfatase D (ASD) | [9] |
| (ARSD) | | | (SEQ ID NO: 20) | |
| (SEQ ID NO: 19) | | | | |
| Arylsulfatase E | NM_000047 | Chondrodysplasia | Arylsulfatase E (ASE) | [9] |
| (ARSE) | | punctata 1 (CDPX1) | (SEQ ID NO: 22) | |
| (SEQ ID NO: 21) | | | | |
| Arylsulfatase F | NM_004042 | NI | Arylsulfatase F (ASF) | [9] |
| (ARSF) | | | (SEQ ID NO: 24) | |
| (SEQ ID NO: 23) | | | | |
| Arylsulfatase G | NM_014960 | NI | Arylsulfatase G (ASG) | [10] |
| (ARSG) | | | (SEQ ID NO: 26) | |
| (SEQ ID NO: 25) | | | | |
| Arylsulfatase H | NM_001011719 | NI | Arylsulfatase H (ASH) | [11] |
| (ARSH) | | | (SEQ ID NO: 28) | |
| (SEQ ID NO: 27) | | | | |
| Arylsulfatase I | NM_001012301 | NI | Arylsulfatase I (ASI) | [11] |
| (ARSI) | | | (SEQ ID NO: 30) | |
| (SEQ ID NO: 29) | | | | |
| Arylsulfatase J | NM_024590 | NI | Arylsulfatase J (ASJ) | [11] |
| (ARSJ) | | | (SEQ ID NO: 32) | |
| (SEQ ID NO: 31) | | | | |
| Arylsulfatase K | NM_198150 | NI | Arylsulfatase K (ASK) | [11] |
| (ARSK) | | | (SEQ ID NO: 34) | |
| (SEQ ID NO: 33) | | | | |
| Sulfatase | NM_182760 | Multiple sulfatase | Sulfatase-modifying | [12, 13] |
| modifying factor | | deficiency (MSD) | factor 1 | |
| 1 (SUMF1) | | | C-α-formylglycine- | |
| (SEQ ID NO: 35) | | | generating enzyme | |
| | | | (FGE) (SEQ ID NO: 36) | |

NI, not identified

Compounds within the scope of the invention include, but are not limited to a mammalian sulfatase and/or sulfatase modifying factor 1 (SUMF1) or an enzymatically active fragment or variant thereof, or a fusion protein comprising i) a mammalian sulfatase protein, or an enzymatically active fragment or variant thereof, and ii) a plant lectin or a binding subunit thereof. In one embodiment, the sulfatase, or fusion protein containing the sulfatase, are co-expressed with the SUMF1 so as to activate the sulfatase during synthesis. In another embodiment, a fusion protein comprises i) a mammalian sulfatase modifying factor 1 (SUMF1) protein, or an enzymatically active fragment or variant thereof, and ii) a plant lectin or a binding subunit thereof. The mammalian sulfatase can be one that normalizes the cellular phenotype of a lysosomal disease when expressed in a cell or that reduces the symptoms of a lysosomal disease in an animal or human (examples of diseases are shown in Table 1). In one embodiment, the sulfatase is activated by a co-expressed SUMF1 enzyme by converting a cysteine at the active site to a formyl glycine amino acid. Examples of sulfatases contemplated by the present invention are shown in Table 1. Optionally, the sulfatase or the SUMF1 protein can be linked to the plant lectin by a linker sequence of amino acids. In a specific embodiment, the mammalian protein is a human protein, or an enzymatically active fragment or variant thereof. In one embodiment, the mammalian SUMF1 protein or the SUMF1 fusion protein comprises an ER retention sequence, such as KDEL (SEQ ID NO:82). In a specific embodiment, the ER retention sequence is located at the C-terminus of the SUMF1 or SUMF1 fusion protein. In some embodiments, the mammalian sulfatase comprises the amino acid sequence shown in any of SEQ ID NOs: 2, 4, 6, 8, 10, 12, 14, 16, 18, 20, 22, 24, 26, 28, 30, 32, or 34, or an enzymatically active fragment or variant thereof. In some embodiments, the SUMF1 protein comprises the amino acid sequence shown in SEQ ID NO:36, or an enzymatically active fragment or variant thereof. The plant lectin portion of the fusion protein can be any plant lectin such as those described herein. In one embodiment, the plant lectin is the non-toxic B subunit of the lectin ricin (RTB) or nigrin (NBB). Amino acid sequences of numerous plant lectins, and nucleotide sequences encoding them, are known in the art. In specific embodiments, the fusion protein comprises the amino acid sequence shown in any of SEQ ID NOs: 39, 41, 43, 45, 47, 49, 51, 53, 55, 57, 59, 61, 63, 65, 67, 69, 71, 73, 75, 77, 79, or 81, or an enzymatically active fragment or variant thereof. Polynucleotides encoding the fusion proteins are also contemplated for the subject invention. In some embodiments, the polynucleotides comprise the protein encoding nucleotide sequence of any of SEQ ID NOs: 1, 3, 5, 7, 9, 11, 13, 15, 17, 19, 21, 23, 25, 27, 29, 31, 33, or 35. In one embodiment, the polynucleotide is optimized for expression in a plant, e.g., using codons preferred for plant expression. In a specific embodiment, the polynucleotide is optimized for expression in Nicotiana Sp. In a more specific embodiment, the polynucleotide is optimized for expression in Nicotiana benthamiana. In one embodiment, a polynucleotide of the invention comprises the nucleotide sequence of any of SEQ ID NOs: 38, 40, 42, 44, 46, 48, 50, 52, 54, 56, 58, 60, 62, 64, 66, 68, 70, 72, 74, 76, 78, or 80. In one embodiment, the fusion protein is produced in plants using a plant-based expression system such as described in U.S. Pat. No. 5,929,304.

In one embodiment, a compound of the invention comprises a sulfatase or a fusion protein wherein the sulfatase is heparan N-sulfatase, or the fusion protein comprises i) the enzyme heparan N-sulfatase (SGSH), or an enzymatically active fragment or variant thereof, and ii) a plant lectin or a binding subunit thereof. In a more specific embodiment, the heparan N-sulfatase comprises the amino acid sequence shown in SEQ ID NO:6, or an enzymatically active fragment or variant thereof. In a specific embodiment, the heparan N-sulfatase is a human heparan N-sulfatase, or an enzymatically active fragment or variant thereof. In one embodiment, the plant lectin is the non-toxic B subunit lectin of ricin (RTB) or nigrin (NBB). In one embodiment, the SGSH portion and the plant lectin portion of the fusion protein can be linked by a linker sequence of amino acids. In one embodiment of the invention, a fusion protein with SUMF1 comprises an ER retention sequence, such as KDEL (SEQ ID NO:82). In a specific embodiment, the ER retention sequence is located at the C-terminus of the fusion protein.

The subject invention also concerns a mammalian sulfatase modifying factor 1 (SUMF1), or an enzymatically active fragment or variant thereof. In one embodiment, the mammalian SUMF1 protein is a human SUMF1 protein. In a specific embodiment, a SUMF1 protein comprises the amino acid sequence shown in SEQ ID NO:36. The subject invention also concerns polynucleotides encoding a SUMF1 protein. In one embodiment, a polynucleotide of the invention comprises the nucleotide sequence shown in SEQ ID NO:35. In one embodiment, the polynucleotide is optimized for expression in a plant, e.g., using codons preferred for plant expression. In one embodiment, the polynucleotide is optimized for expression in Nicotiana sp. In a specific embodiment, the polynucleotide is optimized for expression in N. benthamiana (SEQ ID NO:40). In one embodiment, a SUMF1 protein of the invention comprises an ER retention sequence, such as KDEL (SEQ ID NO:82). In a specific embodiment, the ER retention sequence is located at the C-terminus of the SUMF1 protein (SEQ ID NOs: 79 and 81).

The subject invention also concerns methods for treating or preventing diseases or conditions associated with sulfatase enzymes, such as MPS-IIIA disease, in a person or animal (e.g., a disease where the sulfatase enzyme is defective or non-functional or partially functional). Examples of diseases and the associated enzymes are shown in Table 1. In one embodiment, the method comprises administering a therapeutically effective amount of a sulfatase or a fusion protein of the present invention, or an enzymatically active fragment or variant thereof, to the person or animal. In one embodiment, the sulfatase or fusion protein comprises a human sulfatase. Human sulfatases that can be used in the subject method include, but are not limited to, those shown in Table 1. Human sulfatases contemplated for use in the fusion protein include, but are not limited to, those shown in Table 1. In specific embodiments, the sulfatase or fusion protein comprises a sulfatase sequence shown in any of SEQ ID NOs: 2, 4, 6, 8, 10, 12, 14, 16, 18, 20, 22, 24, 26, 28, 30, 32, or 34, or an enzymatically active fragment or variant thereof. In one embodiment, the sulfatase or fusion protein is administered intravenously, by injection or infusion, or by inhalation via nasal cavity or lung, or orally, ocularly, vaginally, anally, rectally, or transmembraneously or transdermally, subcutaneously, intradermally, intravenously, intramuscularly, intraperitoneally, or intrasternally, such as by injection. In one embodiment, the person is a fetus, a newborn, or an infant. Optionally, the methods include screening the person or animal to determine if it has a disease or condition associated with sulfatase enzymes. In one embodiment, the method reduces disease phenotype in cells and tissue of the body. In a further embodiment, the method reduces disease symptoms in the central nervous system and/or brain.

The subject invention also concerns methods for treating multiple sulfatase deficiency (MSD) in a person or animal. In one embodiment, the method comprises administering a therapeutically effective amount of a mammalian SUMF1 protein, or an enzymatically active fragment or variant thereof, to the person or animal. In another embodiment, the method comprises administering a therapeutically effective amount of a fusion protein to the person or animal, wherein the fusion protein comprises i) a mammalian SUMF1 protein, or an enzymatically active fragment or variant thereof, and ii) a plant lectin or a binding subunit thereof. In one embodiment, the mammalian SUMF1 is a human SUMF1. Optionally, the SUMF1 protein or SUMF1 fusion protein can comprise an ER retention sequence, such as KDEL (SEQ ID NO:82). In one embodiment, the ER retention sequence is located at the C-terminus of the protein. In one embodiment, the SUMF1 protein or SUMF1 fusion protein is expressed in a plant cell. In another embodiment, a SUMF1 protein or SUMF1 fusion protein is expressed in an animal cell. In a specific embodiment, the human SUMF1 protein or SUMF1 fusion protein comprises the amino acid sequence in SEQ ID NO:36, or an enzymatically active fragment or variant thereof. In one embodiment, the SUMF1 protein or the fusion protein is administered to the person or animal via intravenous injection or infusion. In one embodiment, the person is a fetus, a newborn, or an infant. Optionally, the methods include screening the person or animal to determine if it has MSD disease or a condition associated with MSD.

The subject invention also concerns methods for preparing sulfatase enzymes and sulfatase fusion proteins of the present invention. In one embodiment, a method comprises transforming a plant or plant cell with i) polynucleotide encoding a sulfatase enzyme or a sulfatase fusion protein of the invention, and ii) a polynucleotide encoding a mammalian sulfatase modifying factor 1 (SUMF1) or a SUMF1 fusion protein of the invention; and expressing the sulfatase or sulfatase fusion protein and the SUMF1 protein or SUMF1 fusion protein in the plant. Methods for transforming a plant or plant cell with a polynucleotide are known in the art and include, for example, Agrobacterium infection, biolistic methods, and electroporation. The plant or plant cell can be transiently or stably transformed with the polynucleotide(s). In another embodiment, a method comprises using a plant or plant cell that has a polynucleotide encoding a mammalian SUMF1 protein or a SUMF1 fusion protein stably incorporated into its genome and that expresses SUMF1, and transforming the plant or plant cell with a polynucleotide encoding a sulfatase enzyme or a sulfatase fusion protein of the invention, and expressing the sulfatase or sulfatase fusion protein in the plant or plant cell, wherein the sulfatase or sulfatase fusion protein is activated by the expressed SUMF1 or SUMF1 fusion protein. In a further embodiment, a method comprises using a plant or plant cell that has a polynucleotide encoding a mammalian sulfatase enzyme or a sulfatase fusion protein of the invention stably incorporated into its genome and that expresses the sulfatase enzyme or the sulfatase fusion protein, and transforming the plant or plant cell with a polynucleotide encoding a mammalian SUMF1 protein or a SUMF1 fusion protein of the invention, and expressing the SUMF1 or SUMF1 fusion protein in the plant or plant cell, wherein the sulfatase or sulfatase fusion protein is activated by the expressed SUMF1 or SUMF1 fusion protein. In a further embodiment, a method comprises using a plant or plant cell that has i) a polynucleotide encoding a mammalian SUMF1 protein or a SUMF1 fusion protein stably incorporated into its genome and that expresses SUMF1 and that has ii) a polynucleotide encoding a mammalian sulfatase enzyme or a sulfatase fusion protein of the invention stably incorporated into its genome and that expresses the sulfatase enzyme or the sulfatase fusion protein, wherein the expressed sulfatase or sulfatase fusion protein is activated by the expressed SUMF1 or SUMF1 fusion protein. Methods for stably incorporating a polynucleotide into the genome of a plant or plant cell are known in the art. The polynucleotides utilized in the methods can be provided in an expression construct. In one embodiment, the cells are grown in tissue culture. In another embodiment, the cells are grown in a bioreactor.

Following transient or stable expression in the plant or plant cell, the sulfatase enzyme or sulfatase fusion protein and/or the SUMF1 protein or the SUMF1 fusion protein can be isolated from the plant. In one embodiment, transient expression of the enzyme or fusion protein in the plant or plant cell occurs for 1 to 5 days (typically, 2 to 5 days) prior to isolation of the enzyme or fusion protein from the plant or plant cell. Methods for protein isolation and purification are known in the art and include, for example, affinity chromatography. Co-expression of the sulfatase or sulfatase fusion protein and SUMF1 or SUMF1 fusion protein results in activation of the sulfatase or sulfatase fusion protein by the SUMF1 or SUMF1 fusion protein. The activated sulfatase or sulfatase fusion protein can be used to treat or prevent diseases or conditions in a person or animal that are associated with defective sulfatases and/or improper expression of sulfatases. Plants and plant cells that can be used in the synthesis methods include, but are not limited to, rice, wheat, barley, oats, rye, sorghum, maize, sugarcane, pineapple, onion, bananas, coconut, lilies, turfgrasses, millet, tomato, cucumber, squash, peas, alfalfa, melon, chickpea, chicory, clover, kale, lentil, soybean, beans, tobacco, potato, sweet potato, yams, cassava, radish, broccoli, spinach, cabbage, rape, apple trees, citrus (including oranges, mandarins, grapefruit, lemons, limes and the like), grape, cotton, sunflower, strawberry, lettuce, and hop. In one embodiment, the plant is a Nicotiana sp. In a specific embodiment, the plant is N. benthamiana.

The subject invention also concerns methods for producing a SUMF1 protein or a SUMF1 fusion protein of the present invention. In one embodiment, a method comprises transforming a cell with a polynucleotide encoding a SUMF1 protein or a SUMF1 fusion protein, or an enzymatically active fragment or variant thereof, and expressing the SUMF1 protein or the SUMF1 fusion protein in the cell. Following expression, the SUMF1 or SUMF1 fusion protein can be isolated from the cell. Optionally, the SUMF1 or SUMF1 fusion protein can be co-expressed in the cell along with a sulfatase enzyme or sulfatase fusion protein of the present invention. In one embodiment, the cell is a plant cell. The cell can be transiently or stably transformed with the polynucleotide. In one embodiment, the cell is an animal cell. In a specific embodiment, the animal cell is a cell line, such as a mammalian cell line (e.g., Chinese hamster ovary (CHO) cell line). In one embodiment, the cells are grown in tissue culture. In another embodiment, the cells are grown in a bioreactor. In one embodiment, the mammalian SUMF1 is a human SUMF1. Optionally, the SUMF1 protein can comprise an ER retention sequence, such as KDEL (SEQ ID NO:82) located at the C-terminus of the protein. Plants and plant cells that can be used in the synthesis methods include, but are not limited to, rice, wheat, barley, oats, rye, sorghum, maize, sugarcane, pineapple, onion, bananas, coconut, lilies, turfgrasses, millet, tomato, cucumber, squash, peas, alfalfa, melon, chickpea, chicory, clover, kale, lentil, soybean, beans, tobacco, potato, sweet potato, yams, cassava, radish, broccoli, spinach, cabbage, rape, apple trees, citrus (including oranges, mandarins, grapefruit, lemons, limes and the like), grape, cotton, sunflower, strawberry, lettuce, and hop. In one embodiment, the plant is a Nicotiana sp. In a specific embodiment, the plant is N. benthamiana.

Plant lectins for use in the fusion proteins that are contemplated within the scope of the invention include, but are not limited to, those B subunits from AB toxins such as ricins, abrins, nigrins, and mistletoe toxins, viscumin toxins, ebulins, pharatoxin, hurin, phasin, and pulchellin. They may also include lectins such as wheat germ agglutinin, peanut agglutinin, and tomato lectin that, while not part of the AB toxin class, are still capable of binding to animal cell surfaces and mediating endocytosis and transcytosis. Specific examples of plant lectins including their binding affinities and trafficking behavior are discussed further below. Therapeutic compounds and agents contemplated within the scope of the invention include, but are not limited to large molecular weight molecules including therapeutic proteins and peptides. Examples of therapeutic compounds and agents are discussed further below.

Within the scope of the present invention, selection of a specific plant lectin candidate to use in delivery of a particular therapeutic compound or agent is based on the specific sugar affinity of the lectin, its uptake efficiency into specific target cells, its pattern of intracellular trafficking, its in vivo biodistribution and pharmacodynamics, or other features of the lectin or therapeutic compound. Alternatively, multiple lectins can be tested to identify the lectin—therapeutic compound combination that provides greatest efficacy. For example, two lectins, RTB and NNB, were selected for proof-of-concept of the invention based on trafficking of their respective AB toxins, ricin from Ricinus communis and nigrin-b from Sambucus nigra (e.g., see Sandvig, K. and van Deurs, B. (1999); Simmons et al. (1986); Citores et al. (1999); Citores et al. (2003)). The uptake and trafficking of ricin and/or RTB, a galactose/galactosamine-specific lectin, has been extensively studied. This lectin has high affinity for surface glycolipids and glycoproteins providing access to a broad array of cells and enters cells by multiple endocytotic routes. These include absorptive-mediated endocytosis involving clathrin-dependent and clathrin-independent routes; caveolin-dependent and independent routes; dynamin-dependent and independent routes, and macropinocytosis based on the lectin binding to cell surface glycoproteins and glycolipids. RTB also accesses cells by receptor-mediated endocytosis based on interaction with its N-linked glycans with the high-mannose receptor (MMR) of animal cells. Upon endocytosis, RTB traverses preferentially to lysosomes (lysosomal pathway) or cycles back to the cell membrane (transcytosis pathway), with a small amount (generally less than 5%) moving “retrograde” to the endoplasmic reticulum. The NBB lectin, Nigrin B B-subunit from Sambucus nigra, exploits different uptake and intracellular trafficking routes compared to RTB, and thus provides unique in vivo pharmacodynamics. In contrast to RTB, NBB has strong affinity for N-acetyl-galactosamine, low affinity for lactose, very limited retrograde trafficking but strong accumulation in lysosomes. Plant-made DsRED:NNB (red fluorescent protein—NBB fusion) is rapidly taken up into multiple mammalian cells and efficiently delivered to lysosomes. Recombinantly produced RTB and NBB have been operatively associated with both small molecules (by chemical conjugation technologies) and protein macromolecule by genetic fusion that retain selective lectin binding as well as functionality of the associated protein or agent. These operatively associated products are rapidly endocytosed into multiple cell types and tissues and deliver fully functional ‘payload’ into internal structures including lysosomes, endosomes, endoplasmic reticulum, and sarcoplasmic reticulum. Of particular significance, these lectins mobilize delivery of enzymes and other large proteins into “hard-to-treat” cells of the central nervous system (including, but not limited to, brain capillary endothelial cells, neurons, glial cells, and astrocytes), skeletal systems (including, but not limited to, cartilage, osteoblasts, chondrocytes, fibroblasts, and monocytes), and the respiratory system (including, but not limited to, lung airway epithelium, lung smooth muscle cells, and macrophages) (Radin et al., unpublished). These cells and tissues represent some of the most challenging targets for delivery of therapeutic agents highlighting the utility and novelty of the invention to address currently unmet needs in therapeutic compound delivery in human and animal medicine.

Additional plant lectins that are contemplated within the scope of the invention are those having particular carbohydrate binding affinities including, but not limited to, lectins that bind glucose, glucosamine, galactose, galactosamine, N-acetyl-glucosamine, N-acetyl-galactosamine, mannose, fucose, sialic acid, neuraminic acid, and/or N-acetylneuraminic acid, or have high affinity for certain target tissue or cells of interest. There are hundreds of plant lectins that have been identified and experimental strategies to identify plant lectins, their respective genes, and their sugar binding affinities are widely known by those skilled in the art. The diversity of plant sources for lectins and their sugar binding affinities is exemplified in Table 2 (adapted from Table 3 of Van Damme et al., (1998)).

TABLE 2
Type 2 Ribosome-Inactivating Proteins and Related Lectins:
Occurrence, Molecular Structure, and Specificity

| | | | | Sequence |
|---|---|---|---|---|
| Species | Tissue | Structure^{a} | Specificity | available^{b} |
| Merolectins | | | | |
| Sambucus nigra | Bark | [P22] | NANA | Nu |
| | Fruit | [P22] | NANA | Nu |
| Hololectins | | | | |
| Sambucus nigra | Bark | II [P30]₂ | GaINAc > GaI | Nu |
| | Seed | III [P30]₂ | GaINAc > GaI | |
| | Fruit | IVf [P32]₂ | GaI/GaINAc | Nu (SNA-IV) |
| | Leaf | IVI [P32]₂ | GaI/GaINAc | Nu |
| | Leaf | IV4I [P32]₄ | GaI/GaINAc | |
| Chimerolectins | | | | |
| Abrus precatorius | Seed | [P(34 + 32)] | GaI > GaINAc | Pr, Nu (Abrin) |
| | Seed | [P(33 + 29)]₂ | GaI | Pr (APA) |
| Adenia digitata | Root | [P(28 + 38)] | GaI > GaINAc | |
| Adenia volkensii | Root | [P(28 + 38)] | GaI | |
| Cinnamomum camphora | Seed | [P(30 + 33)]_{?} | Unknown | |
| Eranthis hyemalis | Tuber | [P(30 + 32)] | GaINAc | |
| Iris hybrid | Bulb | [P(27 + 34)] | GaINAc | |
| Momordica charantia | Seed | [P(28 + 30)]₂ | GaI > GaINAc | |
| Phoradendron californicum | Plant | [P(31 + 38)] | GaI | |
| Ricinus communis | Seed | [P(32 + 34)] | GaI > GaINAc | Pr, Nu (Ricin) |
| | Seed | [P(32 + 38)]₂ | GaI >> GaINAc | Pr, Nu (RCA) |
| Sambucus canadensis | Bark | I [P(32 + 35)]₂ | NANA | |
| Sambucus ebulus | Bark | I [P(32 + 37)]₂ | NANA | |
| | Leaf | [P(26 + 30)]₂ | GaINAc | |
| Sambucus nigra | Seed | Va [P(26 + 32)]₂ | GaINAc > GaI | |
| | Bark | I [P(32 + 35)]_{c} | NANA | Nu (SNA-I) |
| | Bark | I′ [P(32 + 35)]₂ | NANA | Nu (SNA-I′) |
| | Bark | V [P(26 + 32)]₃ | GaINAc > GaI | Nu (SNA-V) |
| | Fruit | If [P(32 + 35)]₂ | NANA | Nu |
| | Fruit | Vf [P(26 + 32)]₂ | GaINAc > GaI | Nu |
| Sambucus racemosa | Bark | I [P(30 + 38)]₄ | NANA | |
| Sambucus sieboldiana | Bark | I [P(31 + 37)]₄ | NANA | Nu (SSA-I) |
| | Bark | [P(27 + 32)] | GaINAc > GaI | Nu (Sieboldin) |
| Viscum album | Plant | I [P(29 + 34)]₁₋₂ | GaI | |
| | Plant | II [P(29 + 34)] | GaI/GaINAc | |
| | Plant | III [P(25 + 30)] | GaINAc > GaI | |
| Type 2 RIP with inactive B chain | | | | |
| Sambucus nigra | Bark | [P(32 + 32)] | — | Nu (LRPSN) |

^{a}[PX] stands for promoter with a molecular mass of X kDa. [P(Y + Z)] indicates that the promoter is cleaved in two polypeptides of Y and Z kDa.
^{b}Pr. proton sequence; Nu, nucleotoids sequence. The abbreviation in brackets refers to the sequence name used in the dendrogram (FIG. 20).

As a further example of plant lectins contemplated herein. Table 3 exemplifies the large number of different lectins identified from the Sambucus species alone. This group includes nigrin B. the source on NBB.

TABLE 3
Ribosome-inactivating proteins (RIPs) and lectins from Sambucus
species. Adapted from Table 1 of Ferreras et al. (2011)

| Proteins | Species | Tissues |
|---|---|---|
| Type 1 RIPs | | |
| Ebulitins α, β and γ | S. ebulus | Leaves |
| Nigritius f1 and f2 | S. nigra | Fruits |
| Heterodimeric type 2 RIPs | | |
| Ebulin l | S. ebulus | Leaves |
| Ebulin f | S. ebulus | Fruits |
| Ebulins r1 and r2 | S. ebulus | Rhizome |
| Nigrin b, basic nigrib b, SNA I′, SNLRPs | S. nigra | Bark |
| Nigrins I1 and I2 | S. nigra | Leaves |
| Nigrin f | S. nigra | Fruits |
| Nigrin s | S. nigra | Seeds |
| Sieboldin b | S. sieboldiana | Bark |
| Basic racemosin b | S. racemosa | Bark |
| Tetrameric type 2 RIPs | | |
| SEA | S. ebulus | Rhizome |
| SNA I | S. nigra | Bark |
| SNAIf | S. nigra | Fruits |
| SNAflu-I | S. nigra | Flowers |
| SSA | S. sieboldiana | Bark |
| SRA | S. racemosa | Bark |
| Monomeric lectins | | |
| SELIm | S. ebulus | Leaves |
| SEA II | S. ebulus | Rhizome |
| SNA II | S. nigra | Bark |
| SNAIm and SNAIVI | S. nigra | Leaves |
| SNA IV | S. nigra | Fruits |
| SNA III | S. nigra | Seeds |
| SSA-b-3 and SSA-b-4 | S. sieboldiana | Bark |
| SRAbm | S. racemosa | Bark |
| Homodimeric lectins | | |
| SELId | S. ebulus | Leaves |
| SELfd | S. ebulus | Fruits |
| SNAId | S. nigra | Leaves |

The subject invention also concerns polynucleotides that comprise nucleotide sequences encoding a sulfatase and/or a SUMF1 protein and/or fusion protein (or compound) of the invention. In one embodiment, the polynucleotides comprise nucleotide sequences that are optimized for expression in a particular expression system, e.g., a plant expression system, such as a tobacco plant. In one embodiment, the polynucleotide is optimized for expression in Nicotiana sp. In a specific embodiment, the polynucleotide is optimized for expression in N. benthamiana. The subject invention also concerns the sulfatases, SUMF1 proteins, and fusion polypeptides encoded by polynucleotides of the invention.

The present invention contemplates products in which the plant lectin is operatively associated with the therapeutic component by one of many methods known in the art. For example, genetic fusions between a plant lectin protein and a therapeutic protein can orient the lectin partner on either the C- or N-terminus of the therapeutic component. The coding regions can be linked precisely such that the last C-terminal residue of one protein is adjacent to the first N-terminal residue of the mature (i.e., without signal peptide sequences) second protein. Alternatively, additional amino acid residues can be inserted between the two proteins as a consequence of restriction enzyme sites used to facilitate cloning at the DNA level. Additionally, the fusions can be constructed to have amino acid linkers between the proteins to alter the physical spacing. These linkers can be short or long, flexible (e.g., the commonly used (Gly₄Ser)₃ ‘flexi’ linker) or rigid (e.g., containing spaced prolines), provide a cleavage domain (e.g., see Chen et al. (2010)), or provide cysteines to support disulfide bond formation. The plant lectins are glycoproteins and in nature are directed through the plant endomembrane system during protein synthesis and post-translational processing. For this reason, production of recombinant fusion proteins comprising a plant lectin and a therapeutic protein partner may require that a signal peptide be present on the N-terminus of the fusion product (either on the lectin or on the therapeutic protein depending on the orientation of the fusion construct) in order to direct the protein into the endoplasmic reticulum during synthesis. This signal peptide can be of plant or animal origin and is typically cleaved from the mature plant lectin or fusion protein product during synthesis and processing in the plant or other eukaryotic cell. In one embodiment, a modified patatin signal sequence (PoSP) is utilized: MASSATTKSFLILFFMILATTSSTCAVD (SEQ ID NO:37) (see GenBank accession number CAA27588.1, version GI: 21514 by Bevan et al. and referenced at “The structure and transcription start site of a major potato tuber protein gene” Nucleic Acid Res. 14 (11), 4625-4638 (1986)).

Compounds of the subject invention can also be prepared by producing the plant lectin and the therapeutic drug or protein separately and operatively linking them by a variety of chemical methods. Examples of such in vitro operative associations include conjugation, covalent binding, protein-protein interactions or the like (see, e.g., Lungwitz et al. (2005); Lovrinovic and Niemeyer (2005)). For example, N-hydroxysuccinimde (NHS)-derivatized small molecules and proteins can be attached to recombinant plant lectins by covalent interactions with primary amines (N-terminus and lysine residues). This chemistry can also be used with NHS-biotin to attach biotin molecules to the plant lectin supporting subsequent association with streptavidin (which binds strongly to biotin) and which itself can be modified to carry additional payload(s). In another example, hydrazine-derivatized small molecules or proteins can be covalently bound to oxidized glycans present on the N-linked glycans of the plant lectin. Proteins can also be operatively linked by bonding through intermolecular disulfide bond formation between a cysteine residue on the plant lectins and a cysteine residue on the selected therapeutic protein. It should be noted that the plant AB toxins typically have a single disulfide bond that forms between the A and B subunits. Recombinant production of plant B subunit lectins such as RTB and NBB yield a product with an ‘unpaired’ cysteine residue that is available for disulfide bonding with a “payload” protein. Alternatively, this cysteine (e.g., Cys₄ in RTB) can be eliminated in the recombinant plant lectin product by replacement with a different amino acid or elimination of the first 4-6 amino acids of the N-terminus to eliminate the potential for disulfide bonding with itself or other proteins.
NBB: See GenBank accession number P33183.2, version GI: 17433713 (containing subunits A and B) by Van Damme et al. and referenced at “Characterization and molecular cloning of Sambucus nigra agglutinin V (nigrin b), a GalNAc-specific type-2 ribosome-inactivating protein from the bark of elderberry (Sambucus nigra)” Eur. J. Biochem. 237 (2), 505-513 (1996). PDB ID: 3CA3 (for B subunit) by Maveyraud et al. and referenced at “Structural basis for sugar recognition, including the tn carcinoma antigen, by the lectin sna-ii from sambucus nigra” Proteins 75 p.89 (2009).
SGSH: See GenBank accession number NP_000190.1, version GI: 4506919 by Van de Kamp et al. and referenced at “Genetic heterogeneity and clinical variability in the Sanfilippo syndrome (type A, B, and C)” Clin. Genet. 20 (2), 152-160 (1981).
RTB: See GenBank accession number pbd/2AAI/B, version GI: 494727 (containing subunits A and B) by Montfort et al. and referenced at “The three-dimensional structure of ricin at 2.8A” J. Biol Chem. 262 (11), 5398-5403 (1987).

In vivo administration of the subject compounds, polynucleotides and compositions containing them, can be accomplished by any suitable method and technique presently or prospectively known to those skilled in the art. The subject compounds can be formulated in a physiologically- or pharmaceutically-acceptable form and administered by any suitable route known in the art including, for example, oral, nasal, rectal, transdermal, vaginal, and parenteral routes of administration. As used herein, the term parenteral includes subcutaneous, intradermal, intravenous, intramuscular, intraperitoneal, and intrasternal administration, such as by injection. Administration of the subject compounds of the invention can be a single administration, or at continuous or distinct intervals as can be readily determined by a person skilled in the art. In one embodiment, a polynucleotide encoding a therapeutic fusion product of the invention is stably incorporated into the genome of a person of animal in need of treatment. Methods for providing gene therapy are well known in the art. In one embodiment, a polynucleotide is provided in an expression construct and encodes an amino acid sequence of any of SEQ ID NOs: 2, 4, 6, 8, 10, 12, 14, 16, 18, 20, 22, 24, 26, 28, 30, 32, 34, or 36, or an enzymatically active fragment or variant thereof.

The compounds of the subject invention, and compositions comprising them, can also be administered utilizing liposome and nano-technology, slow release capsules, implantable pumps, and biodegradable containers, and orally or intestinally administered intact plant cells expressing the therapeutic product. These delivery methods can, advantageously, provide a uniform dosage over an extended period of time.

Compounds of the subject invention can be formulated according to known methods for preparing physiologically acceptable compositions. Formulations are described in detail in a number of sources which are well known and readily available to those skilled in the art. For example, Remington's Pharmaceutical Science by E.W. Martin describes formulations which can be used in connection with the subject invention. In general, the compositions of the subject invention will be formulated such that an effective amount of the compound is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional physiologically-acceptable carriers and diluents which are known to those skilled in the art. Examples of carriers or diluents for use with the subject compounds include ethanol, dimethyl sulfoxide, glycerol, alumina, starch, saline, and equivalent carriers and diluents. To provide for the administration of such dosages for the desired therapeutic treatment, compositions of the invention will advantageously comprise between about 0.1% and 99%, and especially, 1 and 15% by weight of the total of one or more of the subject compounds based on the weight of the total composition including carrier or diluent.

Compounds and agents of the invention, and compositions thereof, may be locally administered at one or more anatomical sites, optionally in combination with a pharmaceutically acceptable carrier such as an inert diluent. Compounds and agents of the invention, and compositions thereof, may be systemically administered, such as intravenously or orally, optionally in combination with a pharmaceutically acceptable carrier such as an inert diluent, or an assimilable edible carrier for oral delivery. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, aerosol sprays, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

Compounds and agents, and compositions of the invention, including pharmaceutically acceptable salts or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent of the invention in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds and agents and pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The present invention also concerns pharmaceutical compositions comprising a compound and/or agent of the invention in combination with a pharmaceutically acceptable carrier. Pharmaceutical compositions adapted for oral, topical or parenteral administration, comprising an amount of a compound constitute a preferred embodiment of the invention. The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

To provide for the administration of such dosages for the desired therapeutic treatment, in some embodiments, pharmaceutical compositions of the invention can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and acrosol, 0.01 to about 20 mg/kg of animal (body) weight.

The subject invention also concerns kits comprising a compound and/or composition and/or agent and/or polynucleotide of the invention in one or more containers. Kits of the invention can optionally include pharmaceutically acceptable carriers and/or diluents. In one embodiment, a kit of the invention includes one or more other components, adjuncts, or adjuvants as described herein. In one embodiment, a kit of the invention includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent and/or polynucleotide of the invention is provided in the kit as a solid, such as a tablet, pill, or powder form. In another embodiment, a compound and/or agent and/or polynucleotide of the invention is provided in the kit as a liquid or solution. In one embodiment, the kit comprises an ampoule or syringe containing a compound and/or agent of the invention in liquid or solution form.

Mammalian species which benefit from the disclosed methods include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Other species that may benefit from the disclosed methods include fish, amphibians, avians, and reptiles. As used herein, the terms “patient” and “subject” are used interchangeably and are intended to include such human and non-human species. Likewise, in vitro methods of the present invention can be carried out on cultured cells or tissues of such human and non-human species.

The subject invention also concerns bacterial cells, and animals, animal tissue, and animal cells, and plants, plant tissue, and plant cells of the invention that comprise or express a polynucleotide or the protein encoded by the polynucleotide of the invention, or a fragment or variant thereof. Plant tissue includes, but is not limited to, leaf, stem, seed, scion, roots, and rootstock. Plants within the scope of the present invention include monocotyledonous plants, such as, for example, rice, wheat, barley, oats, rye, sorghum, maize, sugarcane, pineapple, onion, bananas, coconut, lilies, turfgrasses, and millet. Plants within the scope of the present invention also include dicotyledonous plants, such as, for example, tomato, cucumber, squash, peas, alfalfa, melon, chickpea, chicory, clover, kale, lentil, soybean, beans, tobacco, potato, sweet potato, yams, cassava, radish, broccoli, spinach, cabbage, rape, apple trees, citrus (including oranges, mandarins, grapefruit, lemons, limes and the like), grape, cotton, sunflower, strawberry, lettuce, and hop. In one embodiment, the plant is a Nicotiana sp. In a specific embodiment, the plant is N. benthamiana. Herb plants containing a polynucleotide of the invention are also contemplated within the scope of the invention. Herb plants include parsley, sage, rosemary, thyme, and the like. Trees are also contemplated within the scope of the subject invention. In one embodiment, a plant, plant tissue, or plant cell is a transgenic plant, plant tissue, or plant cell. In another embodiment, a plant, plant tissue, or plant cell is one that has been obtained through a breeding program.

Polynucleotides encoding a sulfatase, a SUMF1 protein, and/or a fusion product of the present invention, or an enzymatically active fragment or variant thereof, can be provided in an expression construct. Expression constructs of the invention generally include regulatory elements that are functional in the intended host cell in which the expression construct is to be expressed. Thus, a person of ordinary skill in the art can select regulatory elements for use in bacterial host cells, yeast host cells, plant host cells, insect host cells, mammalian host cells, and human host cells. Regulatory elements include promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation. In one embodiment, an expression construct comprises a polynucleotide encoding an amino acid sequence of any of SEQ ID NOs: 2, 4, 6, 8, 10, 12, 14, 16, 18, 20, 22, 24, 26, 28, 30, 32, 34, or 36, or an enzymatically active fragment or variant thereof.

An expression construct of the invention can comprise a promoter sequence operably linked to a polynucleotide sequence of the invention, for example a sequence encoding a fusion polypeptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct.

Constitutive promoters (such as the CaMV, ubiquitin, actin, or NOS promoter), developmentally-regulated promoters, and inducible promoters (such as those promoters that can be induced by heat, light, hormones, or chemicals) are also contemplated for use with polynucleotide expression constructs of the invention. If the expression construct is to be provided in or introduced into a plant cell, then plant viral promoters, such as, for example, a cauliflower mosaic virus (CaMV) 35S (including the enhanced CaMV 35S promoter (see, for example U.S. Pat. No. 5,106,739)) or a CaMV 19S promoter or a cassava vein mosaic can be used. Other promoters that can be used for expression constructs in plants include, for example, prolifera promoter, Ap3 promoter, heat shock promoters, T-DNA 1′- or 2′-promoter of A. tumefaciens, polygalacturonase promoter, chalcone synthase A (CHS-A) promoter from petunia, tobacco PR-1a promoter, ubiquitin promoter, actin promoter, alcA gene promoter, pin2 promoter (Xu et al., 1993), maize WipI promoter, maize trpA gene promoter (U.S. Pat. No. 5,625,136), maize CDPK gene promoter, and RUBISCO SSU promoter (U.S. Pat. No. 5,034,322) can also be used. Tissue-specific promoters, for example fruit-specific promoters, such as the E8 promoter of tomato (accession number: AF515784; Good et al. (1994)) can be used. Fruit-specific promoters such as flower organ-specific promoters can be used with an expression construct of the present invention for expressing a polynucleotide of the invention in the flower organ of a plant. Examples of flower organ-specific promoters include any of the promoter sequences described in U.S. Pat. Nos. 6,462,185; 5,639,948; and 5,589,610. Seed-specific promoters such as the promoter from a B-phaseolin gene (for example, of kidney bean) or a glycinin gene (for example, of soybean), and others, can also be used. Endosperm-specific promoters include, but are not limited to, MEG1 (EPO application No. EP1528104) and those described by Wu et al. (1998), Furtado et al. (2002), and Hwang et al. (2002). Root-specific promoters, such as any of the promoter sequences described in U.S. Pat. No. 6,455,760 or U.S. Pat. No. 6,696,623, or in published U.S. patents application Ser. Nos. 20/040,078841; 20040067506; 20040019934; 20030177536; 20030084486; or 20040123349, can be used with an expression construct of the invention.

Expression constructs of the invention may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides of the invention. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

DNA sequences which direct polyadenylation of mRNA transcribed from the expression construct can also be included in the expression construct, and include, but are not limited to, an octopine synthase or nopaline synthase signal. The expression constructs of the invention can also include a polynucleotide sequence that directs transposition of other genes, i.e., a transposon.

Polynucleotides of the present invention can be composed of either RNA or DNA. Preferably, the polynucleotides are composed of DNA. In one embodiment, the DNA is complementary DNA (cDNA) prepared from or based on a messenger RNA (mRNA) template sequence. The subject invention also encompasses those polynucleotides that are complementary in sequence to the polynucleotides disclosed herein. Polynucleotides and polypeptides of the invention can be provided in purified or isolated form.

Because of the degeneracy of the genetic code, a variety of different polynucleotide sequences can encode polypeptides and enzymes of the present invention. A table showing all possible triplet codons (and where U also stands for T) and the amino acid encoded by each codon is described in Lewin (1985). In addition, it is well within the skill of a person trained in the art to create alternative polynucleotide sequences encoding the same, or essentially the same, polypeptides and enzymes of the subject invention. These variant or alternative polynucleotide sequences are within the scope of the subject invention. As used herein, references to “essentially the same” sequence refers to sequences which encode amino acid substitutions, deletions, additions, or insertions which do not materially alter the functional activity of the polypeptide encoded by the polynucleotides of the present invention. Allelic variants of the nucleotide sequences encoding a wild type polypeptide of the invention are also encompassed within the scope of the invention.

Substitution of amino acids other than those specifically exemplified or naturally present in a wild type polypeptide or enzyme of the invention are also contemplated within the scope of the present invention. For example, non-natural amino acids can be substituted for the amino acids of a polypeptide, so long as the polypeptide having the substituted amino acids retains substantially the same biological or functional activity as the polypeptide in which amino acids have not been substituted. Examples of non-natural amino acids include, but are not limited to, ornithine, citrulline, hydroxyproline, homoserine, phenylglycine, taurine, iodotyrosine, 2,4-diaminobutyric acid, α-amino isobutyric acid, 4-aminobutyric acid, 2-amino butyric acid, γ-amino butyric acid, ε-amino hexanoic acid, 6-amino hexanoic acid, 2-amino isobutyric acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form. Allelic variants of a protein sequence of a wild type polypeptide or enzyme of the present invention are also encompassed within the scope of the invention.

Amino acids can be generally categorized in the following classes: non-polar, uncharged polar, basic, and acidic. Conservative substitutions whereby a polypeptide or enzyme of the present invention having an amino acid of one class is replaced with another amino acid of the same class fall within the scope of the subject invention so long as the polypeptide having the substitution still retains substantially the same biological or functional activity (e.g., enzymatic) as the polypeptide that does not have the substitution. Polynucleotides encoding a polypeptide or enzyme having one or more amino acid substitutions in the sequence are contemplated within the scope of the present invention. Table 4 provides a listing of examples of amino acids belonging to each class.

TABLE 4

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

The subject invention also concerns variants of the polynucleotides of the present invention that encode functional polypeptides of the invention. Variant sequences include those sequences wherein one or more nucleotides of the sequence have been substituted, deleted, and/or inserted. The nucleotides that can be substituted for natural nucleotides of DNA have a base moiety that can include, but is not limited to, inosine, 5-fluorouracil, 5-bromouracil, hypoxanthine, 1-methylguanine, 5-methylcytosine, and tritylated bases. The sugar moiety of the nucleotide in a sequence can also be modified and includes, but is not limited to, arabinose, xylulose, and hexose. In addition, the adenine, cytosine, guanine, thymine, and uracil bases of the nucleotides can be modified with acetyl, methyl, and/or thio groups. Sequences containing nucleotide substitutions, deletions, and/or insertions can be prepared and tested using standard techniques known in the art.

Fragments and variants of a polypeptide or enzyme of the present invention can be generated as described herein and tested for the presence of biological or enzymatic function using standard techniques known in the art. Thus, an ordinarily skilled artisan can readily prepare and test fragments and variants of a polypeptide or enzyme of the invention and determine whether the fragment or variant retains functional or biological activity (e.g., enzymatic activity) relative to full-length or a non-variant polypeptide.

Polynucleotides and polypeptides contemplated within the scope of the subject invention can also be defined in terms of more particular identity and/or similarity ranges with those sequences of the invention specifically exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

As used herein, the terms “nucleic acid” and “polynucleotide” refer to a deoxyribonucleotide, ribonucleotide, or a mixed deoxyribonucleotide and ribonucleotide polymer in either single-or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. Allelic variations of the exemplified sequences also fall within the scope of the subject invention. The polynucleotide sequence includes both the sense and antisense strands either as individual strands or in the duplex.

Techniques for transforming plant cells with a polynucleotide or gene are known in the art and include, for example, Agrobacterium infection, transient uptake and gene expression in plant seedlings, biolistic methods, electroporation, calcium chloride treatment, PEG-mediated transformation, etc. U.S. Pat. No. 5,661,017 teaches methods and materials for transforming an algal cell with a heterologous polynucleotide. Transformed cells can be selected, redifferentiated, and grown into plants that contain and express a polynucleotide of the invention using standard methods known in the art. The seeds and other plant tissue and progeny of any transformed or transgenic plant cells or plants of the invention are also included within the scope of the present invention. In one embodiment, the cell is transformed with a polynucleotide sequence comprising a sequence encoding the amino acid sequence shown in any of SEQ ID NOs: 2, 4, 6, 8, 10, 12, 14, 16, 18, 20, 22, 24, 26, 28, 30, 32, 34, or 36, or an enzymatically active fragment or variant thereof.

The subject invention also concerns cells transformed with a polynucleotide of the present invention encoding a polypeptide or enzyme of the invention. In one embodiment, the cell is transformed with a polynucleotide sequence comprising a sequence encoding the amino acid sequence shown in any of SEQ ID NOs: 2, 4, 6, 8, 10, 12, 14, 16, 18, 20, 22, 24, 26, 28, 30, 32, 34, or 36, or an enzymatically active fragment or variant thereof. In one embodiment, the polynucleotide is stably incorporated into the genome of the cell. In another embodiment, the polynucleotide is not incorporated into the cell genome and is transiently expressed. In one embodiment, the polynucleotide sequence of the invention is provided in an expression construct of the invention. The transformed cell can be a prokaryotic cell, for example, a bacterial cell such as E. coli or B. subtilis, or the transformed cell can be a eukaryotic cell, for example, a plant cell, including protoplasts, or an animal cell. Plant cells include, but are not limited to, dicotyledonous, monocotyledonous, and conifer cells. Animal cells include human cells, mammalian cells, avian cells, and insect cells. Mammalian cells include, but are not limited to, COS, 3T3, and CHO cells. Cells of the invention can be grown in vitro, e.g., in a bioreactor or in tissue culture. Cells of the invention can also be grown in vivo, e.g., as ascites in a mammal, in a seed of a plant (such as corn or soybean seeds), etc.

Single letter amino acid abbreviations are defined in Table 5.

TABLE 5

| Letter Symbol | Amino Acid |
|---|---|
| A | Alanine |
| B | Asparagine or |
| | aspartic acid |
| C | Cysteine |
| D | Aspartic Acid |
| E | Glutamic Acid |
| F | Phenylalanine |
| G | Glycine |
| H | Histidine |
| I | Isoleucine |
| K | Lysine |
| L | Leucine |
| M | Methionine |
| N | Asparagine |
| P | Proline |
| Q | Glutamine |
| R | Arginine |
| S | Serine |
| T | Threonine |
| V | Valine |
| W | Tryptophan |
| Y | Tyrosine |
| Z | Glutamine or |
| | glutamic acid |

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Produce SGSH and SGSH-Lectin Fusion Proteins

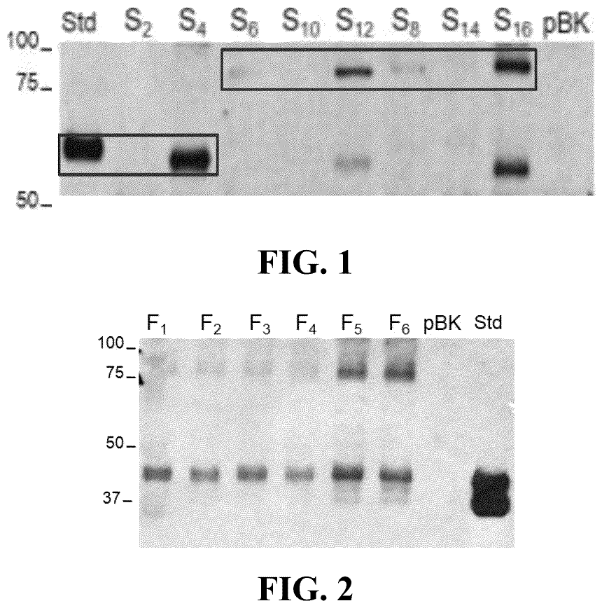

Construct design and plant-based expression. Sixteen gene constructs encoding SGSH and SGSH fusions with RTB and NBB (Table 6) were developed and expressed transiently in N. benthamiana leaves. Variants assessing signal peptides (human SGSH vs. plant-derived signal peptide), codon usage (SGSH sequence vs tobacco codon optimized), and fusion orientation were compared for product yield and quality (FIG. 1). Constructs were introduced into Agrobacterium tumefaciens, and induced cultures were vacuum infiltrated into leaves of intact plants and incubated for 2 to 5 days prior to harvest (Medrano et al., 2009). All constructs produced recombinant products of the expected sizes (56 kDa for SGSH; ˜91 kDa for lectin-SGSH fusions) that cross-reacted with anti-RTB, anti-His-tag, and anti-SGSH antibodies as appropriate (e.g., see FIG. 1). All constructs that used the native human signal peptide showed significantly lower product than those using the BioStrategies' plant signal peptide (PoSP). Expression kinetics in planta indicated abundant product at 48 and 72 h post-infiltration indicating product stability. FIG. 1 compares protein yields of selected constructs. For lectin-SGSH fusions (S5-S16), PoSP and lectin fused at the C-terminus (S12 for RTB and S16 for NBB) gave better protein yields (although some cleavage between the domains was observed with this orientation, the amount of full length protein is higher than lectin fused at the N-terminus). Based on these results, we selected a construct harboring SGSH (S4), SGSH-RTB fusion (S12) and SGSH-NBB fusion (S16) for further studies in Examples 2 and 3.

TABLE 6
Sulfamidase and lectin fusion constructs

| | | | | SGSH | | | | | | | | |
|---|---|---|---|---|---|---|---|---|---|---|---|---|
| | | Signal peptide | | N-term | | C-term | | RTBtr | | NBB | | His |
| Construct | Abbr. | SGSH | PoSP | Nat | Opt | Nat | Opt | N-term | C-term | N-term | C-term | tag |
| hSP:SGSH^{NAT}:His | S1 | X | | X | | | | | | | | X |
| (nucleotide: SEQ ID NO: 38) | | | | | | | | | | | | |
| (amino acid: SEQ ID NO: 39) | | | | | | | | | | | | |
| hSP:SGSH^{OPT}:His | S2 | X | | | X | | | | | | | X |
| (nucleotide: SEQ ID NO: 40) | | | | | | | | | | | | |
| (amino acid: SEQ ID NO: 41) | | | | | | | | | | | | |
| PoSP:SGSH^{NAT}:His | S3 | | X | X | | | | | | | | X |
| (nucleotide: SEQ ID NO: 42) | | | | | | | | | | | | |
| (amino acid: SEQ ID NO: 43) | | | | | | | | | | | | |
| PoSP:SGSH^{OPT}:His | S4 | | X | | X | | | | | | | X |
| (nucleotide: SEQ ID NO: 44) | | | | | | | | | | | | |
| (amino acid: SEQ ID NO: 45) | | | | | | | | | | | | |
| PoSP:RTBtr:SGSH^{NAT}:His | S5 | | X | | | X | | X | | | | X |
| (nucleotide: SEQ ID NO: 46) | | | | | | | | | | | | |
| (amino acid: SEQ ID NO: 47) | | | | | | | | | | | | |
| PoSP:RTBtr:SGSH^{OPT}:His | S6 | | X | | | | X | X | | | | X |
| (nucleotide: SEQ ID NO: 48) | | | | | | | | | | | | |
| (amino acid: SEQ ID NO: 49) | | | | | | | | | | | | |
| PoSP:NBB:SGSH^{NAT}:His | S7 | | X | | | X | | | | X | | X |
| (nucleotide: SEQ ID NO: 50) | | | | | | | | | | | | |
| (amino acid: SEQ ID NO: 51) | | | | | | | | | | | | |
| PoSP:NBB:SGSH^{OPT}:His | S8 | | X | | | | X | | | X | | X |
| (nucleotide: SEQ ID NO: 52) | | | | | | | | | | | | |
| (amino acid: SEQ ID NO: 53) | | | | | | | | | | | | |
| hSP:SGSH^{NAT}:RTBtr:His | S9 | X | | X | | | | | X | | | X |
| (nucleotide: SEQ ID NO: 54) | | | | | | | | | | | | |
| (amino acid: SEQ ID NO: 55) | | | | | | | | | | | | |
| hSP:SGSH^{OPT}:RTBtr:His | S10 | X | | | X | | | | X | | | X |
| (nucleotide: SEQ ID NO: 56) | | | | | | | | | | | | |
| (amino acid: SEQ ID NO: 57) | | | | | | | | | | | | |
| PoSP:SGSH^{NAT}:RTBtr:His | Sil | | X | X | | | | | X | | | X |
| (nucleotide: SEQ ID NO: 58) | | | | | | | | | | | | |
| (amino acid: SEQ ID NO: 59) | | | | | | | | | | | | |
| PoSP:SGSH^{OPT}:RTBtr:His | S12 | | X | | X | | | | X | | | X |
| (nucleotide: SEQ ID NO: 60) | | | | | | | | | | | | |
| (amino acid: SEQ ID NO: 61) | | | | | | | | | | | | |
| hSP:SGSH^{NAT}:NBB:His | S13 | X | | X | | | | | | | X | X |
| (nucleotide: SEQ ID NO: 62) | | | | | | | | | | | | |
| (amino acid: SEQ ID NO: 63) | | | | | | | | | | | | |
| hSP:SGSH^{OPT}:NBB:His | S14 | X | | | X | | | | | | X | X |
| (nucleotide: SEQ ID NO: 64) | | | | | | | | | | | | |
| (amino acid: SEQ ID NO: 65) | | | | | | | | | | | | |
| PoSP:SGSH^{NAT}:NBB:His | S15 | | X | X | | | | | | | X | X |
| (nucleotide: SEQ ID NO: 66) | | | | | | | | | | | | |
| (amino acid: SEQ ID NO: 67) | | | | | | | | | | | | |
| PoSP:SGSH^{OPT}:NBB:His | S16 | | X | | X | | | | | | X | X |
| (nucleotide: SEQ ID NO: 68) | | | | | | | | | | | | |
| (amino acid: SEQ ID NO: 69) | | | | | | | | | | | | |

Constructs harboring only SGSH were considered as located at the N-term in this table.
PoSP, Patatin Optimized Signal Peptide/
Nat, native sequence/
Opt, codon optimized sequence based on Nicotiana tabacum/
N-term, N terminus/
C-term, C terminus/
His tag, 6x histidine tag

TABLE 7
Sulfatase modifying factor 1 (FGE)

| | | Signal peptide | | SUMF1 | | His | |
|---|---|---|---|---|---|---|---|
| Construct | Abbr. | SUMF1 | PoSP | Nat | Opt | tag | KDEL |
| hSP:SUMF1^{NAT}:His | F1 | X | | X | | X | |
| (nucleotide: SEQ ID NO: 70) | | | | | | | |
| (amino acid: SEQ ID NO: 71) | | | | | | | |
| hSP:SUMF1^{OPT}:His | F2 | X | | | X | X | |
| (nucleotide: SEQ ID NO: 72) | | | | | | | |
| (amino acid: SEQ ID NO: 73) | | | | | | | |
| PoSP:SUMF1^{NAT}:His | F3 | | X | X | | X | |
| (nucleotide: SEQ ID NO: 74) | | | | | | | |
| (amino acid: SEQ ID NO: 75) | | | | | | | |
| PoSP:SUMF1^{OPT}:His | F4 | | X | | X | X | |
| (nucleotide: SEQ ID NO: 76) | | | | | | | |
| (amino acid: SEQ ID NO: 77) | | | | | | | |
| PoSP:SUMF1^{NAT}:His:KDEL | F5 | | X | X | | X | X |
| (nucleotide: SEQ ID NO: 78) | | | | | | | |
| (amino acid: SEQ ID NO: 79) | | | | | | | |
| PoSP:SUMF1^{OPT}:His:KDEL | F6 | | X | | X | X | X |
| (nucleotide: SEQ ID NO: 80) | | | | | | | |
| (amino acid: SEQ ID NO: 81) | | | | | | | |

PoSP, Patatin Optimized Signal Peptide
Nat, native sequence
Opt, codon optimized sequence based on Nicotiana tabacum
His tag, 6x histidine tag
KDEL, KDEL retrieval sequence (SEQ ID NO: 82)

### EXAMPLE 2

### Assess SGSH Enzyme and Carbohydrate-Binding Activity of Plant-Made SGSH and SGSH-Lectin Fusions

Assessment of SGSH activity. Plant tissues expressing S4, S12 and S16 constructs were used for extraction and initial purification of the SGSH and SGSH-fusion proteins. Several extraction buffers and clarification strategies were tested with the goal to obtain initial test material to assess activity. Leaf extracts were subjected to an initial affinity chromatography enrichment step (Nickel IMAC was used for the His-tagged S4; lactose resin for the S12 RTB fusion, and N-acetyl-galactosamine resin for the S16 NBB fusion). Recovery of the S12 and S16 products on selective sugar affinity columns confirmed lectin activity of the products. These proteins were quantified and used to assess SGSH activity based on the standard 2-step fluorometric assay as described (Karpova et. al., 1996) and using recombinant human SGSH (Novoprotein; made in HEK293 cells) as control proteins. No sulfamidase activity was detected in the plant-derived products.

SUMF1. Sulfatases carry a unique amino acid in their active site, Cα-formylglycine (FGly), which is required for their catalytic activity. In this reaction, a specific cysteine is oxidized to FGly by the formylglycine-generating enzyme (FGE), as a post/co-translational modification that happens in nascent sulfatase polypeptides within the endoplasmic reticulum in mammalian cells. FGE is encoded by the sulfatase modifying factor 1 (SUMF1) gene. Phylogenetic studies have not identified SUMF1 homologs in plants and plants do not contain sulfatases that contain this modification. To support co-expression studies, we developed six new constructs for expression of human SUMF1 (Table 7). Native cDNA sequence encoding human SUMF1 (NCBI NM_182760) and tobacco-codon optimized SUMF1 cDNA were synthesized (GENEART) to include a C-term hexahistidine tag. Two signal peptides were tested (SUMF1 SP vs our plant PoSP). In addition, constructs adding a C-terminal KDEL ER retrieval sequence (SEQ ID NO:82) were developed. SUMF1 acts on SGSH in the ER; its ER-localization is mediated by a region within the N-terminus (residues 34-68; Malalyalam et al., 2008). This retention mechanism does not appear highly effective in animal cells (significant amounts of SUMF1 are secreted) and the ability of plants to “read” this ER signal was unknown. We therefore produced a KDEL-modified version to ensure ER retention of SUMF1 in plants. SUMF1 constructs (Table 7) were expressed transiently in N. benthamiana leaves and yields were assessed at 48, 72, and 96 hr post-infiltration. All constructs produced recombinant products of the expected sizes (42 kDa) that cross-reacted with anti-SUMF1 (FIG. 2) and anti-His antibodies. The highest expression of SUMF1 was at 72 h post-infiltration; codon optimization and signal peptide did not have significant impact on protein yield. However, the KDEL signal (SEQ ID NO:82) appears to enhance protein stability; SUMF1-KDEL remained at high levels at both 72 and 96 hr. F6 was selected for initial co-expression studies.

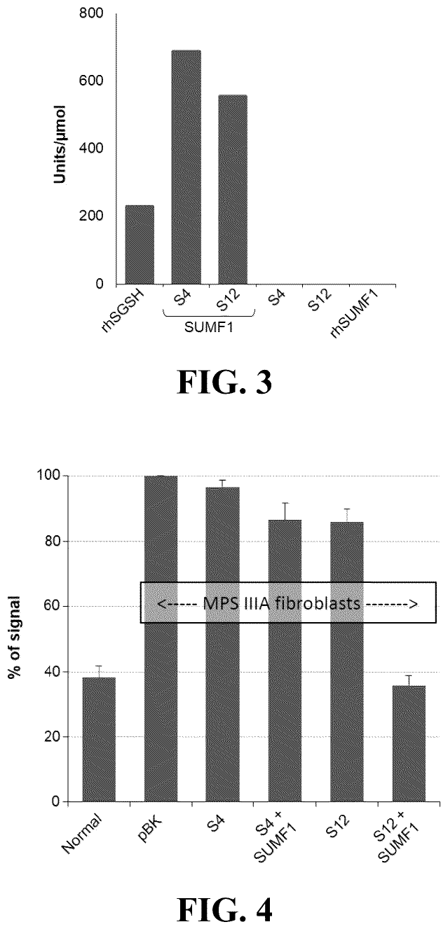

SUMF1/SGSH co-expression yields active sulfamidase. In order to determine if SUMF1 mediated formylglycine modification of SGSH in plants leading to production of an enzymatically active sulfatase, leaves were infiltrated with a 1:1 mixed culture of Agrobacterium tumefaciens (“Agro”) harboring SUMF1 (F6) and SGSH (S4 or S12). Leaves were harvested at 72 h post-infiltration and purified by affinity chromatography, as described above for S4 and S12 constructs. Mammalian cell-derived SGSH and plant-derived SGSH (S4) and SGSH-RTB (S12) that were expressed in the presence or absence of SUMF1 (F6) were tested for sulfamidase enzymatic activity (FIG. 3) and shown as units/μmol to encompass differences in molecular size of each protein. As shown in FIG. 3, plant-made SGSH (S4 and S12) were enzymatically active only when SUMF1 was co-expressed, and were more active than SGSH made in HEK293 human cells. SGSH:NBB (S16) showed analogous SGSH activity when expressed with SUMF1 (not shown). For the S12 product, protein identity (both SGSH and RTB) and FGly modification were confirmed through peptide sequencing by mass spectrometry (MS/MS; UAMS Biomedical Research Center). FGly modification was only found when SGSH was co-expressed with SUMF1. Our results demonstrate plants can produce fully active SGSH when co-expressed with SUMF1 and that the lectin fusion partner does not inhibit enzyme activity. Interestingly, co-expression with SUMF1-KDEL provided greater SGSH product yields than un-modified forms (not shown) suggesting broader applications using other production platforms or for gene therapy approaches for the entire sulfatase family.

### EXAMPLE 3

### Demonstrate Uptake, Lysosomal Delivery, and Reduction of “Disease Substrate” in MPS IIIA Cultured Cells Treated with SGSH and SGSH-Lectin Fusions

MPS IIIA patients are deficient in SGSH activity leading to pathological accumulation of sulfated glucosaminoglycans (GAGs) with cellular phenotypes including elevated GAGs and increased lysosomal volume per cell. As a further demonstration that the plant-produced SGSH was fully functional following modification by SUMF1, MPS IIIA patient fibroblasts (GM01881) were treated with plant-produced SGSH (S4) or SGSH-RTB (S12) that were expressed in the presence and absence of co-expressed SUMF1 (FIG. 4). S12 (SGSH:RTB) produced in the presence of SUMF1 effectively reduced GAG content and lysosomal volume to “normal” levels. SGSH alone (S4+/−SUMF1) was not corrective indicating that lectin-based delivery as well as FGly activation are critical in phenotype correction. These results indicate that RTB effectively delivers active SGSH to the site of GAG disease substrate accumulation resulting in disease phenotype correction at the cellular level. Analogous results have been demonstrated with S16 (SGSH:NBB; co-expressed with SUMF1; data not shown) indicating that multiple plant lectins can facilitate cellular uptake and lysosomal delivery of plant-made sulfatases.

### EXAMPLE 4

### Increase Sulfatase Activity by Modifying Co-Expression Parameters of SGSH and SUMF1

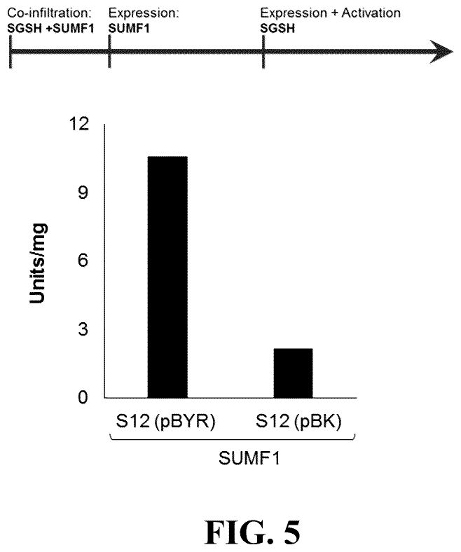

SUMF1 is localized to the ER and acts on mammalian sulfatases as they are co-translationally inserted into rough ER. The strategy for plant based SGSH and SUMF1 production in Example 2 and Example 3 involved co-expression where the kinetics of expression were the same and demonstrated that the SGSH enzymatic activity directly reflects the FGly modification mediated by SUMF1. Strategies that differentially change the kinetics of either SGSH or SUMF1 production such that SUMF1 is present in the plant ER prior and during the production phase for SGSH may result in a greater efficiency of SGSH modification and provide a higher specific activity product. Two strategies were selected for testing this (among many that could be used including expressing SGSH and SUMF1 under control of differentially expressed promoters, transiently expressing SGSH in a stable transgenic plant engineered to constitutively express the SUMF1 transgene, and other strategies providing SUMF1 activity prior and during production of the sulfatase). First, the S12 (SGSH:RTB) gene was introduced into a plant Agro/viral vectoring system (pBYR) (Huang et al., 2009) which is typically infiltrated at lower levels with a delayed initiation of high-level expression. Agro cultures bearing SUMF1 in the pBK (NCBI GU982971) vector were co-infiltrated with Agro strains bearing either S12 (SGSH: RTB) in pBK or in the Agro/viral vector pBYR. SGSH activity was then assessed in protein purified from leaf extracts and compared to the recombinant human SGSH produced in human HEK293 cells. The specific activity of plant-derived S12 product produced using the mixed Agro (SUMF1) and Agro/viral (S12 SGSH: RTB) co-expression parameters was 3-5 fold higher than S12 produced using a 1:1 ratio of the same genes both expressed using the pBK Agro vectoring system (shown in FIG. 5). The specific activity of the product was also 6-9 fold more active than human cell-derived rhSGSH indicating higher levels of FGly modification.

A second demonstration that directed expression such that onset of SUMF1 production occurs earlier than SGSH increases the yield of active SGSH was also shown. In this example, a culture of Agro bearing the SUMF1 gene construct (F6) were induced by treatment with acetosyringone for 24 hours. Acetosyringone speeds activation of bacterial virulence leading to faster expression of transfected recombinant proteins. The induced culture was then mixed with Agro cultures bearing the S12 (SGSH: RTB) construct that was not induced. The mixed culture was then vacuum infiltrated into leaves of intact N. benthamiana plants and the plants were incubated for 3 to 5 days and harvested. The S12 sulfatase enzyme activity was 2-fold higher under conditions where the SUMF1-bearing Agro was selectively pre-induced by acetosyringone compared to previous infiltrations where both the SUMF1 and S12 strains were simultaneously activated by acetosyrongone. These results indicate that having plant cells “pre-loaded” with the SUMF1 modifying protein yields S12 wherein a significantly greater proportion of the SGSH product is modified to its fully functional form. By modifying the temporal parameters for expression (through either induction or viral vectoring systems), we demonstrated that the plant-based system yields sulfatase product with significantly greater specific activity (higher FGly modification) than the commercially available products produced in human cells (as much as 9-fold greater).

### EXAMPLE 5

### Demonstrate in Vivo Efficacy of SUMF1-Activated Sulfatase Enzyme Replacement Therapeutics by Treating Sulfatase-Deficient Mice

The biochemical and behavioral aspects of disease development have been well-characterized in the SGSH-deficient MPS-IIIA mouse model. These mice show elevated heparan sulfate levels detected at birth, lysosomal vacuolarization evident by 3-6 weeks of age and progressively worsening of behavioral/cognitive problems (altered activity, aggression, gait dysfunction, learning deficits, leading to lethargy and death) (Crawley et al., 2006). To assess in vivo efficacy of the S12 (SGSH:RTB) and S16 (SGSH:NBB) these fusion products are synthesized in plants that are co-expressing SUMF1 or SUMF1 modified with KDEL (SEQ ID NO:82) and purified to at least 95% purity of sulfatase enzyme activity with endotoxin levels below that recommended for mouse trials. MPS IIIA mice (e.g., 6-8 week old mice) are treated with the plant-derived S12 or S16 fusion product by i.v.-administration in doses ranging from 1 to 5 mg/kg in MPS-IIIA mice and analysis is done by methods similar to those described previously for this disease model (Rozaklis et al., 2011) and compared with wild type and untreated MPS IIIA control mice. For short-term biodistribution analyses, genotype-confirmed MPS-IIIA mice and unaffected −/+ or +/+are treated (i.v., tail vein) with 100-150 μl ‘vehicle’ (PBS), S12, or S16. At 1, 2, and 3 hr, serum is collected by orbital bleed from 3 mice/group to determine serum clearance of the product. At 4, 12, and 24 hr after injection, 3 mice/time point (MPS-IIIA and WT mice) are euthanized, serum collected (heart puncture) and liver, spleen, and brain tissues are either formalin fixed or snap-frozen in liquid nitrogen for subsequent analyses. SGSH levels and enzyme activity is measured in tissues and serum as described (Rozaklis et al., 2011). Presence of the S12 and S16 products in specific tissues is confirmed by immunohistochemistry of fixed tissue.

To demonstrate efficacy in reducing GAG levels (the MPS IIIA disease substrate) and correcting the tissue pathology (e.g., cellular vacuolization; accumulation of associated gangliosides), MPS IIIA mice are treated 1-2 times per week with doses ranging from 0.5 to 5 mg/kg body weight for 4-6 weeks and the mice are harvested to assess SGSH levels, GAG levels and cellular morphology in selected tissues (e.g., liver, kidney, and multiple tissues of the brain). To demonstrate impacts on behavior of this neurodegenerative disease, weekly treatment can be extended to a total of 12-16 weeks and assessment of behavioral aspects are performed by open-field tests measuring activity and rearing behaviors (MPS-IIIA mice display reduced activity/gait) and memory/learning tests (e.g., using a Morris water maze). At study endpoint (72 hr after final injection), mice are euthanized and blood collected by heart puncture. Some animals from each group are fixation-perfused and processed for histological analyses. For biochemical analyses, livers, spleens and brains of non-perfused animals are sliced and frozen for heparan sulfate analyses. Immunohistological analyses include assessment of neuronal pathology in the cerebral cortex and hippocampus (e.g., using LIMP-II and GM3 as markers which are significantly elevated in untreated MPS IIIA mice). Extended administration of the S12 and S16 fusions is expected to lead to increased sulfatase activity, decreased GAG levels, and improvement in cellular phenotype and behavior in the MPS IIIA mice.

### EXAMPLE 6

### Demonstrate in Vivo Efficacy of SUMF1 Enzyme Replacement Therapeutics in Treating the SUMF1^{-/-} Mouse Model for Multiple Sulfatase Deficiency

Similar to Example 5, plant-made SUMF1 fusions are used as an enzyme replacement therapy for treating SUMF1^{-/-} mice. This mouse model shows similar disease development as multiple sulfatase deficiency patients (Settembre et al., 2007). Specifically, SUMF1^{-/-} mice show growth retardation and skeletal abnormalities, neurological defects, and early mortality. At the cellular level, there is significant vacuolization, lysosomal storage of glycosaminoglycans, and inflammatory responses characterized by abundant highly vacuolated macrophages. For these studies, SUMF1 fusions are produced in plants and purified to greater than 95% enzyme purity with acceptable endotoxin levels. These products may include: RTB:SUMF1, NBB:SUMF1, RTB:SUMF1-KDEL, NBB:SUMF1-KDEL, SUMF1:RTB-KDEL or SUMF1:NBB-KDEL with the lectin providing uptake and the KDEL (SEQ ID NO:82) or SUMF1 domains directing subcellular trafficking to the ER. SUMF1 fusions are administered to mice and serum and tissues processed as previously described in Example 5. In addition, tissues are assayed for sulfatase activity (which is totally lacking in this mutant mouse strain due to absence of SUMF1). Extended administration of the SUMF1-lectin fusions results in increased sulfatase activity, decreased GAG levels, and improvement in macrophage morphology and disease phenotype.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 4,938,949
U.S. Pat. No. 5,034,322
U.S. Pat. No. 5,106,739
U.S. Pat. No. 5,589,610
U.S. Pat. No. 5,625,136
U.S. Pat. No. 5,639,948
U.S. Pat. No. 5,661,017
U.S. Pat. No. 5,929,304
U.S. Pat. No. 6,455,760
U.S. Pat. No. 6,462,185
U.S. Pat. No. 6,696,623
U.S. Published Application No. 20030084486
U.S. Published Application No. 20030177536
U.S. Published Application No. 20040019934
U.S. Published Application No. 20040067506
U.S. Published Application No. 20040078841
U.S. Published Application No. 20040123349
European Application No. EP1528104
Altschul, S. F. et al. (1990) “Basic Local Alignment Search Tool” J. Mol. Biol. 215:402-410.
Altschul, S. F. et al. (1997) “Gapped BLAST and PSI-BLAST: A New Generation of Protein Database Search Programs” Nucl. Acids Res. 25:3389-3402.
Chen et al. (2010) BioTechniques 49:513-518.
Clancy, M. and Hannah, L.C. (2002) “Splicing of the maize Sh1 first intron is essential for enhancement of gene expression, and a T-rich motif increases expression without affecting splicing” Plant Physiol. 130(2):918-29.
Furtado, A. et al. (2002) “Tools for Use in the Genetic Engineering of Barley” Proceedings of the 10^{th} Australian Barley Technical Symposium, Canberra, ACT, Australia.
Good, X. et al. (1994) “Reduced ethylene synthesis by transgenic tomatoes expressing S-adenosylmethionine hydrolase” Plant Molec. Biol. 26:781-790.
Hwang, Y-S. et al. (2002) “Analysis of the Rice Endosperm-Specific Globulin Promoter in Transformed Rice Cells” Plant Cell Rep. 20:842-847.
Karlin S. and Altschul, S. F. (1990) “Methods for Assessing the Statistical Significance of Molecular Sequence Features by Using General Scoring Schemes” Proc. Natl. Acad. Sci. USA 87:2264-2268.
Karlin S. and Altschul, S. F. (1993) “Applications and Statistics for Multiple High-Scoring Segments in Molecular Sequences” Proc. Natl. Acad. Sci. USA 90:5873-5877.
Lewin, B. (1985) Genes II, John Wiley & Sons, Inc., p. 96.
Lovrinovic and Niemeyer (2005) BBRC 335:943-948.
Lungwitz et al. (2005) Eur. J. Pharmacet. Bioparmacet. 60:247-266.
Wu, C-L. et al. (1998) “Promoters of Rice Seed Storage Protein Genes Direct Endosperm-Specific Gene Expression in Transgenic Rice” Plant and Cell Physiology, 39(8):885-889.
Xu, D., McElroy, D., Thornburg, R. W., Wu, R. et al. (1993) “Systemic induction of a potato pin2 promoter by wounding, methyl jasmonate, and abscisic acid in transgenic rice plants” Plant Molecular Biology 22:573-588.
Van Damme et al. “Characterization and molecular cloning of Sambucus nigra agglutinin V (nigrin b), a GalNAc-specific type-2 ribosome-inactivating protein from the bark of elderberry (Sambucus nigra)” Eur. J. Biochem. 237 (2), 505-513 (1996).
Maveyraud et al. “Structural basis for sugar recognition, including the tn carcinoma antigen, by the lectin sna-ii from sambucus nigra” Proteins 75 p.89 (2009).
Van de Kamp et al. “Genetic heterogeneity and clinical variability in the Sanfilippo syndrome (type A, B, and C)” Clin. Genet. 20 (2), 152-160 (1981).
Montfort et al. “The three-dimensional structure of ricin at 2.8A” J. Biol Chem. 262 (11), 5398-5403 (1987).
Citores L, Munoz R, Rojo MA, Jimenez P, Ferreras JM, Girbes T (2003) Cell. Molec. Biol. 49:461-465.
Citores L, Munoz R, De Benito FM, Iglesias R, Ferreras JM, Girbes T (1996) Cell. Molec. Biol. 42(4):473-476.
Ferreras et al., (2011) Toxins 3:420-441.
Sandvig K, van Deurs B (1999) FEBS Lett 452(1-2):67-70.
Simmons BM, Stahl PD, Russell JH (1986) J Biol Chem 261(17):7912-7920.
Van Damme et al., (1998) Crit. Rev. Plant Sci. 17:575-692.
Bevan et al. “The structure and transcription start site of a major potato tuber protein gene” Nucleic Acid Res. 14 (11), 4625-4638 (1986).
Malalyalam et al., 2008
Huang Z, Chen Q, Hjelm B, Arntzen C, Mason H. A DNA replicon system for rapid high-level production of virus-like particle in plants. Biotechnol Bioeng, 2009, 103(4): 706-714.
Crawley AC, Gliddon BL, Auclair D, Brodie SL, Hirte C, King BM, Fuller M, Hemsley KM, Hopwood JJ. Characterization of a C57BL/6 congenic mouse strain of mucopolysaccharidosis type IIIA. Brain Res, 2006, 1104(1):1-17.
Settembre C, Annunziata I, Spampanato C, Zarcone, D, Cobellis G, Nusco E, Zito E, Tacchetti C, Cosma MP, Ballabio A. 2007. Sytemic inflammation and neurodegeneration of a mouse model of multiple sulfatase deficiency. Proc. Natl. Acad. Sci USA, 2007, 104:4506-11.
1. Meikle PJ, Hopwood JJ, Clague AE, Carey WF: Prevalence of lysosomal storage disorders. JAMA 1999, 281(3):249-254.
2. Hollak CE, Aerts JM, Ayme S, Manuel J: Limitations of drug registries to evaluate orphan medicinal products for the treatment of lysosomal storage disorders. Orphanet J Rare Dis 2011, 6:16.
3. Grabowski GA: Treatment perspectives for the lysosomal storage diseases. Expert Opin Emerg Drugs 2008, 13(1):197-211.
4. Du H, Cameron TL, Garger SJ, Pogue GP, Hamm LA, White E, Hanley KM, Grabowski GA: Wolman disease/cholesteryl ester storage disease: efficacy of plant-produced human lysosomal acid lipase in mice. J Lipid Res 2008, 49(8):1646-1657.
5. Aviczer D, Brill-Almon E, Shaaltiel Y, Hashmueli S, Bartfeld D, Mizrachi S, Liberman Y, Freeman A, Zimran A, Galun E: A plant-derived recombinant human glucocerebrosidase enzyme-a preclinical and phase I investigation. PLoS One 2009, 4(3):e4792.
6. Zimran A, Brill-Almon E, Chertkoff R, Petakov M, Blanco-Favela F, Munoz ET, Solorio-Meza SE, Amato D, Duran G, Giona F et al: Pivotal trial with plant cell-expressed recombinant glucocerebrosidase, taliglucerase alfa, a novel enzyme replacement therapy for Gaucher disease. Blood 2011, 118(22):5767-5773.
7. Pastores G, Shankar SP, Szer J, Petakov M, Cox TM, Giraldo P, Rosenbaum H, Amato DJ, Mengel E, Chertkoff R et al: Plant cell-expressed recombinant glucocerebrosidase: Taliglucerase alfa as therapy for Gaucher disease in adults patients previously treated with imiglucerase: 24-month results. Mol Genet Metab 2013, 108(2):S73-S74.
8. Medrano G, Reidy M, Liu J, Ayala J, Dolan M, Cramer C: Rapid system for evaluating bioproduction capacity of complex pharmaceutical proteins in plants. Methods Mol Biol 2009, 483:51-67.
9. Huang Z, Phoolcharoen W, Lai H, Piensook K, Cardincau G, Zeitlin L, Whaley KJ, Arntzen CJ, Mason HS, Chen Q: High-level rapid production of full-size monoclonal antibodies in plants by a single-vector DNA replicon system. Biotechnol Bioeng 2010, 106(1):9-17.
10. D'Aoust MA, Couture MM, Charland N, Trepanier S, Landry N, Ors F, Vezina LP: The production of hemagglutinin-based virus-like particles in plants: a rapid, efficient and safe response to pandemic influenza. Plant Biotechnol J 2010, 8(5):607-619.
11. Whaley KJ, Hiatt A, Zeitlin L: Emerging antibody products and Nicotiana manufacturing. Hum Vaccines 2011, 7(3):349-356.
12. Komarova TV, Baschieri S, Donini M, Marusic C, Benvenuto E, Dorokhov YL: Transient expression systems for plant-derived biopharmaceuticals. Expert Rev Vaccines 2010, 9(8):859-876.
13. Lai H, Chen Q: Bioprocessing of plant-derived virus-like particles of Norwalk virus capsid protein under current Good Manufacture Practice regulations. Plant Cell Rep 2012, 31(3):573-584.
14. Landry N, Ward BJ, Trepanier S, Montomoli E, Dargis M, Lapini G, Vezina LP: Preclinical and clinical development of plant-made virus-like particle vaccine against avian H5N1 influenza. PLoS One 2010, 5(12):e15559.
15. Landis SC, Amara SG, Asadullah K, Austin CP, Blumenstein R, Bradley EW, Crystal RG, Darnell RB, Ferrante RJ, Fillit H et al: A call for transparent reporting to optimize the predictive value of preclinical research. Nature 2012, 490(7419):187-191.

16. Sandvig K, van Deurs B: Endocytosis and intracellular transport of ricin: recent discoveries. FEBS Lett 1999, 452(1-2):67-70.
17. Jackman MR, Shurety W, Ellis JA, Luzio JP: Inhibition of apical but not basolateral endocytosis of ricin and folate in Caco-2 cells by cytochalasin D. J Cell Sci 1994, 107 (Pt 9):2547-2556.
18. Frankel A, Fu T, Burbage C, Tagge E, Harris B, Vesely J, Willingham M: Lectin-deficient ricin toxin intoxicates cells bearing the D-mannose receptor. Carbohyd Res 1997, 300(3):251-258.
19. Simmons BM, Stahl PD, Russell JH: Mannose receptor-mediated uptake of ricin toxin and ricin A chain by macrophages. Multiple intracellular pathways for a chain translocation. J Biol Chem 1986, 261(17):7912-7920.
20. Morlon-Guyot J, Helmy M, Lombard-Frasca S, Pignol D, Pieroni G, Beaumelle B: Identification of the ricin lipase site and implication in cytotoxicity. J Biol Chem 2003, 278(19):17006-17011.
21. Stechmann B, Bai S, Gobbo E, Lopez R, Merer G, Pinchard S, Panigai L, Tenza D, Raposo G, Beaumelle B et al: Inhibition of retrograde transport protects mice from lethal ricin challenge. Cell 2010, 141(2):231-242.
22. Choi N, Estes M, Langridge W: Mucosal immunization with a ricin toxin B subunit-rotavirus NSP4 fusion protein stimulates a Th1 lymphocyte response. J Biotechnol 2006, 121(2):272-283.
23. Donayre-Torres A, Esquivel-Soto E, Gutiérrez-Xicoténcatl ML, Esquivel-Guadarrama F, Gómez-Lim M: Production and purification of immunologically active core protein p24 from HIV-1 fused to ricin toxin B subunit in E. coli. Virol J 2009, 6:17.
24. Medina-Bolivar F, Wright R, Funk V, Sentz D, Barroso L, Wilkins T, Petri WJ, Cramer C: A non-toxic lectin for antigen delivery of plant-based mucosal vaccines. Vaccine 2003, 21(9-10):997-1005.
25. Cramer CL, Reidy M, Dolan MC: Methods of delivery of molecules to cells using ricin subunit and compositions relating to same. US Published Application No. 2010/0240597.
26. Reidy MJ: Engineering of the RTB lectin as a carrier platform for proteins and antigens. Blacksburg, VA: PhD Dissertation, Virginia Polytechnic Institute and Arkansas State University; 2007.
27. Liu J: Plant-derived murine IL-12 and ricin b-murine IL-12 fusions. Blacksburg, VA: PhD Dissertation, Virginia Polytechnic Institute and Arkansas State University; 2006.
28. Citores L, Munoz R, Rojo MA, Jimenez P, Ferreras JM, Girbes T: Evidence for distinct cellular internalization pathways of ricin and nigrin b. Cell Mol Biol 2003, 49 Online Pub: OL461-465.
29. Battelli MG, Citores L, Buonamici L, Ferreras JM, de Benito FM, Stirpe F, Girbes T: Toxicity and cytotoxicity of nigrin b, a two-chain ribosome-inactivating protein from Sambucus nigra: comparison with ricin. Arch Toxicol 1997, 71(6):360-364.
30. Citores L, Munoz R, De Benito FM, Iglesias R, Ferreras JM, Girbes T: Differential sensitivity of HELA cells to the type 2 ribosome-inactivating proteins ebulin 1, nigrin b and nigrin f as compared with ricin. Cell Mol Biol 1996, 42(4):473-476.
31. Zhang Y, Pardridge WM: Delivery of beta-galactosidase to mouse brain via the blood-brain barrier transferrin receptor. J Pharmacol Exp Ther 2005, 313(3):1075-1081.
32. Begley DJ, Pontikis CC, Scarpa M: Lysosomal storage diseases and the blood-brain barrier. Curr Pharm Design 2008, 14(16):1566-1580.
33. Audi J, Belson M, Patel M, Schier J, Osterloh J: Ricin poisoning: a comprehensive review. JAMA 2005, 294(18):2342-2351.
34. Broadwell RD, Balin BJ, Salcman M: Transcytotic pathway for blood-borne protein through the blood-brain barrier. Proc Natl Acad Sci USA 1988, 85(2):632-636.
35. Thorne RG, Emory CR, Ala TA, Frey WH, 2nd: Quantitative analysis of the olfactory pathway for drug delivery to the brain. Brain Res 1995, 692(1-2):278-282.
36. Bell CL, Gurda BL, Van Vliet K, Agbandje-McKenna M, Wilson JM: Identification of the galactose binding domain of the adeno-associated virus serotype 9 capsid. J Virol 2012, 86(13):7326-7333.
37. Shen S, Bryant KD, Brown SM, Randell SH, Asokan A: Terminal N-linked galactose is the primary receptor for adeno-associated virus 9. J Biol Chem 2011, 286(15):13532-13540.
38. Trickler WJ, Lantz SM, Murdock RC, Schrand AM, Robinson BL, Newport GD, Schlager JJ, Oldenburg SJ, Paule MG, Slikker W, Jr. et al: Silver nanoparticle induced blood-brain barrier inflammation and increased permeability in primary rat brain microvessel endothelial cells. Toxicol Sci 2010, 118(1):160-170.
39. Bachmeier CJ, Trickler WJ, Miller DW: Comparison of drug efflux transport kinetics in various blood-brain barrier models. Drug Metab Dispos 2006, 34(6):998-1003.
40. Tessitore A, del PMM, Sano R, Ma Y, Mann L, Ingrassia A, Laywell ED, Steindler DA, Hendershot LM, d'Azzo A: GM1-ganglioside-mediated activation of the unfolded protein response causes neuronal death in a neurodegenerative gangliosidosis. Mol Cell 2004, 15(5):753-766.
41. Crawley AC, Gliddon BL, Auclair D, Brodic SL, Hirte C, King BM, Fuller M, Hemsley KM, Hopwood JJ: Characterization of a C57BL/6 congenic mouse strain of mucopolysaccharidosis type IIIA. Brain Res 2006, 1104(1):1-17.
42. Gliddon BL, Hopwood JJ: Enzyme-replacement therapy from birth delays the development of behavior and learning problems in mucopolysaccharidosis type IIIA mice. Pediatr Res 2004, 56(1):65-72.
43. Polito VA, Abbondante S, Polishchuk RS, Nusco E, Salvia R, Cosma MP: Correction of CNS defects in the MPSII mouse model via systemic enzyme replacement therapy. Hum Mol Genet 2010, 19(24):4871-4885.
44. Grubb JH, Vogler C, Tan Y, Shah GN, MacRac AF, Sly WS: Infused Fc-tagged beta-glucuronidase crosses the placenta and produces clearance of storage in utero in mucopolysaccharidosis VII mice. Proc Natl Acad Sci USA 2008, 105(24):8375-8380.
45. Vogler C, Levy B, Grubb JH, Galvin N, Tan Y, Kakkis E, Pavloff N, Sly WS: Overcoming the blood-brain barrier with high-dose enzyme replacement therapy in murine mucopolysaccharidosis VII. Proc Natl Acad Sci USA 2005, 102(41):14777-14782.
46. Blanz J, Stroobants S, Lullmann-Rauch R, Morelle W, Ludemann M, D'Hooge R, Reuterwall H, Michalski JC, Fogh J, Andersson C et al: Reversal of peripheral and central neural storage and ataxia after recombinant enzyme replacement therapy in alpha-mannosidosis mice. Hum Mol Genet 2008, 17(22):3437-3445.
47. Matzner U, Lullmann-Rauch R, Stroobants S, Andersson C, Weigelt C, Eistrup C, Fogh J, D'Hooge R, Gieselmann V: Enzyme replacement improves ataxic gait and central nervous system histopathology in a mouse model of metachromatic leukodystrophy. Mol Ther 2009, 17(4):600-606.
48. Rozaklis T, Beard H, Hassiotis S, Garcia AR, Tonini M, Luck A, Pan J, Lamsa JC, Hopwood JJ, Hemsley KM: Impact of high-dose, chemically modified sulfamidase on pathology in a murine model of MPS IIIA. Exp Neurol 2011, 230(1):123-130.
49. Hemsley KM, Beard H, King BM, Hopwood JJ: Effect of high dose, repeated intra-cerebrospinal fluid injection of sulphamidase on neuropathology in MPS IIIA mice. Genes Brain Behav 2008, 7:740-753.
50. Hemsley KM, Luck AJ, Crawley AC, Hassiotis S, Beard H, King B, Rozek T, Rozaklis T, Fuller M, Hopwood JJ: Examination of intravenous and intra-CSF protein delivery for treatment of neurological disease. Eur J Neurosci 2009, 29(6):1197-1214.
51. Fraldi A, Hemsley K, Crawley A, Lombardi A, Lau A, Sutherland L, Auricchio A, Ballabio A, Hopwood JJ: Functional correction of CNS lesions in an MPS-IIIA mouse model by intracerebral AAV-mediated delivery of sulfamidase and SUMF1 genes. Hum Mol Genet 2007, 16(22):2693-2702.
52. Sorrentino NC, D'Orsi L, Sambri I, Nusco E, Monaco C, Spampanato C, Polishchuk E, Saccone P, De Leonibus E, Ballabio A et al: A highly secreted sulphamidase engineered to cross the blood-brain barrier corrects brain lesions of mice with mucopolysaccharidoses type IIIA. EMBO Mol Med 2013, 5(5):675-690.
53. Karpova EA, Voznyi Ya V, Keulemans JL, Hoogeveen AT, Winchester B, Tsvetkova IV, van Diggelen OP: A fluorimetric enzyme assay for the diagnosis of Sanfilippo disease type A (MPS IIIA). J Inherit Metab Dis 1996, 19(3):278-285.
54. Landgrebe J, Dierks T, Schmidt B, von Figura K: The human SUMF1 gene, required for posttranslational sulfatase modification, defines a new gene family which is conserved from pro-to eukaryotes. Gene 2003, 316:47-56.
55. Mariappan M, Gande SL, Radhakrishnan K, Schmidt B, Dierks T, von Figura K: The non-catalytic N-terminal extension of formylglycine-generating enzyme is required for its biological activity and retention in the endoplasmic reticulum. J Biol Chem 2008, 283(17):11556-11564.
56. Diez-Roux G, Ballabio A: Sulfatases and human disease. Annu Rev Genom Hum Genet 2005, 6:355-379.
57. Cosma MP, Pepe S, Annunziata I, Newbold RF, Grompe M, Parenti G, Ballabio A: The multiple sulfatase deficiency gene encodes an essential and limiting factor for the activity of sulfatases. Cell 2003, 113(4):445-456.
58. Zito E, Buono M, Pepe S, Settembre C, Annunziata I, Surace EM, Dierks T, Monti M, Cozzolino M, Pucci P et al: Sulfatase modifying factor 1 trafficking through the cells: from endoplasmic reticulum to the endoplasmic reticulum. EMBO J 2007, 26(10):2443-2453.
59. Sardiello M, Annunziata I, Roma G, Ballabio A: Sulfatases and sulfatase modifying factors: an exclusive and promiscuous relationship. Hum Mol Genet 2005, 14(21):3203-3217.
60. Annunziata I, Bouche V, Lombardi A, Settembre C, Ballabio A: Multiple sulfatase deficiency is due to hypomorphic mutations of the SUMF1 gene. Hum Mutat 2007, 28(9):928.
61. Cosma MP, Pepe S, Parenti G, Settembre C, Annunziata I, Wade-Martins R, Di Domenico C, Di Natale P, Mankad A, Cox B et al: Molecular and functional analysis of SUMF1 mutations in multiple sulfatase deficiency. Hum Mutat 2004, 23(6):576-581.
62. McCullen CA, Binns AN: Agrobacterium tumefaciens and plant cell interactions and activities required for interkingdom macromolecular transfer. Annu Rev Cell Dev Biol 2006, 22:101-127.
63. Acosta-Gamboa W: Development of plant lectin RTB for delivery of therapeutic proteins. Jonesboro, AR: PhD dissertation. Arkansas State University; 2012.
64. Huynh HT, Grubb JH, Vogler C, Sly WS: Biochemical evidence for superior correction of neuronal storage by chemically modified enzyme in murine mucopolysaccharidosis VII. Proc Natl Acad Sci USA 2012, 109(42):17022-17027.
65. Hemsley KM, King B, Hopwood JJ: Injection of recombinant human sulfamidase into the CSF via the cerebellomedullary cistern in MPS IIIA mice. Mol Genet Metab 2007, 90(3):313-328.
66. Trim PJ, Lau AA, Hopwood JJ, Snel MF: A simple method for early age phenotype confirmation using toe tissue from a mouse model of MPS IIIA. Rapid Commun Mass Spectrom 2014, 28(8):933-938.
67. Whitfield PD, Nelson P, Sharp PC, Bindloss CA, Dean C, Ravenscroft EM, Fong BA, Fietz MJ, Hopwood JJ, Meikle PJ: Correlation among genotype, phenotype, and biochemical markers in Gaucher disease: implications for the prediction of disease severity. Mol Genet Metab 2002, 75(1):46-55.
68. Boado RJ, Hui EK, Lu JZ, Zhou QH, Pardridge WM: Reversal of lysosomal storage in brain of adult MPS-I mice with intravenous Trojan horse-iduronidase fusion protein. Mol Pharm 2011, 8(4):1342-1350.
69. Grabowski GA: Perspectives on gene therapy for lysosomal storage diseases that affect hematopoiesis. Curr Hematol Rep 2003, 2(4):356-362.
70. Beck M: Therapy for lysosomal storage disorders. IUBMB Life 2010, 62(1):33-40.
71. Hemsley KM, Norman EJ, Crawley AC, Auclair D, King B, Fuller M, Lang DL, Dean CJ, Jolly RD, Hopwood JJ: Effect of cisternal sulfamidase delivery in MPS IIIA Huntaway dogs-a proof of principle study. Mol Genet Metab 2009, 98(4):383-392.
72. Smallshaw JE, Vitetta ES: Ricin vaccine development. Curr Top Microbiol Immunol 2012, 357:259-272.
73. Yermakova A, Mantis NJ: Protective immunity to ricin toxin conferred by antibodies against the toxin's binding subunit (RTB). Vaccine 2011, 29(45):7925-7935.
74. Rayon C, Lerouge P, Faye L: The protein N-glycosylation in plants. J Exp Bot 1998, 49(326):1463-1472.
75. Chargelegue D, Vine ND, van Dolleweerd CJ, Drake PM, Ma JK: A murine monoclonal antibody produced in transgenic plants with plant-specific glycans is not immunogenic in mice. Transgenic Res 2000, 9(3):187-194.

I claim:

1. A fusion protein comprising i) a mammalian sulfatase, or an enzymatically active fragment or variant thereof, and ii) a plant lectin or a binding subunit thereof; or a fusion protein comprising i) a mammalian sulfatase modifying factor 1 (SUMF1), or an enzymatically active fragment or variant thereof, and ii) a plant lectin or a binding subunit thereof; or a polynucleotide encoding said fusion protein; or
a human SUMF1 protein expressed in plant cells comprising transforming a plant cell with an expression vector comprising a nucleotide sequence for translational expression of the SUMF1 protein linked to a signal sequence directing expression; or a mammalian sulfatase, or an enzymatically active fragment or variant thereof, produced in a plant or plant cell.

2. The fusion protein according to claim 1, wherein the mammalian sulfatase is N-acetylgalactosamine-6-sulfatase, N-acetylglucosamine-6-sulfatase, N-sulphoglucosamine sulphohydrolase, sulfamidase, extracellular sulfatase Sulf-1 (hSulf1), extracellular sulfatase Sulf-2 (hSulf2), iduronate 2-sulfatase, arylsulfatase A (ASA), arylsulfatase B (ASB), steryl-sulfatase, arylsulfatase D (ASD), arylsulfatase E (ASE), arylsulfatase F (ASF), arylsulfatase G (ASG), arylsulfatase H (ASH), arylsulfatase I (ASI), arylsulfatase J (ASJ), or arylsulfatase K (ASK).

3. The fusion protein according to claim 1, wherein the mammalian sulfatase comprises the amino acid sequence of SEQ ID NOs: 2, 4, 6, 8, 10, 12, 14, 16, 18, 20, 22, 24, 26, 28, 30, 32, or 34, or an enzymatically active fragment or variant thereof.

4. The fusion protein according to claim 1, wherein the plant lectin is a lectin from Table 2 or Table 3 of the specification.

5. The fusion protein according to claim 1, wherein the plant lectin is the non-toxic subunit of ricin (RTB) or nigrin (NBB).

6. The fusion protein according to claim 1, wherein the fusion protein comprises an endoplasmic reticulum (ER) retention sequence.

7. The fusion protein according to claim 6, wherein the ER retention sequence comprises KDEL.

8. The fusion protein according to claim 1, wherein the mammalian sulfatase is linked to the plant lectin by a linker sequence of amino acids.

9. The fusion protein according to claim 1, wherein the SUMF1 comprises the amino acid sequence of SEQ ID NO:36, or an enzymatically active fragment or variant thereof.

10. The human SUMF1 protein of claim 1, wherein the SUMF1 protein shows enzymatic and biological activity with capacity to activate human sulfatases expressed in plant cells.

11. The human SUMF1 protein of claim 10, wherein the SUMF1 protein activates a sulfatase in plant cells by catalyzing the conversion of a relevant cysteine to a FGly residue required for activating enzymatic activity of the sulfatase.

12. The fusion protein according to claim 1, wherein the fusion protein comprises the amino acid sequence of any of SEQ ID NOs: 39, 41, 43, 45, 47, 49, 51, 53, 55, 57, 59, 61, 63, 65, 67, or 69, or an enzymatically active fragment or variant thereof; or wherein the fusion protein comprises the amino acid sequence of any of SEQ ID NOs: 71, 73, 75, 77, 79, or 81, or an enzymatically active fragment or variant thereof.

13. A method for treating or preventing a disease or condition associated with a sulfatase enzyme or a sulfatase modifying factor 1 (SUMF1) protein in a person or animal, comprising administering to the person or animal a therapeutically effective amount of a SUMF1 protein or a fusion protein having sulfatase activity, and/or an enzymatically active mammalian sulfatase protein having sulfatase activity, comprising co-expressing in a plant or plant cell: (1) a polynucleotide encoding a mammalian sulfatase fusion protein, and/or a polynucleotide encoding a mammalian sulfatase protein; and (2) a polynucleotide encoding a mammalian sulfatase modifying factor 1 (SUMF1) protein or a SUMF1 fusion protein, wherein the expressed sulfatase fusion protein and/or the expressed mammalian sulfatase protein produced by the method is enzymatically activated in the plant or plant cell by the expressed SUMF1 protein or the expressed SUMF1 fusion protein.


SEQUENCE LISTING (82 sequences)

SEQ ID NO: 1 - DNA, 2380 bp, Homo sapiens
  source 1..2380: mol_type = genomic DNA
aggccccgccccgcagcccagccggaagggccggcggacgctcgctaggtcggctcgctggccggggctccgcggctcccgtggttgccatggcggcggttgtcgcggcgacgaggtggtggcagctgttgctggtgctcagcgccgcggggatgggggcctcgggcgccccgcagccccccaacatcctgctcctgctcatggacgacatgggatggggtgacctcggggtgtatggagagccctccagagagaccccgaatttggaccggatggctgcagaagggctgcttttcccaaacttctattctgccaaccctctgtgctcgccatcgagggcggcactgctcacaggacggctacccatccgcaatggcttctacaccaccaacgcccatgccagaaacgcctacacaccgcaggagattgtgggcggcatcccagactcggagcagctcctgccggagcttctgaagaaggccggctacgtcagcaagattgtcggcaagtggcatctgggtcacaggccccagttccaccccctgaagcacggatttgatgagtggtttggatcccccaactgccactttggaccttatgacaacaaggccaggcccaacatccctgtgtacagggactgggagatggttggcagatattatgaagaatttcctattaatctgaagacgggggaagccaacctcacccagatctacctgcaggaagccctggacttcattaagagacaggcacggcaccacccctttttcctctactgggctgtcgacgccacgcacgcacccgtctatgcctccaaacccttcttgggcaccagtcagcgagggcggtatggagacgccgtccgggagattgatgacagcattgggaagatactggagctcctccaagacctgcacgtcgcggacaacaccttcgtcttcttcacgtcggacaacggcgctgccctcatttccgcccccgaacaaggtggcagcaacggcccctttctgtgtgggaagcagaccacgtttgaaggagggatgagggagcctgccctcgcatggtggccagggcacgtcactgcaggccaggtgagccaccagctgggcagcatcatggacctcttcaccaccagcctggcccttgcgggcctgacgccgcccagcgacagggccattgatggcctcaacctcctccccaccctcctgcagggccggctgatggacaggcctatcttctattaccgtggcgacacgctgatggcggccaccctcgggcagcacaaggctcacttctggacctggaccaactcctgggagaacttcagacagggcattgatttctgccctgggcagaacgtttcaggggtcacaactcacaatctggaagaccacacgaagctgcccctgatcttccacctgggacgggacccaggggagaggttccccctcagctttgccagcgccgagtaccaggaggccctcagcaggatcacctcggtcgtccagcagcaccaggaggccttggtccccgcgcagccccagctcaacgtgtgcaactgggcggtcatgaactgggcacctccgggctgtgaaaagttagggaagtgtctgacacctccagaatccattcccaagaagtgcctctggtcccactagcacctgcgcagactcaggccaggcctagaatctccggttggccctgcaagtgcctggaggaaggatggctctggcctcggtcctcccccaaccctgcccaagccagacagacagcacctgcagacgcagggggactgcacaattccacctgcccaggacctgaccctggcgtgtgcttggccctcctcctcgcccacggcgcctcagatttcaggaccctcctcctcgcccacggcgcctcagacctcaggaccctgccgtctcacgcctttgtgaaccccaaatatctgagaccagtctcagtttattttgccaaggttaaggatgcacctgtgacagcctcaggaggtcctgacaacaggtgcctgaggtggctggggatacagtttgcctttatacatcttagggagacacaagatcagtatgtgtatggcgtacattggttcagtcagccttccactgaatacacgattgagtctggcccagtgaatccgcatttttatgtaaacagtaagggaacggggcaatcatataagcgtttgtctcaggggagccccagagggatgacttccagttccgtctgtcctttgtccacaaggaatttccctggacgctaattatgagggaggcgtgtagcttcttatcattgtaactatgttatttagaaataaaacgggaggcaggtttgcctaattcccagcttgaaaaaaaaaaaaaaaaaa

SEQ ID NO: 2 - protein, 522 aa, Homo sapiens
MAAVVAATRWWQLLLVLSAAGMGASGAPQPPNILLLLMDDMGWGDLGVYGEPSRETPNLDRMAAEGLLFPNFYSANPLCSPSRAALLTGRLPIRNGFYTTNAHARNAYTPQEIVGGIPDSEQLLPELLKKAGYVSKIVGKWHLGHRPQFHPLKHGFDEWFGSPNCHFGPYDNKARPNIPVYRDWEMVGRYYEEFPINLKTGEANLTQIYLQEALDFIKRQARHHPFFLYWAVDATHAPVYASKPFLGTSQRGRYGDAVREIDDSIGKILELLQDLHVADNTFVFFTSDNGAALISAPEQGGSNGPFLCGKQTTFEGGMREPALAWWPGHVTAGQVSHQLGSIMDLFTTSLALAGLTPPSDRAIDGLNLLPTLLQGRLMDRPIFYYRGDTLMAATLGQHKAHFWTWTNSWENFRQGIDFCPGQNVSGVTTHNLEDHTKLPLIFHLGRDPGERFPLSFASAEYQEALSRITSVVQQHQEALVPAQPQLNVCNWAVMNWAPPGCEKLGKCLTPPESIPKKCLWSH

SEQ ID NO: 3 - DNA, 5144 bp, Homo sapiens
  source 1..5144: mol_type = genomic DNA
ccctgtccctggctcacgtgatcgcgcctagggagaaaacgtctgactccagccaccggccttcaaggcacggctttttattccttcggctggtcggcctctcgcccttcagctacctgtgcgtccctccgtcccgtcccgtcccggggtcaccccggagcctgtccgctatgcggctcctgcctctagccccaggtcggctccggcggggcagcccccgccacctgccctcctgcagcccagcgctgctactgctggtgctgggcggctgcctgggggtcttcggggtggctgcgggaacccggaggcccaacgtggtgctgctcctcacggacgaccaggacgaagtgctcggcggcatgacaccgctaaagaaaaccaaagctctcatcggagagatggggatgactttttccagtgcttatgtgccaagtgctctctgctgccccagcagagccagtatcctgacaggaaagtacccacataatcatcacgttgtgaacaacactctggaggggaactgcagtagtaagtcctggcagaagatccaagaaccaaatactttcccagcaattctcagatcaatgtgtggttatcagaccttttttgcagggaaatatttaaatgagtacggagccccagatgcaggtggactagaacacgttcctctgggttggagttactggtatgccttggaaaagaattctaagtattataattacaccctgtctatcaatgggaaggcacggaagcatggtgaaaactatagtgtggactacctgacagatgttttggctaatgtctccttggactttctggactacaagtccaactttgagcccttcttcatgatgatcgccactccagcgcctcattcgccttggacagctgcacctcagtaccagaaggctttccagaatgtctttgcaccaagaaacaagaacttcaacatccatggaacgaacaagcactggttaattaggcaagccaagactccaatgactaattcttcaatacagtttttagataatgcatttaggaaaaggtggcaaactctcctctcagttgatgaccttgtggagaaactggtcaagaggctggagttcactggggagctcaacaacacttacatcttctatacctcagacaatggctatcacacaggacagttttccttgccaatagacaagagacagctgtatgagtttgatatcaaagttccactgttggttcgaggacctgggatcaaaccaaatcagacaagcaagatgctggttgccaacattgacttgggtcctactattttggacattgctggctacgacctaaataagacacagatggatgggatgtccttattgcccattttgagaggtgccagtaacttgacctggcgatcagatgtcctggtggaataccaaggagaaggccgtaacgtcactgacccaacatgcccttccctgagtcctggcgtatctcaatgcttcccagactgtgtatgtgaagatgcttataacaatacctatgcctgtgtgaggacaatgtcagcattgtggaatttgcagtattgcgagtttgatgaccaggaggtgtttgtagaagtctataatctgactgcagacccagaccagatcactaacattgctaaaaccatagacccagagcttttaggaaagatgaactatcggttaatgatgttacagtcctgttctgggccaacctgtcgcactccaggggtttttgaccccggatacaggtttgacccccgtctcatgttcagcaatcgcggcagtgtcaggactcgaagattttccaaacatcttctgtagcgacctcacacagcctctgcagatggatccctgcacgcctctttctgatgaagtgattgtagtaggtgtctgtagctagtcttcaagaccacacctggaagagtttctgggctggctttaagtcctgtttgaaaaagcaacccagtcagctgacttcctcgtgcaatgtgttaaactgtgaactctgcccatgtgtcaggagtggctgtctctggtctcttcctttagctgacaaggacactcctgaggtctttgttctcactgtattctttttatcctggggccacagttcttgattattcctcttgtggttaaagactgaatttgtaaacccattcagataaatggcagtactttaggacacacacaaacacacagacacaccttttgatatgtaagcttgacctaaagtcaaaggacctgtgtagcatttcagattgagcacttcactatcaaaaatactaacatcacatggcttgaagagtaaccatcagagctgaatcatccaagtaagaacaagtaccattgttgattgataagtagagatacattttttatgatgttcatcacagtgtggtaaggttgcaaattcaaaacatgtcacccaagctctgttcatgtttttgtgaattctaggctggtgctgcactgaaatagagcagtaagcttgtgataaaggccaattccaggtagctcttgaaggtgatagccatctactttccagtggctgccaaccacagggagtgccagttaacactggaaggattaaggcaaggtcccttctcttgagactcccctctgagatctgaaaaatgaagtggcttaggaacatcagcagtgaagaactgccaagagttggtgaaggttgtctcttccgagggccttctgaagacagggctcttgaacagacaagtggaagggctgtaccagggataaaggaaagaagtgcctgtccagcagggagcttgaatttaagttccatgtatgaagtcattggctctatctgcatttttctgtcattctcttcatttgttttaaggtggaaaattttcttacagttgatgcaaagtatcaactactttaccctaccttctccccttttagatgggttcttcctgagttttggagtcttgtatgattatcagtattcccctgtcaaaatcaaatctattcaggtttcttcactgttgagaacacctaaatgtttttatttttgagaagtggggacagagtctcactatgtcacccaggctggagtgcaatggcatgatctcagctcactgcaaccttcgcctcctgggttcaagcgattctcctgcctccgcctcctgagtagctgggattataggcacgcaccaccacgcccagctaattttttgtatttttagtagagacagagtttcaccatgttggccaggctggtcttgaactcctgaccttgtgatccacccacctcggcctcccagagtgctgggattacaggcatgagccaccacgcttggctaagaacacctaaatttttatgtttcttggctcaaaaaccagttccatttctaatgttgtcctcacaagaaggctaattggtggtgagacagcaggggaggaggaagagctgtggtttgtaacttgttcaactcaggcaataagcgattttagctttatttaaagtcttctgtccagctttaagcactttgtaagacatggctgaaagtagcttttctatcagaattgcagatagtcatgttgggctaacagtcaattggatatattcctttacctcacatgaccccagcaactgtggtggtatctagaggtgaaacaggcaagtgaaatggacacctctgctgtgaatgttttagagaaggaaattcaaaaaatgttgtaactgaaagcactgttgaatatgggtatcggctttctttttcactttgactcttaacattatcagtcaacttccacattaatgaaagttgaccatagttatttccaaataaaaagaaaccaactcttaccaggtcttggactgtgatgtcatattattcagttttatgcttgttcctgagcagaactcataagagtgacatagtcagctgctgacggcacctcagccacgccactcttactcagttcagtgggtgtgcttgcgtggtaggatgtggtgcagccctctctacgctcttctatttttggtatatttcctatctaaccttcaaatagcttccaattctttttttcttggactggcttcattctgaatttgtgctaaaataatctttcataaagagacctcagtttatagcgtaacagactacacaatgcactgatgttttcataatgtttaagggacccactgcaagaagcttgctgcctccttttaattgtattcatttagattttgattttccatgttaagaaggtgaggtccatgttggtgcccttcagagtagagaaccatgtaaacattaggaatgaacagaggccttaggaatgaatagagagtttgccttatacaatttcctgttacaaagctctccctctcatgcaaagtagggaacaccttttgagcatctttgaatttgacaaatggtgctgttgcaaacactttttttttgagatgaagtctcgcggttgtcacccgggctggagtgcagtggcgtgatctcggctcactgcaacttccacctcctgggttccagcagttctcctgcctcagcctcccaagtagctgagattacaggcgcctgccaccccacctggctgatttttgtaattttagtagagacggggtttcaccatgttggccaggctgattaactcctgacctcaggtgatccacctttctcggcctcccaaagtgctgggattacgggtgtgagccaccgtgcccggcctgcaaacacattttaattgacaacactagggctgttgtacaaaatagtaatgatagccatggaagttttaccttattctgtgagaagtgttcttaaacttattaagtgtctaaactaaggtttagtgcttttttaaaggaaagttgtcccaggattcatcctaaagaaagcaaaagttaattcaactgatccaccaatggaattagatgggtagagttgggttcttgagttttaccaccacttagttcccactgaattttgtaacttcctgtgtttgcatcctctgttcctattctgcccttgctctgtgtcatctcagtcatttgacttagaaagtgcccttcaaaaggaccctgttcactgctgcacttttcaatgaattaaaatttatttctgttctagtgggaaaaaaaaaaaaaaaaaa

SEQ ID NO: 4 - protein, 552 aa, Homo sapiens
MRLLPLAPGRLRRGSPRHLPSCSPALLLLVLGGCLGVFGVAAGTRRPNVVLLLTDDQDEVLGGMTPLKKTKALIGEMGMTFSSAYVPSALCCPSRASILTGKYPHNHHVVNNTLEGNCSSKSWQKIQEPNTFPAILRSMCGYQTFFAGKYLNEYGAPDAGGLEHVPLGWSYWYALEKNSKYYNYTLSINGKARKHGENYSVDYLTDVLANVSLDFLDYKSNFEPFFMMIATPAPHSPWTAAPQYQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKPNQTSKMLVANIDLGPTILDIAGYDLNKTQMDGMSLLPILRGASNLTWRSDVLVEYQGEGRNVTDPTCPSLSPGVSQCFPDCVCEDAYNNTYACVRTMSALWNLQYCEFDDQEVFVEVYNLTADPDQITNIAKTIDPELLGKMNYRLMMLQSCSGPTCRTPGVFDPGYRFDPRLMFSNRGSVRTRRFSKHLL

SEQ ID NO: 5 - DNA, 2770 bp, Homo sapiens
  source 1..2770: mol_type = genomic DNA
gaccggggtcggggcagggggcggggccgagcgggagaccagagagccggagccggatcccgatcccgagtccgagccgccgccgccatgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtgaccatcccaggaggcctgtgcacacatcccaggcatgtcccagacacatcccacacgtgtccgtgtggccggccagcctggggagtagtggcaacagcccttccgtccacactcccatccaaggagggttcttccttcctgtggggtcactcttgccattgcctggagggggaccagagcatgtgaccagagcatgtgcccagcccctccaccaccaggggcactgccgtcatggcaggggacacagttgtccttgtgtctgaaccatgtcccagcacgggaattctagacatacgtggtctgcggacagggcagcgcccccagcccatgacaagggagtcttgttttctggcttggtttggggacctgcaaatgggaggcctgaggccctcttcaggctttggcagccacagatacttctgaacccttcacagagagcaggcaggggcttcggtgccgcgtgggcagtacgcaggtcccaccgacactcacctgggagcacggcgcctggctcttaccagcgtctggcctagaggaagcctttgagcgacctttgggcaggtttctgcttcttctgttttgccccatggtcaagtccctgttccccaggcaggtttcagctgattggcagcaggctccctgagtgatgagcttgaacctgtggtgtttctgggcagaagcttatcttttttgagagtgtccgaagatgaaggcatggcgatgcccgtcctctggcttgggttaattcttcggtgacactggcattgctgggtggtgatgcccgtcctctggcttgggttaattcttcggtgacactggcgttgctgggtggcaatgcccatcctctgccttgggttaattcttcggtgacactggcgttgctgggtggcgatgcccgtcctctggcttgggttaattcttggatgacgtcggcgttgctgggagaatgtgccgttcctgccctgcctccacccacctcgggagcagaagcccggcctggacacccctcggcctggacacccctcgaaggagagggcgcttccttgagtaggtgggctccccttgcccttccctccctatcactccatactggggtgggctggaggaggccacaggccagctattgtaaaagctttttattttagtaaaatatacagaagttctttttctgaaaa

SEQ ID NO: 6 - protein, 502 aa, Homo sapiens
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 7 - DNA, 5710 bp, Homo sapiens
  source 1..5710: mol_type = genomic DNA
aggttacttgactgggagttctcagacctccagtttcagccctgccctcagcctccaatccgtaagagacacccagccccagcaattggattgggcagcccgtcttgacacaccactgtgctgagtgcttgaggacgtgtttcaacagatggttggggttagtgtgtgtcatcacattcgagtggggattaagagaaggaaggctgccttgctggagctgtgtggtcttctccaagtgagagtcgcaggcaatagaactactttgcttttggaggaaaaggaggaattcattttcagcagacacaagaaaagcagtttttttttcagggattcttcacttctcttgaacaaggaactcactcagagactaacacaaaggaagtaatttcttacctggtcattatttagtctacaataagttcatccttcttcagtgtgaccagtaaattcttcccatactcttgaagagagcataattggaatggagaggtgctgacggccacccaccatcatctaaagaagataaacttggcaaatgacatgcaggttcttcaaggcagaataattgcagaaaatcttcaaaggaccctatctgcagatgttctgaatacctctgagaatagagattgattattcaaccaggatacctaattcaagaactccagaaatcaggagacggagacattttgtcagttttgcaacattggaccaaatacaatgaagtattcttgctgtgctctggttttggctgtcctgggcacagaattgctgggaagcctctgttcgactgtcagatccccgaggttcagaggacggatacagcaggaacgaaaaaacatccgacccaacattattcttgtgcttaccgatgatcaagatgtggagctggggtccctgcaagtcatgaacaaaacgagaaagattatggaacatgggggggccaccttcatcaatgcctttgtgactacacccatgtgctgcccgtcacggtcctccatgctcaccgggaagtatgtgcacaatcacaatgtctacaccaacaacgagaactgctcttccccctcgtggcaggccatgcatgagcctcggacttttgctgtatatcttaacaacactggctacagaacagccttttttggaaaatacctcaatgaatataatggcagctacatcccccctgggtggcgagaatggcttggattaatcaagaattctcgcttctataattacactgtttgtcgcaatggcatcaaagaaaagcatggatttgattatgcaaaggactacttcacagacttaatcactaacgagagcattaattacttcaaaatgtctaagagaatgtatccccataggcccgttatgatggtgatcagccacgctgcgccccacggccccgaggactcagccccacagttttctaaactgtaccccaatgcttcccaacacataactcctagttataactatgcaccaaatatggataaacactggattatgcagtacacaggaccaatgctgcccatccacatggaatttacaaacattctacagcgcaaaaggctccagactttgatgtcagtggatgattctgtggagaggctgtataacatgctcgtggagacgggggagctggagaatacttacatcatttacaccgccgaccatggttaccatattgggcagtttggactggtcaaggggaaatccatgccatatgactttgatattcgtgtgcctttttttattcgtggtccaagtgtagaaccaggatcaatagtcccacagatcgttctcaacattgacttggcccccacgatcctggatattgctgggctcgacacacctcctgatgtggacggcaagtctgtcctcaaacttctggacccagaaaagccaggtaacaggtttcgaacaaacaagaaggccaaaatttggcgtgatacattcctagtggaaagaggcaaatttctacgtaagaaggaagaatccagcaagaatatccaacagtcaaatcacttgcccaaatatgaacgggtcaaagaactatgccagcaggccaggtaccagacagcctgtgaacaaccggggcagaagtggcaatgcattgaggatacatctggcaagcttcgaattcacaagtgtaaaggacccagtgacctgctcacagtccggcagagcacgcggaacctctacgctcgcggcttccatgacaaagacaaagagtgcagttgtagggagtctggttaccgtgccagcagaagccaaagaaagagtcaacggcaattcttgagaaaccaggggactccaaagtacaagcccagatttgtccatactcggcagacacgttccttgtccgtcgaatttgaaggtgaaatatatgacataaatctggaagaagaagaagaattgcaagtgttgcaaccaagaaacattgctaagcgtcatgatgaaggccacaaggggccaagagatctccaggcttccagtggtggcaacaggggcaggatgctggcagatagcagcaacgccgtgggcccacctaccactgtccgagtgacacacaagtgttttattcttcccaatgactctatccattgtgagagagaactgtaccaatcggccagagcgtggaaggaccataaggcatacattgacaaagagattgaagctctgcaagataaaattaagaatttaagagaagtgagaggacatctgaagagaaggaagcctgaggaatgtagctgcagtaaacaaagctattacaataaagagaaaggtgtaaaaaagcaagagaaattaaagagccatcttcacccattcaaggaggctgctcaggaagtagatagcaaactgcaacttttcaaggagaacaaccgtaggaggaagaaggagaggaaggagaagagacggcagaggaagggggaagagtgcagcctgcctggcctcacttgcttcacgcatgacaacaaccactggcagacagccccgttctggaacctgggatctttctgtgcttgcacgagttctaacaataacacctactggtgtttgcgtacagttaatgagacgcataattttcttttctgtgagtttgctactggctttttggagtattttgatatgaatacagatccttatcagctcacaaatacagtgcacacggtagaacgaggcattttgaatcagctacacgtacaactaatggagctcagaagctgtcaaggatataagcagtgcaacccaagacctaagaatcttgatgttggaaataaagatggaggaagctatgacctacacagaggacagttatgggatggatgggaaggttaatcagccccgtctcactgcagacatcaactggcaaggcctagaggagctacacagtgtgaatgaaaacatctatgagtacagacaaaactacagacttagtctggtggactggactaattacttgaaggatttagatagagtatttgcactgctgaagagtcactatgagcaaaataaaacaaataagactcaaactgctcaaagtgacgggttcttggttgtctctgctgagcacgctgtgtcaatggagatggcctctgctgactcagatgaagacccaaggcataaggttgggaaaacacctcatttgaccttgccagctgaccttcaaaccctgcatttgaaccgaccaacattaagtccagagagtaaacttgaatggaataacgacattccagaagttaatcatttgaattctgaacactggagaaaaaccgaaaaatggacggggcatgaagagactaatcatctggaaaccgatttcagtggcgatggcatgacagagctagagctcgggcccagccccaggctgcagcccattcgcaggcacccgaaagaacttccccagtatggtggtcctggaaaggacatttttgaagatcaactatatcttcctgtgcattccgatggaatttcagttcatcagatgttcaccatggccaccgcagaacaccgaagtaattccagcatagcggggaagatgttgaccaaggtggagaagaatcacgaaaaggagaagtcacagcacctagaaggcagcgcctcctcttcactctcctctgattagatgaaactgttaccttaccctaaacacagtatttctttttaacttttttatttgtaaactaataaaggtaatcacagccaccaacattccaagctaccctgggtacctttgtgcagtagaagctagtgagcatgtgagcaagcggtgtgcacacggagactcatcgttataatttactatctgccaagagtagaaagaaaggctggggatatttgggttggcttggttttgattttttgcttgtttgtttgttttgtactaaaacagtattatcttttgaatatcgtagggacataagtatatacatgttatccaatcaagatggctagaatggtgcctttctgagtgtctaaaacttgacacccctggtaaatctttcaacacacttccactgcctgcgtaatgaagttttgattcatttttaaccactggaatttttcaatgccgtcattttcagttagatgattttgcactttgagattaaaatgccatgtctatttgattagtcttatttttttatttttacaggcttatcagtctcactgttggctgtcattgtgacaaagtcaaataaacccccaaggacgacacacagtatggatcacatattgtttgacattaagcttttgccagaaaatgttgcatgtgttttacctcgacttgctaaaatcgattagcagaaaggcatggctaataatgttggtggtgaaaataaataaataagtaaacaaaatgaagattgcctgctctctctgtgcctagcctcaaagcgttcatcatacatcatacctttaagattgctatattttgggttattttcttgacaggagaaaaagatctaaagatcttttattttcatcttttttggttttcttggcatgactaagaagcttaaatgttgataaaatatgactagttttgaatttacaccaagaacttctcaataaaagaaaatcatgaatgctccacaatttcaacataccacaagagaagttaatttcttaacattgtgttctatgattatttgtaagaccttcaccaagttctgatatcttttaaagacatagttcaaaattgcttttgaaaatctgtattcttgaaaatatccttgttgtgtattaggtttttaaataccagctaaaggattacctcactgagtcatcagtaccctcctattcagctccccaagatgatgtgtttttgcttaccctaagagaggttttcttcttatttttagataattcaagtgcttagataaattatgttttctttaagtgtttatggtaaactcttttaaagaaaatttaatatgttatagctgaatctttttggtaactttaaatctttatcatagactctgtacatatgttcaaattagctgcttgcctgatgtgtgtatcatcggtgggatgacagaacaaacatatttatgatcatgaataatgtgctttgtaaaaagatttcaagttattaggaagcatactctgttttttaatcatgtataatattccatgatacttttatagaacaattctggcttcaggaaagtctagaagcaatatttcttcaaataaaaggtgtttaaacttt

SEQ ID NO: 8 - protein, 871 aa, Homo sapiens
MKYSCCALVLAVLGTELLGSLCSTVRSPRFRGRIQQERKNIRPNIILVLTDDQDVELGSLQVMNKTRKIMEHGGATFINAFVTTPMCCPSRSSMLTGKYVHNHNVYTNNENCSSPSWQAMHEPRTFAVYLNNTGYRTAFFGKYLNEYNGSYIPPGWREWLGLIKNSRFYNYTVCRNGIKEKHGFDYAKDYFTDLITNESINYFKMSKRMYPHRPVMMVISHAAPHGPEDSAPQFSKLYPNASQHITPSYNYAPNMDKHWIMQYTGPMLPIHMEFTNILQRKRLQTLMSVDDSVERLYNMLVETGELENTYIIYTADHGYHIGQFGLVKGKSMPYDFDIRVPFFIRGPSVEPGSIVPQIVLNIDLAPTILDIAGLDTPPDVDGKSVLKLLDPEKPGNRFRTNKKAKIWRDTFLVERGKFLRKKEESSKNIQQSNHLPKYERVKELCQQARYQTACEQPGQKWQCIEDTSGKLRIHKCKGPSDLLTVRQSTRNLYARGFHDKDKECSCRESGYRASRSQRKSQRQFLRNQGTPKYKPRFVHTRQTRSLSVEFEGEIYDINLEEEEELQVLQPRNIAKRHDEGHKGPRDLQASSGGNRGRMLADSSNAVGPPTTVRVTHKCFILPNDSIHCERELYQSARAWKDHKAYIDKEIEALQDKIKNLREVRGHLKRRKPEECSCSKQSYYNKEKGVKKQEKLKSHLHPFKEAAQEVDSKLQLFKENNRRRKKERKEKRRQRKGEECSLPGLTCFTHDNNHWQTAPFWNLGSFCACTSSNNNTYWCLRTVNETHNFLFCEFATGFLEYFDMNTDPYQLTNTVHTVERGILNQLHVQLMELRSCQGYKQCNPRPKNLDVGNKDGGSYDLHRGQLWDGWEG

SEQ ID NO: 9 - DNA, 3909 bp, Homo sapiens
  source 1..3909: mol_type = genomic DNA
gagcgagagtgtgtcgagtgagtgtgcgtctgtgtgtcccggcgagggtgcgcgctcggcgccgggagcgcggccagccgagtccggaggcatcgggaggtcgagagccgccgggaccccagctctgcgttcactgccccgtccggagctggacttcggggccggggccggggccgtgcgccggggacaggcagggccgggtcgcgggccgcgcgtcccccaggccggagatctgcgagtgaagagggacgagggaaaagaaacaaagccacagacgcaacttgagactcccgcatcccaaaagaagcaccagatcagcaaaaaaagaagatgggccccccgagcctcgtgctgtgcttgctgtccgcaactgtgttctccctgctgggtggaagctcggccttcctgtcgcaccaccgcctgaaaggcaggtttcagagggaccgcaggaacatccgccccaacatcatcctggtgctgacggacgaccaggatgtggagctgggttccatgcaggtgatgaacaagacccggcgcatcatggagcagggcggggcgcacttcatcaacgccttcgtgaccacacccatgtgctgcccctcacgctcctccatcctcactggcaagtacgtccacaaccacaacacctacaccaacaatgagaactgctcctcgccctcctggcaggcacagcacgagagccgcacctttgccgtgtacctcaatagcactggctaccggacagctttcttcgggaagtatcttaatgaatacaacggctcctacgtgccacccggctggaaggagtgggtcggactccttaaaaactcccgcttttataactacacgctgtgtcggaacggggtgaaagagaagcacggctccgactactccaaggattacctcacagacctcatcaccaatgacagcgtgagcttcttccgcacgtccaagaagatgtacccgcacaggccagtcctcatggtcatcagccatgcagccccccacggccctgaggattcagccccacaatattcacgcctcttcccaaacgcatctcagcacatcacgccgagctacaactacgcgcccaacccggacaaacactggatcatgcgctacacggggcccatgaagcccatccacatggaattcaccaacatgctccagcggaagcgcttgcagaccctcatgtcggtggacgactccatggagacgatttacaacatgctggttgagacgggcgagctggacaacacgtacatcgtatacaccgccgaccacggttaccacatcggccagtttggcctggtgaaagggaaatccatgccatatgagtttgacatcagggtcccgttctacgtgaggggccccaacgtggaagccggctgtctgaatccccacatcgtcctcaacattgacctggcccccaccatcctggacattgcaggcctggacatacctgcggatatggacgggaaatccatcctcaagctgctggacacggagcggccggtgaatcggtttcacttgaaaaagaagatgagggtctggcgggactccttcttggtggagagaggcaagctgctacacaagagagacaatgacaaggtggacgcccaggaggagaactttctgcccaagtaccagcgtgtgaaggacctgtgtcagcgtgctgagtaccagacggcgtgtgagcagctgggacagaagtggcagtgtgtggaggacgccacggggaagctgaagctgcataagtgcaagggccccatgcggctgggcggcagcagagccctctccaacctcgtgcccaagtactacgggcagggcagcgaggcctgcacctgtgacagcggggactacaagctcagcctggccggacgccggaaaaaactcttcaagaagaagtacaaggccagctatgtccgcagtcgctccatccgctcagtggccatcgaggtggacggcagggtgtaccacgtaggcctgggtgatgccgcccagccccgaaacctcaccaagcggcactggccaggggcccctgaggaccaagatgacaaggatggtggggacttcagtggcactggaggccttcccgactactcagccgccaaccccattaaagtgacacatcggtgctacatcctagagaacgacacagtccagtgtgacctggacctgtacaagtccctgcaggcctggaaagaccacaagctgcacatcgaccacgagattgaaaccctgcagaacaaaattaagaacctgagggaagtccgaggtcacctgaagaaaaagcggccagaagaatgtgactgtcacaaaatcagctaccacacccagcacaaaggccgcctcaagcacagaggctccagtctgcatcctttcaggaagggcctgcaagagaaggacaaggtgtggctgttgcgggagcagaagcgcaagaagaaactccgcaagctgctcaagcgcctgcagaacaacgacacgtgcagcatgccaggcctcacgtgcttcacccacgacaaccagcactggcagacggcgcctttctggacactggggcctttctgtgcctgcaccagcgccaacaataacacgtactggtgcatgaggaccatcaatgagactcacaatttcctcttctgtgaatttgcaactggcttcctagagtactttgatctcaacacagacccctaccagctgatgaatgcagtgaacacactggacagggatgtcctcaaccagctacacgtacagctcatggagctgaggagctgcaagggttacaagcagtgtaacccccggactcgaaacatggacctgggacttaaagatggaggaagctatgagcaatacaggcagtttcagcgtcgaaagtggccagaaatgaagagaccttcttccaaatcactgggacaactgtgggaaggctgggaaggttaagaaacaacagaggtggacctccaaaaacatagaggcatcacctgactgcacaggcaatgaaaaaccatgtgggtgatttccagcagacctgtggtattggccaggaggcctgagaaagcaagcacgcactctcagtcaacatgacagattctggaggataaccagcaggagcagagataacttcaggaagtccatttttgcccctgcttttgctttggattatacctcaccagctgcacaaaatgcattttttcgtatcaaaaagtcaccactaaccctcccccagaagctcacaaaggaaaacggagagagcgagcgagagagatttccttggaaatttctcccaagggcgaaagtcattggaatttttaaatcataggggaaaagcagtcctgttctaaatcctcttattcttttggtttgtcacaaagaaggaactaagaagcaggacagaggcaacgtggagaggctgaaaacagtgcagagacgtttgacaatgagtcagtagcacaaaagagatgacatttacctagcactataaaccctggttgcctctgaagaaactgccttcattgtatatatgtgactatttacatgtaatcaacatgggaacttttaggggaacctaataagaaatcccaattttcaggagtggtggtgtcaataaacgctctgtggccagtgtaaaagaaaatccctcgcagttgtggacatttctgttcctgtccagataccatttctcctagtatttctttgttatgtcccagaactgatgttttttttttaaggtactgaaaagaaatgaagttgatgtatgtcccaagttttgatgaaactgtatttgtaaaaaaaattttgtagtttaagtattgtcatacagtgttcaaaaccccagccaatgaccagcagttggtatgaagaacctttgacattttgtaaaaggccatttcttgggaaaaaaaaaaa

SEQ ID NO: 10 - protein, 870 aa, Homo sapiens
MGPPSLVLCLLSATVFSLLGGSSAFLSHHRLKGRFQRDRRNIRPNIILVLTDDQDVELGSMQVMNKTRRIMEQGGAHFINAFVTTPMCCPSRSSILTGKYVHNHNTYTNNENCSSPSWQAQHESRTFAVYLNSTGYRTAFFGKYLNEYNGSYVPPGWKEWVGLLKNSRFYNYTLCRNGVKEKHGSDYSKDYLTDLITNDSVSFFRTSKKMYPHRPVLMVISHAAPHGPEDSAPQYSRLFPNASQHITPSYNYAPNPDKHWIMRYTGPMKPIHMEFTNMLQRKRLQTLMSVDDSMETIYNMLVETGELDNTYIVYTADHGYHIGQFGLVKGKSMPYEFDIRVPFYVRGPNVEAGCLNPHIVLNIDLAPTILDIAGLDIPADMDGKSILKLLDTERPVNRFHLKKKMRVWRDSFLVERGKLLHKRDNDKVDAQEENFLPKYQRVKDLCQRAEYQTACEQLGQKWQCVEDATGKLKLHKCKGPMRLGGSRALSNLVPKYYGQGSEACTCDSGDYKLSLAGRRKKLFKKKYKASYVRSRSIRSVAIEVDGRVYHVGLGDAAQPRNLTKRHWPGAPEDQDDKDGGDFSGTGGLPDYSAANPIKVTHRCYILENDTVQCDLDLYKSLQAWKDHKLHIDHEIETLQNKIKNLREVRGHLKKKRPEECDCHKISYHTQHKGRLKHRGSSLHPFRKGLQEKDKVWLLREQKRKKKLRKLLKRLQNNDTCSMPGLTCFTHDNQHWQTAPFWTLGPFCACTSANNNTYWCMRTINETHNFLFCEFATGFLEYFDLNTDPYQLMNAVNTLDRDVLNQLHVQLMELRSCKGYKQCNPRTRNMDLGLKDGGSYEQYRQFQRRKWPEMKRPSSKSLGQLWEGWEG

SEQ ID NO: 11 - DNA, 5876 bp, Homo sapiens
  source 1..5876: mol_type = genomic DNA
agaacccgccccggaggggagggacgcagggaagagtcgcacggacgcactcgcgctgcggccagcgcccgggcctgcgggcccgggcggcggctgtgttgcgcagtcttcatgggttcccgacgaggaggtctctgtggctgcggcggcggctgctaactgcgccacctgctgcagcctgtccccgccgctctgaagcggccgcgtcgaagccgaaatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttgagttttgccaaccatggatggcaaatgtgatgtgctcccttccagctggtgagaggaggagttagagctggtcgttttgtgattacccataatattggaagcagcctgagggctagttaatccaaacatgcatcaacaatttggcctgagaatatgtaacagccaaaccttttcgtttagtctttattaaaatttataattggtaattggaccagttttttttttaatttccctctttttaaaacagttacggcttatttactgaataaatacaaagcaaacaaactcaagttatgtcatacctttggatacgaagaccatacataataaccaaacataacattatacacaaagaatactttcattatttgtggaatttagtgcatttcaaaaagtaatcatatatcaaactaggcaccacactaagttcctgattattttgtttataatttaataatatatcttatgagccctatatattcaaaatattatgttaacatgtaatccatgtttctttttcaaatctaaagttaaaaaaaaatagcagaagccagtgtcttaaagtctatcttttgtttctaagaccatgggatttcataatctcaagataaaatatgtatgaagtaattaatgtagaatttttacaccaaataataaataatgcttaataaactagagatatgagatgtgtaggaaatttggttaaacttttttcagatactttctggcccaaataataatttgttagcaaataatatgacccttgaactcaatggccatctattaaaagactgttgttcacactggaaaacatttaaagatgtgactatatccatgggtggattgaatcactcaaaatatattagtatccttctttagggatggttggttacagacatgtatttattcaggaggcagaaaatattccattttaattgcttattaaagaaaacattaaattctaaattattttgaggactgtgaagacttttcattagtgtaatattaggtcattgtcaatctcccagaatgtagttctatattctctaaatatgaaagtatccagaaaggccagtggtagtaaaaagcttagtgtatataatctcaaaagggatggaatatttacaactcatatttataacatgttgaatcttctcagttatcagtagtcatcagaagtgtcaatagctttctaaataaatattaaatatctactgtcctgtagtgaaggagtaatttttagtaattttctctttacaaagtctccagtgtttccaggtaaatatttgtgaaacaaaatacagcaaactacattgttacttcagtgtattgttgccaaaaatgacaagatattatattaaaatcagtaaattttagacagattttaaaaattaattagcctacaatagaggttatatggtaacacggtgatcttctaagcagttaagtgactgactgttctggcaacaacgacttctccgtgactgaagggccctgttcatttcctgatcctgaagctcgtctctcttttgagcctccgcttgctttggtcgatggtttccctcagctttttctttgctgttcttcatcctcgttgttgctgtcatcatgttcactgtggcttttacaatacagcctgtaaattccttatgacatagttcagtgcatttggctttattgcctgctccacagttctttacctttacttggcttagagaaactgtatctttgttgcttcatataacctttccccaaccccactaagctggacataacttattagtggtcctcccgtcactttatttgtagaaatctctctttcacatgagcaggggttctttcatgtggtttagctgacagcagaactagtgattctagacattttgcatggccctcattcagtggctcacaaacatgagggagcatcagaactacttgaggggcttgttaaaacccagtgcgttagaagtcggatgcggtggctcacacctgtaatcccagcactttgggaggcccaggcaggcggatcacttgaggttaggagttcaagaccagcctggccaacatggtgaaaccccgtctctactaaaaatacaaaagttagccgggtgtggtggtgcatgcctgtaatcccagcttcttgggaggccaaggcacaagaatcgcttgaaccaggagacggaggtttcagtgaatgaagatcgtgccattgtattccagcctcggcaacacagcaggactgtgattttctttggagactcctagattttctgtggttttgaactgaatttgttggatgttggcaagtgcctcttatgagctgtttctttatcctgcatttgccccacaaagacttatctggaggtgagcaaagtatgtttggtagtgaggtcacaaaggcaatcagccccttcctccccactcccattgccatcttctcagtccttctccctttctttccaagtagtttacccacccctcctctttcctcccctgtccctaaaataatccacgtgtcttcctaaaatctctctttgatcctgtcctttgataacaccgtcagtgcctactactgggtctagacagacctctgttgagcagtcagagtcttccctgactccacaatgcccctttccttggctgaccagtatgactactggtccccacctttcccttgcctatccctacctccctcctactaggttgtcccatccctctcttcacccattcattcatgaccatttttcactaccaagctccccccctcccgaaggaggctgaggtttttgtgactctctagactctattgtgggatggaatgaacattgctaaagaatcttgtgttcgctttactttaaaaaggtatttttttcctaattataaaactgatgtgtcagttacggaaaaattagaaatgcagcacaaatacatgaatattttaccacaaaattgccatataatatcttgtcttttttgggggtgtgaattttttgcattgttctggtcatattctttatcatgtaatttatgttcttttttactaagtattatgtgtggttattatagattttcacaaagatatattgctggtaatatattttattgtgtagtcttataatttacttaaccttctttcaattgttagaaatttaggctatttccagattttcagtattgtaaataatgctgtgatgaccaattttgtgaataaaatgtttttatgtatttcagattattcccttaggatagtctctcagtgccaagttgtcaaaaacatctctattttgcttatcttcctgctctcttgctgccttagggggtagtaaactgaaacataaagtaaacatgcatacaaataaaaaacataaaacaaaaataagcaacctgatggtaataggtgaaagtggtaacctgttttaactttgaattcttgccgggcgcggtggctcacgcctgtaatcccagcactttgggaggctgaggcgggtggatcacgaggtcaggagttcaaaaccagcctggccaagatggtgaaatcccgtctctactaaaaatacaaaaattagccgggcgtggtggcgggcgcctgtaatcccagctacttgggaggctgaggcagagaattgcttgaacccaggaggcggaggttgcagtgagccaagatcgcgccactgcactccagcctgggtgacagagcgagactccgtctcaaataaaaaacaacaaaaaacaaaaaaaacttaaaattctttgcttgttagtgaccttgatcatggttctctttgtacgatagttgggcatctgtatttccacttgtgtgaatttgcctttaaattttggttatgggtttcaccttttaaaataatcaaacatatttatcttttcctgtgtgataggtttttttctgtatcttttcctgttaaacacacagacccctccccaatctggacattgaataaatattcattttcctttgcattgttaaaaaaaaaaaaaaaaaaaaaaaa

SEQ ID NO: 12 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 13 - DNA, 4325 bp, Homo sapiens
  source 1..4325: mol_type = genomic DNA
cacaggtcacggggcggggccgaggcggaagcgcccgcagcccggtaccggctcctcctgggctccctctagcgccttccccccggcccgactccgctggtcagcgccaagtgacttacgcccccgaccctgagcccggaccgctaggcgaggaggatcagatctccgctcgagaatctgaaggtgccctggtcctggaggagttccgtcccagcccgcggtctcccggtactgtcgggccccggccctctggagcttcaggaggcggccgtcagggtcggggagtatttgggtccggggtctcagggaagggcggcgcctgggtctgcggtatcggaaagagcctgctggagccaagtagccctccctctcttgggacagacccctcggtcccatgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctgagggcccctcggctggcctgggcatgtgatggctcctcactgggagcctgtgggggaggctcaggtgtctggagggggtttgtgcctgataacgtaataacaccagtggagacttgcagatgtgacaattcgtccaatcctggggtaatgctgtgtgctggtgccggtcccctgtggtacgaatgaggaaactgaggtgcagagaggttcaggacttgtacaagatcacccagccagaaagaggttgggctgggatttgaaccctggtgtcgtggctctggaagctgccctggcgccttggtgatctgcgtgggtcagtgcacacaggcacacgtcagcctcaaggacatgggcacatctgttcacaggagcagcgccacgtgcctttgagtgccaggaacggggtgggagggtgggagggtgtgagggccagaagactcagaagatgcaaagtgcctgagagagacgggatattcccccagaagaagcattcttagagacacaggcactggacctccttggttcttataagaaacctgtctgaagctgggtgatgagttgcacactccaggtggggctaaggggcctggagcccctgctggctcctaggaaggcacagcagcaggccctgagacggctcctctggggcccctccaccctcccaggcctctgcatttcacctgtgcccacacttctgtctcctgccttcaccttttgacccactactaacgattctccacccagcagacaaagtgatctcttaaaaatatctgttggctgggcacggtggctcacgcctgtaatcccagcactttaggaagccgaggcgggtggatcacctgaggtcgggagttcgagaccagcctgaccaacatggagaaaccccatctctactaaaaatacaaaattagccaggtgtagtggtgcatccctgtaatcccagctacttgggagtctgaggctggagaatcacttgaacctgggaggcggtggttgcagtgagccgagatcgcaccattgcactccagcctgggcaacaagagaaaaactctgtctcaaaaaacaaaaaatctgttaggctgcacacggcgattcactcctgtattcccagtgctttgggaggctgaggtgagaggatgcctgaggccaggaattcagaccagcctgggcaacatagtgagaccccagctctaaagatttgtttttgttttttttttttttttttttttttttttttttttttttttgagacggagtctcgctctgtcgcccaggctagagtgcagtggtaccatctccgctcactgcaacctccgcctcccgggttccagggattctcctgcctcagcctccctagtagctggaactacaggtgtgtgctgccatgcccagctaatttttttttatttaatagagacaagatttcaccatgttggccaggctggtctcaaactcctgacctcaggtgatccacccgcctcagcctcccaaagtgctgggattacaggtgtgaaccaccacacctggccaacaatatttgttttaattagccaggcgtggtagcatttgtcctagcaatttgggaggttgaggtgggagaatcacttcagcccactaggtcgaggctgtagtgagctataattgtaccactgcactccagcctcggggacagagtgagaccctgtctgcaaataaacaaataaaacatcaggctgggcttgagcatctattcctgctcaaaatttcgcaggcttctcagaagaaaatccaaaccccttacagtgacccagtttgcccttgaggcctccacccacacccccttccccccagtcttagggggtggcctggctgttcccttcaacggcaacgctctgcctccattgttggcctcctctgcagggagggactgtctgagcacctgcccgtgtctgtgcagcatggcacactgacgtcaggcccacgtgcatgcccaggtggccagtcacacgccaggtgctccctcagtgttggccaagtgagaggagcacaccttccgggcgttcagacacctccccgtggcagacaccgttcgttgctaccaaacagccacctccttcctaatgggctcccatttttcagtgctgggcaaaggtcccttgatcttggagttgcagcctctttctctccaaggagggcggtgaccagcctgagccagtcaatccagtgattggttcaggagtagcctgtgaccaggagtcctggtagtgaacgactggggcagccctgggggtgaggaccttgcgcagccgtcacaggccctgattggacactgggcagctgctaacccagtgtctccagctgcctacctggagagctccaagcgtaagaaaataaaccctgcctgttgaagccaaaaaaaaaaa

SEQ ID NO: 14 - protein, 509 aa, Homo sapiens
MSMGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 15 - DNA, 6076 bp, Homo sapiens
  source 1..6076: mol_type = genomic DNA
aaaagtgaatacatgattttatttaactcattaataaggaaattggtaaggtgttaaaaccaattcaaaggacaatccaaagaacagatcaggaatactaaaataaatatgcaagcggaggtgaaactgttttccttggtagtggtggaggggaaggattgctactccgctggataaagttcatttgtgtatatataaataagaattattttccattgttatttatctataacttataaagttgtaaacaacttccacggaatcagactcaacctggaagggtatggtctctaggcaatgcaaaaattttcccctacacctgttaacaactataatatctccagacagagtagacagaaagtctggatggcaacgggaatctactggtcatacggctaacttcctaattcaataagcacgtgactaaaggattttttccttccactcagatatttcaggctaactagatactgtgtgcttcttagtgtcactgcttagtgggggagccagctctgagtggggtcatatccggacaagcgaatgagctatttattcaatgaccacgcaacactccaaatcctcccagggcaacttgaaagtaaccgcaccttccaaagggcaccgtgcaatcagactgtgtgtttggcctcctgtttgctagtggggaggaagcggcttcatgggtgtacactacgcataaatgaatgtgaaaggctatttagacctctgccttttcaccgtcctcccacctgccacaggctgggctcttgtgctagaaatgacttgctagctagacatcatggttcaggatctgagtcagaggtttaaccatttataagcttttttcttatgaaaaattggcactaattataatgtctaactgtcagagttgttgcaggctttacaggagacgcgggctgtgaagatgctttgtaaattgtgaagcgttattaaagaacacatcttttttttttaggaaaccacagtgcaaatttaattgccggggaagataacgggccttggtgccctccaagcgtcagctgagtttccaagaagccgggcagcgggcgcccgcgggttcgtctctggctcctcctccgccacagcagccgggggcccgggtcggaggcggcgggggccgagcgcccggcctcgcaagcccacggcccgctgggggtgccgtcccgcgccggggcggagcaggccccggcagcccagttcctcattctatcagcggtacaaggggctggtggcgccacaggcgctgggaccgcgggcggacaaggatgggtccgcgcggcgcggcgagcttgccccgaggccccggacctcggcggctgctcctccccgtcgtcctcccgctgctgctgctgctgttgttggcgccgccgggctcgggcgccggggccagccggccgccccacctggtcttcttgctggcagacgacctaggctggaacgacgtcggcttccacggctcccgcatccgcacgccgcacctggacgcgctggcggccggcggggtgctcctggacaactactacacgcagccgctgtgcacgccgtcgcggagccagctgctcactggccgctaccagatccgtacaggtttacagcaccaaataatctggccctgtcagcccagctgtgttcctctggatgaaaaactcctgccccagctcctaaaagaagcaggttatactacccatatggtcggaaaatggcacctgggaatgtaccggaaagaatgccttccaacccgccgaggatttgatacctactttggatatctcctgggtagtgaagattattattcccatgaacgctgtacattaattgacgctctgaatgtcacacgatgtgctcttgattttcgagatggcgaagaagttgcaacaggatataaaaatatgtattcaacaaacatattcaccaaaagggctatagccctcataactaaccatccaccagagaagcctctgtttctctaccttgctctccagtctgtgcatgagccccttcaggtccctgaggaatacttgaagccatatgactttatccaagacaagaacaggcatcactatgcaggaatggtgtcccttatggatgaagcagtaggaaatgtcactgcagctttaaaaagcagtgggctctggaacaacacggtgttcatcttttctacagataacggagggcagactttggcagggggtaataactggccccttcgaggaagaaaatggagcctgtgggaaggaggcgtccgaggggtgggctttgtggcaagccccttgctgaagcagaagggcgtgaagaaccgggagctcatccacatctctgactggctgccaacactcgtgaagctggccaggggacacaccaatggcacaaagcctctggatggcttcgacgtgtggaaaaccatcagtgaaggaagcccatcccccagaattgagctgctgcataatattgacccgaacttcgtggactcttcaccgtgtcccaggaacagcatggctccagcaaaggatgactcttctcttccagaatattcagcctttaacacatctgtccatgctgcaattagacatggaaattggaaactcctcacgggctacccaggctgtggttactggttccctccaccgtctcaatacaatgtttctgagataccctcatcagacccaccaaccaagaccctctggctctttgatattgatcgggaccctgaagaaagacatgacctgtccagagaatatcctcacatcgtcacaaagctcctgtcccgcctacagttctaccataaacactcagtccccgtgtacttccctgcacaggacccccgctgtgatcccaaggccactggggtgtggggcccttggatgtaggatttcagggaggctagaaaacctttcaattggaagttggacctcaggccttttctcacgactcttgtctcatttgttatcccaacctgggttcacttggcccttctcttgctcttaaaccacaccgaggtgtctaatttcaacccctaatgcatttaagaagctgataaaatctgcaacactcctgctgttggctggagcatgtgtctagaggtgggggtggctgggtttatccccctttcctaagccttgggacagctgggaacttaacttgaaataggaagttctcactgaatcctggaggctggaacagctggctcttttagactcacaagtcagacgttcgattcccctctgccaatagccagttttattggagtgaatcacatttcttacgcaaatgaagggagcagacagtgattaatggttctgttggccaaggcttctccctgtcggtgaaggatcatgttcaggcactccaagtgaaccacccctcttggttcaccccttactcacttatctcatcacagagcataaggcccattttgttgttcaggtcaacagcaaaatgcctgcaccatgactgtggcttttaaaataaagaaatgtgtttttatcgtaatttatttccccccagccattgctcactctgtctagacttcctgccacttccaattcttctgtggcttttcctgcctttccttttgacctcagtagtcctatccctgggaaggccactttgcttctctacctgagcacccctgatttctggaacgctgctgagccctgccttacttttgcccctagggctgaagctagaggcctccccgtaataggcggtggagttgctctgtgaggatgttcatggtagacactaagagggctgggtgggagatgcttggctctgtggcatctgttcagcgaggcttttcctatattgcatggagttagtcattgtgattgtagctttatttcataatatattaagacttgcactgctatttactagcagtgagaagaaacctcaggaaaggatatgaaaaagcaagtggccagtgtctgggatactgggccttggtaaagcagaggagggcacacccacagtcctcttattctctgttttactgcttgttttgaggttctggggtctggcaaagaggatgcagtttgacacctgcagccctttctcaatcccactaatgtcttactaatgtggaacagtccatattagctccagagagtgtcaaacccagagaaatgtgtgcaaaaatgatactcttttctgcattagccccaccattgtgttcaccaatgcttggaacactgcctgaaggcactcattttttaatttttattttatttttaattttttatatctttatgagacgatctcactctgtcaccaggttggagtacagtggtacaatcacaactcaccgtagcctcaaactcctgggctcaagtgattctcccacctcaggcacccaaatagctggaactacaggcatataccgccacacccagctaattttattttttgaaaagacaaggttccctatgttgcccagctggtcttaaactcctgggctccagcaattatcccagcttgggctccaaaagtgctgggattacaggcatgagtcaccatgcctggcctcattttttaaaacaaatgaataaatggacaaatgagtaaatgagaaagtctcacaccatgaaagatgctagtccaatgagctgaatacagaggtaatataaatgtcttccagctgttgcttttctgttctcaagctgcccctcctggggtaggagcataatctacatcactgggcagtcacaggacactctatagcaaggttgtagcgtcctctccagtggggggagaaaaggaactgtgcctaccaaaggtactctcttgtcagcaatttccatttctatactttatgggacactagaaactaaaagcaacaaataatctgatataagtccttgtatagtcatccttcaattcagtagcaatattttctggtcactactaacctgtattgtattaaaatgagactattggaaggaaatggtgctaaaactaataacatctcttaccaacctttacccaactcctgggttggcaaacagctgaccaaactgccatcacctcccacttggaagtgtatggccgacagcatgaaatagctgagcccagatgttccttctgcatcctccgaatcccagggctgggtgtaggtagccgttggaggccatcgctacagggcacctatctgttatcgctgctgtcctcccaacagctgtctccagttctagttccttggttttcaggcacagtgggggatgttctgcacccagtggacttcaaaagagttttgaagacttaattttttgtaaaacaagtacttgagattttggtttatccataatagaatgtatttcattagattctctgattctatataagaatgtgaaaagattgatatattgttgttagaaataatgttatttctttccaattttttttttttttttttttgagatggagtctcgctctgtcacccaggctggagtgcagtggtgtgatctcggctcactgcagcctctaactcccaggttcaagctattctcctgcctcagcctcccaagtagctggattacaggcatacaccaccacgcctggctatgttttgtatttttcgtagagatagggtttcaccatgttggccaggctggtctcaaactcctgacctcaagtgatccacccacttcagcttcccaaagcactgggattacaggtgtgagccactgtgcccggcaaatttttttacctttacagaaggttttgcttatttaattgtgagctcatttttctttgttacttttgtccccccagatttgggggacaaaataaaattaatcttttaaaatgtgtcagccatatgtatggggcttccatttggggtgaggagaaagttctggaactagatagtggtcatggttatacaacatcataaatgcaattactgccactgaattgtatgttttaaagtggttaaaatgttaagttttatgttttattacaatttttaaatgtgtcaaccaactttatagtacataaattatatctcagtaaagctgttaaataaataaatatagtaaaaattttagaactaaaaaaa

SEQ ID NO: 16 - protein, 533 aa, Homo sapiens
MGPRGAASLPRGPGPRRLLLPVVLPLLLLLLLAPPGSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 17 - DNA, 6377 bp, Homo sapiens
  source 1..6377: mol_type = genomic DNA
tccgcctcacattatctgcccaagcacagtgctgttggccaagcctccagcagctgacgggacccagctgtagtgaggttgcagtgattgagtaggattggcctgcttcaaagcagaggtttctcatgggaatatgcttattaaactcccactggtgcagaaaccatgaacagaggatgaacaagtgaagttgcaatctcctccatcacagctcagttccccaacaacaggatcacaagctggagatgcctttaaggaagatgaagatccctttcctcctactgttctttctgtgggaagccgagagccacgcagcatcaaggccgaacatcatcctggtgatggctgacgacctcggcattggagatcctgggtgctatgggaacaaaactatcaggactcccaatatcgaccggttggccagtgggggagtgaaactcactcagcacctggcagcatcaccgctgtgcacaccaagcagggcagccttcatgactggccggtaccctgtccgatcaggaatggcatcttggtcccgcactggagttttcctcttcacagcctcttcgggaggacttcccaccgatgagattacctttgctaagcttctgaaggatcaaggttattcaacagcactgatagggaaatggcaccttgggatgagctgtcacagcaagactgacttctgtcaccaccctttacatcacggcttcaattatttctatgggatctctttgaccaatctgagagactgcaagcccggagagggcagtgtcttcaccacgggcttcaagaggctggtcttcctccccctgcagatcgtcggggtcaccctccttacccttgctgcactcaattgtctggggctactccacgtgcctctaggcgtttttttcagccttctcttcctagcagccctaatcctgacccttttcttgggcttccttcattacttccggcccctgaactgcttcatgatgaggaactacgagatcattcagcagcccatgtcctatgacaatctcacccagaggctaacggtggaggcggcccagttcatacagcggaacactgagactccgttcctgcttgtcttgtcctacctccacgtgcacacagccctgttctccagcaaagactttgctggcaaaagtcaacacggagtctacggggatgctgttgaggaaatggactggagtgtggggcagatcttgaaccttctggatgagctgagattggctaatgataccctcatctacttcacatcggaccagggagcacatgtagaagaagtgtcttccaaaggagaaattcatggcggaagtaatgggatctataaaggaggaaaagcaaacaactgggaaggaggtatccgggttccaggcatccttcgttggcccagggtgatacaggctggccagaagattgatgagcccactagcaacatggacatatttcctacagtagccaagctggctggagctcccttgcctgaggacaggatcattgatggacgtgatctgatgcccctgcttgaaggaaaaagccaacgctccgatcatgagtttctcttccattactgcaacgcctacttaaatgctgtgcgctggcaccctcagaacagcacatccatctggaaggcctttttcttcacccccaacttcaaccccgtgggttccaacggatgctttgccacacacgtgtgcttctgtttcgggagttatgtcacccatcacgacccacctttactctttgatatttccaaagatcccagagagagaaacccactaactccagcatccgagccccggttttatgaaatcctcaaagtcatgcaggaagctgcggacagacacacccagaccctgccagaggtgcccgatcagttttcatggaacaactttctttggaagccctggcttcagctgtgctgtccttccaccggcctgtcttgccagtgtgatagagaaaaacaggataagagactgagccgctagcagcgcctggggaccagacagacgcatgtggcaaagctcaccatcttcactacaaacacgcctgagagtggcactggggaaacataactccatctacaccttggatttggactgattctccattttatcacctgaaggcttgggccagagctcaacagctactcaactggaggggtgagggggataaggtctgtagtatacagacaggaagatggtaggtttatgccttctgtggccagagtcttggactcatggaaatagaatgaatagaggggcattcacaaggcacaccagtgcaagcagatgacaaaaaggtgcagaaggcaatcttaaaacagaaaggtgcaggaggtaccttaactcacccctcagcaaatacctatgtcaacagtataagttaccatttactctataatctgcagtgatgcaataaccagcataataaaaaggcaatcacataaaaaagagtttagtcgtctaaacataagtaactttaaggtgaatgaaagatcttctttaggaataatagatgatggtaagttccactttggttattggaaggcaagtcattattactggtattagttaaaacacatatcaaatgcttgctcttcatcatatatatagttatgcatacatacacacacacacatacagtatattctttcctcaaaagggttaagatgtctaaaatagggacctagaagcttaacactatttaagtaaatacagtagaagctcacaaatagatttctttgcacaatgattttttgcaaaattttacagtaataataatcccaaggcaaatctctcctgaactgctttccattccataatttgtagtataattcttggattccactgttttctttggggaatggaagttctgaattaaaagcccactgtggagatgctgtggttcatggaatctcttccagtgtaattcagaatcattggcctagaaagtctctgatatttggaggggaacaaaaatcactcacaagcaatccatgatctatacacataagcataatttcctttagttctagttagtcatcagagaacagtcatgtatgcaagttttgtgactgagaaatttctgtgcttccaatccacaatgagatgcatgattttgttttcatcccatttcccccaagcccctgtaaatcagggaaaatgcgcaactgatcgcctaggagagggcctcgtagtggcacagctggagatagtttcaaagtctaaaccaccagcccatcctgaggaaagcctcctatggaatgtaaagtgcaatcatttcttcagatataagactttccccaacaatgtgattggattcctttatggcaaaatcgagagaagctgccatccacctgcttatgcatttatctcttttgtggacttgtctgaccaccttctatttgcccagagtttgctcaattccaagacagtgcccatgaatgggacacctgtaatgtaacccacacagcggtttgcagagaatgttagccatgacttgggctttctgaaagttggctataatttctctatccctacccacaaccctgggaagttggagcaagaggggcatactattgggctgggaggatttgacagcatttccccagttgccctttaagttcttctatttcaaacgttaattttgcttctctttctaaaaaaaaaaaaaaaaaagaaagagaagaaagaagtgattcctaccccctacctccagagttgttgaaagctgaaaagcatacaagattcttccttttaacttggatttctcgttccagaaattgtgggataatctgtattcttgctttagaaaacattcttagagagggtactagcttactgatgatgtgttaggattgctactgatgctgtcatgtggaaactatttaaaggcactattataaatttatcctataagatgacaatgtttactcaaagtctaacatattcaatgcaagtaagactttctgaaaacacttgatgatgtggaaatgctgcaggattaaataacttgaagagcctttatagattatatgaatgcctatttgtgtctagaaccagttatttaacctgtaaaatgtcaatagcaaatgaaggatgaagtatatctctagatgcaaatacattgagtttaaaagtgcctcaaaataattgagatcacatttcaggacatttggaaatcaggtcgatttgtggtaactgtagtcatcttaaatttcaaaccatttaccatctgaaagttttgatttgaatgtaaaacaggaaattggaattcctttgtccaggagaaacctcacaaaccttctttaaggcatagttttgttgtttgtttgcttgtttgttgcaggctgtaaggcatggctgcttgtttacaaagcatctcattcatattacctgtggagttgcatatccaaaccttagtgagttttgaagctttaagcaaattcttttaaaaaattcttgtatttctagcattactagatattaaaagttaagcaaatagattaatgacgtatacataggcatcatttcacaaggtcagtaatgctgcaggaaaagcaaaattgcaatctacgtatctatggtactaaggaagtcctgtttttcaaaaatggaagcccacttctcagatttttctgaagggcatacaatgaaaagtgaaggggaaacacacacacacaaaaaaacaagtatttggcttgtcacaggaatctgattgcattaagtgaaaggattatttagaatatgttaatgcaaagctaaaataaaattttccttggcaattaaaaatgctgtgcgctaataccctgctttctatcgtgactcaattcaacaatgtggggaatgtttactacatttccaacttgatgtcaagcaatggggaatacaagttccagttctgcaaagattcgtcaactttcttagctcaagagagaggctgagaaatgcagagaagaataagacataaaatagctccgacctccatgatccgagagtgggaaaaggcccgattattacccataaggcacactctctaaggccttttaaggggcctacaaaaatgttttattttataatcagaagaaaaggaaatgaacattggggattgaaaatcatattggtatttgcaccaacatagtcataaaatagtatgttaatatgtttttactttatatatttatatattaaaatatatttaatatgttttgcctttgtggcccatgaaagtcttactgggccctggggaaggtatcctaccctggtgaagcagctgctttgctctacaaatacctggggcagaaatttgatttgaaaagtattattctctcttctctttgtttcaactggattcctttggaaaaccaaactagtatcagaacaaaccccgaaacagtaagaaattggagtgagaagggcatggtattgggactaggatcggctctcattcgatcgagctattctcttaaaatgacaaaaagtgtccataaagaggctgctggagagtgcgtggccatagggagccgacatgcccgggaggaaaggtgttgattacatggatacttctaaaagctaaagccttgttgccttctctttaatgcctagagaatgggatgtgtgatgcaaatgctcaaaacctcttaaatcatagctgtctgacctctacggacctcacatccatctgaggcttcatggacaaagattctccacttggccaaactttagccaagctcctcaaccttctcccaggcccaatctgggcacttccttgtaaaatctagttttggcaagaagtctattaggtcagtttagcaagaacacctaacccccccatatctgttcaacctcagtatctgatcaggctcctcagcctccaccatcccccaggtgatgtctggtcaactggcctgccttcagctagaatcctgttaggtcggtttagatgaatgctccctgatatttcctcttggtaatcttccatccactgcccctgaccctgttccctgtctataaatccccagttttccatggtgtattcagagctgagtccagtctctctcccctactacaagtccccattgctgtggtccccgtacctgtcatgatggtcctaaataaagtcttactgtgctttaataggtagcattgaaaaatttttttctttgacatcattcatgacaacatgaaacctattgggacagcatgactgtgcagggtctttagagctcagctttctgaggccctgagcattcttggttttccgacatcgtgaacctgttctgtgttgcaagatatcactcaagccagtgttgcttaataccatctctttgtgtaatagatctgaataaagtaattgtaatacaccaa

SEQ ID NO: 18 - protein, 583 aa, Homo sapiens
MPLRKMKIPFLLLFFLWEAESHAASRPNIILVMADDLGIGDPGCYGNKTIRTPNIDRLASGGVKLTQHLAASPLCTPSRAAFMTGRYPVRSGMASWSRTGVFLFTASSGGLPTDEITFAKLLKDQGYSTALIGKWHLGMSCHSKTDFCHHPLHHGFNYFYGISLTNLRDCKPGEGSVFTTGFKRLVFLPLQIVGVTLLTLAALNCLGLLHVPLGVFFSLLFLAALILTLFLGFLHYFRPLNCFMMRNYEIIQQPMSYDNLTQRLTVEAAQFIQRNTETPFLLVLSYLHVHTALFSSKDFAGKSQHGVYGDAVEEMDWSVGQILNLLDELRLANDTLIYFTSDQGAHVEEVSSKGEIHGGSNGIYKGGKANNWEGGIRVPGILRWPRVIQAGQKIDEPTSNMDIFPTVAKLAGAPLPEDRIIDGRDLMPLLEGKSQRSDHEFLFHYCNAYLNAVRWHPQNSTSIWKAFFFTPNFNPVGSNGCFATHVCFCFGSYVTHHDPPLLFDISKDPRERNPLTPASEPRFYEILKVMQEAADRHTQTLPEVPDQFSWNNFLWKPWLQLCCPSTGLSCQCDREKQDKRLSR

SEQ ID NO: 19 - DNA, 5199 bp, Homo sapiens
  source 1..5199: mol_type = genomic DNA
cccagatcccgcagctgagagggcggaagccttggcactagcggcgcccgggcgcggagtgcgcagggcaaggtcctgcgctctgggccagcgctcggccatgcgatccgccgcgcggaggggacgcgccgcgcccgccgccagggactctttgccggtgctactgtttttatgcttgcttctgaagacgtgtgaacctaaaactgcaaatgcctttaaaccaaatatcctactgatcatggcggatgatctaggcactggggatctcggttgctacgggaacaatacactgagaacgccgaatattgaccagcttgcagaggaaggtgtgaggctcactcagcacctggcggccgccccgctctgcaccccaagccgagctgcattcctcacagggagacattccttcagatcaggcatggacgccagcaatggataccgggcccttcagtggaacgcaggctcaggtggactccctgagaacgaaaccacttttgcaagaatcttgcagcagcatggctatgcaaccggcctcataggaaaatggcaccagggtgtgaattgtgcatcccgcggggatcactgccaccaccccctgaaccacggatttgactatttctacggcatgcccttcacgctcacaaacgactgtgacccaggcaggccccccgaagtggacgccgccctgagggcgcagctctggggttacacccagttcctggcgctggggattctcaccctggctgccggccagacctgcggtttcttctctgtctccgcgagagcagtcaccggcatggccggcgtgggctgcctgtttttcatctcttggtactcctccttcgggtttgtgcgacgctggaactgtatcctgatgagaaaccatgacgtcacggagcaacccatggttctggagaaaacagcgagtcttatgctaaaggaagctgtttcctatattgaaagacacaagcatgggccatttctcctcttcctttctttgctgcatgtgcacattccccttgtgaccacgagtgcattcctggggaaaagtcagcatggcttatatggtgataatgtggaggagatggactggctcataggtaaggttcttaatgccatcgaagacaatggtttaaagaactcaacattcacgtatttcacctctgaccatggaggacatttagaggcaagagatggacacagccagttagggggatggaacggaatttacaaaggtgggaagggcatgggaggatgggaaggtgggatccgcgtgcccgggatcttccactggccgggggtgctcccggccggccgagtgattggagagcccacgagcctgatggacgtgttccctactgtggtccagctggtgggtggcgaggtgccccaggacagggtgattgatggccacagcctggtacccttgctgcagggagctgaggcacgctcggcacatgagttcctgtttcattactgtgggcagcatcttcacgcagcacgctggcaccagaaggacagtggaagcgtctggaaggttcattacacgaccccgcagttccaccccgagggagcgggggcctgctacggccgaggcgtctgcccatgctccggggagggcgtgacccatcacagaccccctttgctctttgacctctccagggacccctccgaggcacggcccctgacccccgactccgagcccctgtaccacgccgtgatagcaagggtaggtgccgcggtgtcggagcatcggcagaccctgagtcctgtgccccagcagttttccatgagcaacatcctgtggaagccgtggctgcagccgtgctgcggacatttcccgttctgttcatgccacgaggatggggatggcaccccctgaatgccaggactgtgagagaggatccaggagagcctgactgcgttgcaaacaaaattctccaagcttggttctatcttcagcttccctttttgcaaggaacatgccctggactgagagtgggtccccactttctttctttctttctttcttttttgagacagagtgtcgctctgtccctcaggctggaatgcaatggcacgatctctgctcactgcaacctccgcctcccgggttcaagcgattttcctgcctcagcctcctgagtacccaggattacaggcaccaggcacctgccaccatgcctagctaatttttgtagttttagtagagacagggtttcaccatgttgcccaggctggtctcgcactcctgacctcaagtcatccacctgcctcagcctcccaaagtgctgggattacaggcacgagccactgcgcccatgtagggtttccctttcctgatttgtgaaataagactgtcccagtaggcacccactgatgcctcctcttcctcttctaaatctcagggttcgtcattgtgccaatgcccgatgttttcacccctccgtcttaaagcattgttgcaatttcatcacctagatgacataacagccttacaaaaggacagggaggagtgtctgttcctactctcacatagcggaggaaagttagagcctctcagtctctgtttatgaggactcattaatctcaaataattgatgcatttttcatacattagggtctctgtccatgtgtcttcctgatattgttatagaaatggcttcaggctgctggtaacagatgctgcggaaaaagaatgccttaaacaaagccaggcacggtgactcacacctgtaatcccagcactttgggaggctaaggtgggaggatcacttgagcctaggagttagacacctacccagcctgagcaacacagtgagacctcatctctacaaaaaacaaataattagctagatgtcgtggcgcacagctgtagtctcagctacttaggaggctgaggcgggaggattatttgagcctgggaggtcaaaactgcaatgagctaagattgcactactgcactccgggctgggagacagagtaagaccctgccttaaaaaaaaaaaaaaaaaaatgccttaaagaaaataataagagaagggtgtgttctcttccaagtaatgagccgatcaaggggaagcaacccaaggctagcaggctcttttcaattcccagctctgccactcttagcatggcagagagttggcctcattatgccagtatggctgccacagtgtcagacatcacattccaggcagtggaaaggagaaagaaccaaagggcatccctttctcttgaagcagtccctgtgaggggggattatctggaagccccaacccaatttctgcttccaaccattatccatagaaacaactggaagttgagaagtgcagtcttttaggtgggcacctggctgccctgtatgaaaccagaattaggttggtcagcaggaaaacaagaaaacacttactgtgttgactgaggtttttgacgcatcgctttattgtcaaattaaacaactctatctcattctgcactgaaacacgtactaccattgcctataattggaaaatgatcctcagaggcacagaagatgccctgatggaaagtttgccccttgggcaaagagacagccatgggaaccttcagacatagagagagaaggtggcttttctccctacattcctcaaatagctaagatttggccagtgtgttttctaagtcaattctagtgtgtttcatcctcacctcttccctctgtggctttgatgatggaagtgtagtcctaacctactgcacagctgggaccccctgcccctagatcccaatactggggatgggaggaccttgcactattcccctcagtccatctatcgaggtctttgcaggaagcatactgggaattgaaacgagagcctaaatgacatctaagaaaggcagtgttcaataccaggtattaggtgaggatgggattctaaggacatcagtgggaggcagggagccaccttcagacctcagcatggaagcttccaagatccagaggaagaggcaacagcactgagagtcataggtagaagaatcatcacagccctgctaaccaggcagctgatgcccctctcccctggctccctgtgtccaaatcctacaggggcatctgttggctgaactcaacctgaagccaaagagaagatgagtggagagaggcaacatttatagagctcaggtttctagggctggagagggatctggagggacacacaggagacacctggcataaccaaaaaatgattaaaaaaaaaaaaaaaaagaaacatctatggagcatgggacacggggagtggaggcagctaaaagccatctcatctttcaccgtattcaacaagcccattataacaagtctttcacgctcaaccattcaaacaatcccaacaatcccagcctaagaatttcctggcatttgatgaaaatggctgtgggttttgctatctttaagctccctgggaagcaggatataagcccagggctggcaggctctttctagctacctgctcttgcacatagagtttgcctcatggttgcaagaaggctgccatagctccagatggcacatagacattccaggcagcaggaaaaaggaaggagcacttaaccgaaggaggtcagggagttggttagtctccacctgaagaagagagccatgaaccagcttcagttgactaacgggctcctgtgagtgcatctttggacttttctggaggttgaaatctagatgtggtatgtgtcttaaagcagccaccaactcctcccattaccttccaagtgagcccaactacacgatggagcctcttccctgcccttggatctgggctgagcctctgacttgcgttgaccaacagaatgcagtgaaagtgatgccgatactaccctccctgccctagacttgggatacctggcagctatattcttccattcctcaggacttgcaaaaacgggtctcagcatgccttcccagagccatgaggtgtttctttgcccattcattgatctgagaagtgagtgtaaggagtttaaatcaacctctgttctgtgctagttaaggtaataaagttgctgcagtccagggggtaggtacctgtggacggctctgcgaataggacagttgcatttcttgggaatcaagtgcatccctaggctggcagtgcagcagaaatactgaataaaatgtgacaaatctccctggaaaaaaaaaaaaaaaaa

SEQ ID NO: 20 - protein, 593 aa, Homo sapiens
MRSAARRGRAAPAARDSLPVLLFLCLLLKTCEPKTANAFKPNILLIMADDLGTGDLGCYGNNTLRTPNIDQLAEEGVRLTQHLAAAPLCTPSRAAFLTGRHSFRSGMDASNGYRALQWNAGSGGLPENETTFARILQQHGYATGLIGKWHQGVNCASRGDHCHHPLNHGFDYFYGMPFTLTNDCDPGRPPEVDAALRAQLWGYTQFLALGILTLAAGQTCGFFSVSARAVTGMAGVGCLFFISWYSSFGFVRRWNCILMRNHDVTEQPMVLEKTASLMLKEAVSYIERHKHGPFLLFLSLLHVHIPLVTTSAFLGKSQHGLYGDNVEEMDWLIGKVLNAIEDNGLKNSTFTYFTSDHGGHLEARDGHSQLGGWNGIYKGGKGMGGWEGGIRVPGIFHWPGVLPAGRVIGEPTSLMDVFPTVVQLVGGEVPQDRVIDGHSLVPLLQGAEARSAHEFLFHYCGQHLHAARWHQKDSGSVWKVHYTTPQFHPEGAGACYGRGVCPCSGEGVTHHRPPLLFDLSRDPSEARPLTPDSEPLYHAVIARVGAAVSEHRQTLSPVPQQFSMSNILWKPWLQPCCGHFPFCSCHEDGDGTP

SEQ ID NO: 21 - DNA, 2220 bp, Homo sapiens
  source 1..2220: mol_type = genomic DNA
ttggtagacgtgggcgtggctttgagattcccccatgctgcgatgtggggggagtctgctctgtctgtcctaactctctctgatcttctgacttgggaaaaacaaactcgaagttaatcattcccagctcaaagccttgtgcaagtgctctctgccttcacgcttgcttcctttgggagagaaccttcctcttcttgatcggggattcaggaaggagcccaggagcagaggaagtagagagagagacaacatgttacatctgcaccattcttgtttgtgtttcaggagctggctgccagcgatgctcgctgtactgctaagtttggcaccatcagcttccagcgacatttccgcctcccgaccgaacatccttcttctgatggcggacgaccttggcattggggacattggctgctatggcaacaacaccatgaggactccgaatattgaccgccttgcagaggacggcgtgaagctgacccaacacatctctgccgcatctttgtgcaccccaagcagagccgccttcctcacgggcagataccctgtgcgatcagggatggtttccagcattggttaccgtgttcttcagtggaccggagcatctggaggtcttccaacaaatgagacaacttttgcaaaaatactgaaagagaaaggctatgccactggactcattggaaaatggcatctgggtctcaactgtgagtcagccagtgatcattgccaccaccctctccatcatggctttgaccatttctacggaatgcctttctccttgatgggtgattgcgcccgctgggaactctcagagaagcgtgtcaacctggaacaaaaactcaacttcctcttccaagtcctggccttggttgccctcacactggtagcagggaagctcacacacctgatacccgtctcgtggatgccggtcatctggtcagccctttcggccgtcctcctcctcgcaagctcctattttgtgggtgctctgattgtccatgccgattgctttctgatgagaaaccacaccatcacggagcagcccatgtgcttccaaagaacgacaccccttattctgcaggaggttgcgtcctttctcaaaaggaataagcatgggcctttcctcctctttgtttcctttctacacgttcacatccctcttatcactatggagaacttcctcgggaagagtctccacgggctgtatggggacaacgtagaggagatggactggatggtaggacggatccttgacactttggacgtggagggtttgagcaacagcaccctcatttattttacgtcggatcacggcggttccctagagaatcaacttggaaacacccagtatggtggctggaatggaatttataaaggtgggaagggcatgggaggatgggaaggtgggatccgcgtgcccgggatcttccgctggcccggggtgctcccggccggccgagtgattggcgagcccacgagtctgatggacgtgttccccaccgtggtccggctggcgggcggcgaggtgccccaggacagagtgattgacggccaagaccttctgcccttgctcctggggacagcccaacactcagaccacgagttcctgatgcattattgtgagaggtttctgcacgcagccaggtggcatcaacgggacagaggaacaatgtggaaagtccactttgtgacgcctgtgttccagccagagggagccggtgcctgctatggaagaaaggtctgcccgtgctttggggaaaaagtagtccaccacgatccacctttgctctttgacctctcaagagacccttctgagacccacatcctcacaccagcctcagagcccgtgttctatcaggtgatggaacgagtccagcaggcggtgtgggaacaccagcggacactcagcccagttcctctgcagctggacaggctgggcaacatctggagaccgtggctgcagccctgctgtggcccgttccccctctgctggtgccttagggaagatgacccacaataaatgtctgcagtgaaaagctggagccccgattcctaaattttgtcactcaaattgaaacaaaccagctggccatggtggttgtcatcccagcactttaggaggccaccacaggaggatcactcccgtgatcaaaaccaacctgggcaacatgatgaaactctagctctacaaaacaaaaataaaaaaaaaattagcctgca

SEQ ID NO: 22 - protein, 589 aa, Homo sapiens
MLHLHHSCLCFRSWLPAMLAVLLSLAPSASSDISASRPNILLLMADDLGIGDIGCYGNNTMRTPNIDRLAEDGVKLTQHISAASLCTPSRAAFLTGRYPVRSGMVSSIGYRVLQWTGASGGLPTNETTFAKILKEKGYATGLIGKWHLGLNCESASDHCHHPLHHGFDHFYGMPFSLMGDCARWELSEKRVNLEQKLNFLFQVLALVALTLVAGKLTHLIPVSWMPVIWSALSAVLLLASSYFVGALIVHADCFLMRNHTITEQPMCFQRTTPLILQEVASFLKRNKHGPFLLFVSFLHVHIPLITMENFLGKSLHGLYGDNVEEMDWMVGRILDTLDVEGLSNSTLIYFTSDHGGSLENQLGNTQYGGWNGIYKGGKGMGGWEGGIRVPGIFRWPGVLPAGRVIGEPTSLMDVFPTVVRLAGGEVPQDRVIDGQDLLPLLLGTAQHSDHEFLMHYCERFLHAARWHQRDRGTMWKVHFVTPVFQPEGAGACYGRKVCPCFGEKVVHHDPPLLFDLSRDPSETHILTPASEPVFYQVMERVQQAVWEHQRTLSPVPLQLDRLGNIWRPWLQPCCGPFPLCWCLREDDPQ

SEQ ID NO: 23 - DNA, 2048 bp, Homo sapiens
  source 1..2048: mol_type = genomic DNA
aaaagaggcagagcctttgcagaacagcctgtctgttctgctcctagacattagagagataatacggctgatagacaacaagaaggtattccaagctgcacaatgaggcccaggagacccttggtcttcatgtctttggtgtgtgcactcttgaacacatgccaggcacacagggtgcatgacgacaagcctaatattgtcctaatcatggttgatgacctgggtattggagatctgggctgctacggcaatgacaccatgaggacgcctcacatcgaccgccttgccagggaaggcgtgcgactgactcagcacatctctgccgcctccctctgcagcccaagccggtccgcgttcttgacgggaagataccccatccgatcaggtatggtttctagtggtaatagacgtgtcatccaaaatcttgcagtccccgcaggcctccctcttaatgagacaacacttgcagccttgctaaagaagcaaggatacagcacggggcttataggcaaatggcaccaaggcttgaactgcgactcccgaagtgaccagtgccaccatccatataattatgggtttgactactactatggcatgccgttcactctcgttgacagctgctggccggacccctctcgtaacacggaattagcctttgagagtcagctctggctctgtgtgcagctagttgccattgccatcctcaccctaacctttgggaagctgagcggctgggtctctgttccctggctcctgatcttctccatgattctgtttattttcctcttgggctatgcttggttctccagccacacgtcccctttatactgggactgcctcctcatgcgggggcacgagatcacggagcagcccatgaaggctgaacgagctggatccattatggtgaaggaagcgatttcctttttagaaaggcacagtaaggaaactttccttctctttttctcctttcttcacgtgcacacacctctccccaccacggacgatttcactggcaccagcaagcatggcttgtatggggataatgtggaagagatggactccatggtgggcaagattcttgatgctatcgatgattttggcctaaggaacaacacccttgtctactttacatcagatcacggagggcatttggaagctaggcgagggcatgcccaacttggtggatggaatggaatatacaaaggtggaaaaggcatggggggctgggaaggtggaatccgcgtcccaggaattgtccgatggcctggaaaggtaccagctggacggttgattaaggaacctacaagtttaatggatattttaccaactgtcgcatcagtgtcaggaggaagtctccctcaggacagggtcattgacggccgagacctcatgcccttgctgcagggcaacgtcaggcactcggagcatgaatttcttttccactactgtggctcctacctgcacgccgtgcggtggatccccaaggacgacagtgggtcagtttggaaggctcactatgtgaccccggtattccagccaccagcttctggtggctgctatgtcacctcattatgcagatgtttcggagaacaggttacctaccacaacccccctctgctcttcgatctctccagggacccctcagagtccacacccctgacacctgccacagagcccctccatgattttgtgattaaaaaggtggccaacgccctgaaggaacaccaggaaaccatcgtgcctgtgacctaccaactctcagaactgaatcagggcaggacgtggctgaagccttgctgtggggtgttcccattttgtctgtgtgacaaggaagaggaagtctctcagcctcggggtcctaacgagaagagataattacaatcaggctaccagaggaagcctttggtcctaacgagaagagataattacaatcaggctaccaaaggaagcactaactttggtgctttcaagttggcaaggagtgcatttaatagtcaataaattcatctaccattccagattattaaaggcccactggttgttccact

SEQ ID NO: 24 - protein, 590 aa, Homo sapiens
MRPRRPLVFMSLVCALLNTCQAHRVHDDKPNIVLIMVDDLGIGDLGCYGNDTMRTPHIDRLAREGVRLTQHISAASLCSPSRSAFLTGRYPIRSGMVSSGNRRVIQNLAVPAGLPLNETTLAALLKKQGYSTGLIGKWHQGLNCDSRSDQCHHPYNYGFDYYYGMPFTLVDSCWPDPSRNTELAFESQLWLCVQLVAIAILTLTFGKLSGWVSVPWLLIFSMILFIFLLGYAWFSSHTSPLYWDCLLMRGHEITEQPMKAERAGSIMVKEAISFLERHSKETFLLFFSFLHVHTPLPTTDDFTGTSKHGLYGDNVEEMDSMVGKILDAIDDFGLRNNTLVYFTSDHGGHLEARRGHAQLGGWNGIYKGGKGMGGWEGGIRVPGIVRWPGKVPAGRLIKEPTSLMDILPTVASVSGGSLPQDRVIDGRDLMPLLQGNVRHSEHEFLFHYCGSYLHAVRWIPKDDSGSVWKAHYVTPVFQPPASGGCYVTSLCRCFGEQVTYHNPPLLFDLSRDPSESTPLTPATEPLHDFVIKKVANALKEHQETIVPVTYQLSELNQGRTWLKPCCGVFPFCLCDKEEEVSQPRGPNEKR

SEQ ID NO: 25 - DNA, 2785 bp, Homo sapiens
  source 1..2785: mol_type = genomic DNA
ggctgcgcccaggccggcgggcccagcagctgcgaaccgccggcgcaccacctgtttccgcgcccggggacttccccggcggggctcagaagtgtgggatcggtcgcttggcttcccctggcgtcagcgacccagggtaacctcctccactgctgcgtgccgtgcaggcctgcctgtgtgagagccacgtgtgccgcgctctgggcacagccttggaaagtcaggaccgcgacggcagcagagcagaaaccttacagaaacatgaagccctcaaccatctgctactcagttattcggggctgacggcggcttctagaacatccaggtgttctgcagatgcgagaactcatcctgtagtcaccagatggagtcccaaacagccaagcagatgtaaggcctgtgctgtggctctgaggccctgaatacagaagggtcactttcttagtggccaaagagcagttgttgacattgatgtctaattattgaacacgaccagtcattttactgagctgcggtgaggaaacactgaccatagaagatcaagccaaatgagggattgcaaatttcctgattcttttgaattaggattccagatgggggcctcatttctacagcccccaacattcctatagccgttatcactgccatcaccactgccaccagcatcttcttgcagattccacccctgctccccagagacttcctgctttgaaagtgagcagaaaggaagctctcagaaaaatctctagtggtggctgccgtcgctccagacaatcggaatcctgccttcaccaccatgggctggctttttctaaaggttttgttggcgggagtgagtttctcaggatttctttatcctcttgtggatttttgcatcagtgggaaaacaagaggacagaagccaaactttgtgattattttggccgatgacatggggtggggtgacctgggagcaaactgggcagaaacaaaggacactgccaaccttgataagatggcttcggagggaatgaggtttgtggatttccatgcagctgcctccacctgctcaccctcccgggcttccttgctcaccggccggcttggccttcgcaatggagtcacacgcaactttgcagtcacttctgtgggaggccttccgctcaacgagaccaccttggcagaggtgctgcagcaggcgggttacgtcactgggataataggcaaatggcatcttggacaccacggctcttatcaccccaacttccgtggttttgattactactttggaatcccatatagccatgatatgggctgtactgatactccaggctacaaccaccctccttgtccagcgtgtccacagggtgatggaccatcaaggaaccttcaaagagactgttacactgacgtggccctccctctttatgaaaacctcaacattgtggagcagccggtgaacttgagcagccttgcccagaagtatgctgagaaagcaacccagttcatccagcgtgcaagcaccagcgggaggcccttcctgctctatgtggctctggcccacatgcacgtgcccttacctgtgactcagctaccagcagcgccacggggcagaagcctgtatggtgcagggctctgggagatggacagtctggtgggccagatcaaggacaaagttgaccacacagtgaaggaaaacacattcctctggtttacaggagacaatggcccgtgggctcagaagtgtgagctagcgggcagtgtgggtcccttcactggattttggcaaactcgtcaagggggaagtccagccaagcagacgacctgggaaggagggcaccgggtcccagcactggcttactggcctggcagagttccagttaatgtcaccagcactgccttgttaagcgtgctggacatttttccaactgtggtagccctggcccaggccagcttacctcaaggacggcgctttgatggtgtggacgtctccgaggtgctctttggccggtcacagcctgggcacagggtgctgttccaccccaacagcggggcagctggagagtttggagccctgcagactgtccgcctggagcgttacaaggccttctacattaccggtggagccagggcgtgtgatgggagcacggggcctgagctgcagcataagtttcctctgattttcaacctggaagacgataccgcagaagctgtgcccctagaaagaggtggtgcggagtaccaggctgtgctgcccgaggtcagaaaggttcttgcagacgtcctccaagacattgccaacgacaacatctccagcgcagattacactcaggacccttcagtaactccctgctgtaatccctaccaaattgcctgccgctgtcaagccgcataacagaccaatttttattccacgaggaggagtacctggaaattaggcaagtttgcttccaaatttcatttttaccctctttacaaacacacgctttagtttagtcttggagtttagttttggagttagccttgcatatcccttctgtatcctgtccctcctccacgccgacccgagagcagctgagctgcgctggctctgggcagggagtgtgccttaatgggaagcacacgggctttggagtcaggcacaggtgccagctccagcttttgaacttgggcaattgtttaacctaacctgcaagttgattttgagggttaaataaaggcatacatgaaaatgcctggcaaattacctgacacagagcagacattcaatacattttagtttccttgtttcaaaaaaaaaaaaaaa

SEQ ID NO: 26 - protein, 525 aa, Homo sapiens
MGWLFLKVLLAGVSFSGFLYPLVDFCISGKTRGQKPNFVIILADDMGWGDLGANWAETKDTANLDKMASEGMRFVDFHAAASTCSPSRASLLTGRLGLRNGVTRNFAVTSVGGLPLNETTLAEVLQQAGYVTGIIGKWHLGHHGSYHPNFRGFDYYFGIPYSHDMGCTDTPGYNHPPCPACPQGDGPSRNLQRDCYTDVALPLYENLNIVEQPVNLSSLAQKYAEKATQFIQRASTSGRPFLLYVALAHMHVPLPVTQLPAAPRGRSLYGAGLWEMDSLVGQIKDKVDHTVKENTFLWFTGDNGPWAQKCELAGSVGPFTGFWQTRQGGSPAKQTTWEGGHRVPALAYWPGRVPVNVTSTALLSVLDIFPTVVALAQASLPQGRRFDGVDVSEVLFGRSQPGHRVLFHPNSGAAGEFGALQTVRLERYKAFYITGGARACDGSTGPELQHKFPLIFNLEDDTAEAVPLERGGAEYQAVLPEVRKVLADVLQDIANDNISSADYTQDPSVTPCCNPYQIACRCQAA

SEQ ID NO: 27 - DNA, 1689 bp, Homo sapiens
  source 1..1689: mol_type = genomic DNA
atgacaagaaacgccagacccaacattgtcctgctgatggcagatgaccttggagtgggggatttgtgctgctacggtaataactcagtgagcacacctaatattgaccgcctggcaagtgaaggagtgaggcttacccagcatctcgcagctgcttccatgtgcaccccaagtcgggctgccttcctgaccggccggtaccccatcagatcagggatggtgtctgcctacaacctgaaccgtgccttcacgtggcttggtgggtcaggtggtcttcccaccaatgaaacgacttttgccaagctgctgcagcaccgtggctaccgcacgggactcataggcaaatggcacctgggtttgagctgcgcctctcggaatgatcactgttaccacccgctcaaccatggttttcactacttttacggggtgccttttggacttttaagcgactgccaggcatccaagacaccagaactgcaccgctggctcaggatcaaactgtggatctccacggtagcccttgccctggttccttttctgcttctcattcccaagttcgcccgctggttctcagtgccatggaaggtcatctttgtctttgctctcctcgcctttctgtttttcacttcctggtactctagttatggatttactcgacgttggaattgcatccttatgaggaaccatgaaattatccagcagccaatgaaagaggagaaagtagcttccctcatgctgaaggaggcacttgctttcattgaaaggtacaaaagggaaccttttctcctctttttttccttcctgcacgtacatactccactcatctccaaaaagaagtttgttgggcgcagtaaatatggcaggtatggggacaatgtagaagaaatggattggatggtgggtaaaatcctggatgccctggaccaggagcgcctggccaaccacaccttggtgtacttcacctctgacaacgggggccacctggagcccctggacggggctgttcagctgggtggctggaacgggatctacaaaggtggcaaaggaatgggaggatgggaaggaggtatccgtgtgccagggatattccggtggccgtcagtcttggaggctgggagagtgatcaatgagcccaccagcttaatggacatctatccgacgctgtcttatataggcggagggatcttgtcccaggacagagtgattgacggccagaacctaatgcccctgctggaaggaagggcgtcccactccgaccacgagttcctcttccactactgtggggtctatctgcacacggtcaggtggcatcagaaggactgtgcaactgtgtggaaagctcattatgtgactcctaaattctaccctgaaggaacaggtgcctgctatgggagtggaatatgttcatgttcgggggatgtaacctaccacgacccaccactcctctttgacatctcaagagacccttcagaagcccttccactgaaccctgacaatgagccattatttgactccgtgatcaaaaagatggaggcagccataagagagcatcgtaggacactaacacctgtcccacagcagttctctgtgttcaacacaatttggaaaccatggctgcagccttgctgtgggaccttccccttctgtgggtgtgacaaggaagatgacatccttcccatggctccctga

SEQ ID NO: 28 - protein, 562 aa, Homo sapiens
MTRNARPNIVLLMADDLGVGDLCCYGNNSVSTPNIDRLASEGVRLTQHLAAASMCTPSRAAFLTGRYPIRSGMVSAYNLNRAFTWLGGSGGLPTNETTFAKLLQHRGYRTGLIGKWHLGLSCASRNDHCYHPLNHGFHYFYGVPFGLLSDCQASKTPELHRWLRIKLWISTVALALVPFLLLIPKFARWFSVPWKVIFVFALLAFLFFTSWYSSYGFTRRWNCILMRNHEIIQQPMKEEKVASLMLKEALAFIERYKREPFLLFFSFLHVHTPLISKKKFVGRSKYGRYGDNVEEMDWMVGKILDALDQERLANHTLVYFTSDNGGHLEPLDGAVQLGGWNGIYKGGKGMGGWEGGIRVPGIFRWPSVLEAGRVINEPTSLMDIYPTLSYIGGGILSQDRVIDGQNLMPLLEGRASHSDHEFLFHYCGVYLHTVRWHQKDCATVWKAHYVTPKFYPEGTGACYGSGICSCSGDVTYHDPPLLFDISRDPSEALPLNPDNEPLFDSVIKKMEAAIREHRRTLTPVPQQFSVFNTIWKPWLQPCCGTFPFCGCDKEDDILPMAP

SEQ ID NO: 29 - DNA, 3225 bp, Homo sapiens
  source 1..3225: mol_type = genomic DNA
gcgtcccgcccctccacctggggctcggcccggcccggcagatgttacaactttttcgaattctctcccgccgtgtcccctcgacccgcccaacttgtgcctccctccccttcccctctggggtcctgcccacctccctgcagggagctgggctgttttaaggactccgggtggggcgagaggccgggaaagcagaggagagagaaattaggaggcgggagaaatccagggcaagaaggaagaggggagtcagaggatggtagagagcactttttggaagctgccacgccgcgtctcaggctggccgggctgagctggggaagagggagcaaaggcggcgcagggcctgcgcttaggcagcgggaggcagctcggcgcgggcctgacctccccagagcgccccgctgcggccgagcagatccggcccagccgtccggcagccagtcccggaccagacactggaccgtccccggggggcgctgaactccctcgcagcatccgagccggcgggccggtggtgcgccctgggcgcgcgaggtggtgaggccccaggagcccggcgcgccgggacgcgcgggccggcttggcgatgcacaccctcactggcttctccctggtcagcctgctcagcttcggctacctgtcctgggactgggccaagccgagcttcgtggccgacgggcccggggaggctggcgagcagccctcggccgctccgccccagcctccccacatcatcttcatcctcacggacgaccaaggctaccacgacgtgggctaccatggttcagatatcgagacccctacgctggacaggctggcggccaagggggtcaagttggagaattattacatccagcccatctgcacgccttcgcggagccagctcctcactggcaggtaccagatccacacaggactccagcattccatcatccgcccacagcagcccaactgcctgcccctggaccaggtgacactgccacagaagctgcaggaggcaggttattccacccatatggtgggcaagtggcacctgggcttctaccggaaggagtgtctgcccacccgtcggggcttcgacaccttcctgggctcgctcacgggcaatgtggactattacacctatgacaactgtgatggcccaggcgtgtgcggcttcgacctgcacgagggtgagaatgtggcctgggggctcagcggccagtactccactatgctttatgcccagcgcgccagccatatcctggccagccacagccctcagcgtcccctcttcctctatgtggccttccaggcagtacacacacccctgcagtcccctcgtgagtacctgtaccgctaccgcaccatgggcaatgtggcccggcggaagtacgcggccatggtgacctgcatggatgaggctgtgcgcaacatcacctgggccctcaagcgctacggtttctacaacaacagtgtcatcatcttctccagtgacaatggtggccagactttctcggggggcagcaactggccgctccgaggacgcaagggcacttattgggaaggtggcgtgcggggcctaggctttgtccacagtcccctgctcaagcgaaagcaacggacaagccgggcactgatgcacatcactgactggtacccgaccctggtgggtctggcaggtggtaccacctcagcagccgatgggctagatggctacgacgtgtggccggccatcagcgagggccgggcctcaccacgcacggagatcctgcacaacattgacccactctacaaccatgcccagcatggctccctggagggcggctttggcatctggaacaccgccgtgcaggctgccatccgcgtgggtgagtggaagctgctgacaggagaccccggctatggcgattggatcccaccgcagacactggccaccttcccgggtagctggtggaacctggaacgaatggccagtgtccgccaggccgtgtggctcttcaacatcagtgctgacccttatgaacgggaggacctggctggccagcggcctgatgtggtccgcaccctgctggctcgcctggccgaatataaccgcacagccatcccggtacgctacccagctgagaacccccgggctcatcctgactttaatgggggtgcttgggggccctgggccagtgatgaggaagaggaggaagaggaagggagggctcgaagcttctcccggggtcgtcgcaagaaaaaatgcaagatttgcaagcttcgatcctttttccgtaaactcaacaccaggctaatgtcccaacggatctgatggtggggagggagaaaactgtcctttagaggatcttccccactccggcttggccctgctgtttctcagggagaagcctgtcacatctccatctacagggagttggagggtgtagagtcccttggttgaacagggtagggagcctggataggagtgggtgggaataaaccagactgggatgcctgtgtctcagtcctgcctcctcacggacttgctctgtgacctcaggtgacccacatgagcttttagcctcagtttcctcatctgtaaaatgagctctaatgactttgtgactctttggtgtggccctggagcctggggccacggtggagttcctggccggccttgccacttgacaactcctttaaggcttcccccttaacacgggatccctgtggtggtgtttgggagttgcctggaggcaactccaagcctggcccccagctgaagcatggcaatctggctgctctctacagggacccccaagcgctgtgggtggagggcaggggtcgggggggttgaccttcttgggtcttcacatggcctaggccagtcctccggtcagactggtgtcaggcaccgtggtgcaaaattcctcttctggcccctccagtacccagagaaactggctgggccattaactgctgcagcaccaagggtggtagaaagagctgtgaagagcccccaaaccagtaccaggacacctgggttctcctgtgacctggggcacagttcttgccctctaggccttgatttccccacctgcaagtggggatgccagccctggctctgcctccttcatgaggctctggaagactggccaaggttgtggaggagcttgtgaacttgattaaagtgtcgtaacatgaaaaaaaaaaaaaaaaaaaaaaaaaaaaaaaaaaaaaaaaaaaaaaaaaaaaaaaaaa

SEQ ID NO: 30 - protein, 569 aa, Homo sapiens
MHTLTGFSLVSLLSFGYLSWDWAKPSFVADGPGEAGEQPSAAPPQPPHIIFILTDDQGYHDVGYHGSDIETPTLDRLAAKGVKLENYYIQPICTPSRSQLLTGRYQIHTGLQHSIIRPQQPNCLPLDQVTLPQKLQEAGYSTHMVGKWHLGFYRKECLPTRRGFDTFLGSLTGNVDYYTYDNCDGPGVCGFDLHEGENVAWGLSGQYSTMLYAQRASHILASHSPQRPLFLYVAFQAVHTPLQSPREYLYRYRTMGNVARRKYAAMVTCMDEAVRNITWALKRYGFYNNSVIIFSSDNGGQTFSGGSNWPLRGRKGTYWEGGVRGLGFVHSPLLKRKQRTSRALMHITDWYPTLVGLAGGTTSAADGLDGYDVWPAISEGRASPRTEILHNIDPLYNHAQHGSLEGGFGIWNTAVQAAIRVGEWKLLTGDPGYGDWIPPQTLATFPGSWWNLERMASVRQAVWLFNISADPYEREDLAGQRPDVVRTLLARLAEYNRTAIPVRYPAENPRAHPDFNGGAWGPWASDEEEEEEEGRARSFSRGRRKKKCKICKLRSFFRKLNTRLMSQRI

SEQ ID NO: 31 - DNA, 4689 bp, Homo sapiens
  source 1..4689: mol_type = genomic DNA
ctggagagaaaagagggaggaggcaaaagaactcggagtgccaaagctaaataagttagctgagaaaacgcacgcagtttgcagcgcctgcgccgggtgcgccaactacgcaaagaccaagcgggctccgcgcggaccggccgcggggctagggacccggctttggccttcaggctccctagcagcggggaaaaggaattgctgcccggagtttctgcggaggtggagggagatcaggaaacggcttcttcctcacttcgccgcctggtgagtgtcggggagattggcaaacgcctaggaaaggactggggaaaatagccctgggaaagtggagaaggtgatcaggaggccggtccactacggcagtttatctgtctgatcagagccagacgcgacgcgtccacttcgcagttctttccaggtgtggggaccgcaggacagacggccgatcccgccgccctccgtaccagcactcccaggagagtcagcctcgctccccaacgtcgagggcgctctggccacgaaaagttcctgtccactgtgattctcaattccttgcttggtttttttctccagagaacttttgggtggagatattaacttttttcttttttttttccttggtggaagctgctctagggaggggggaggaggaggagaaagtgaaatgtgctggagaagagcgagccctccttgttcttccggagtcccatccattaagccatcacttctggaagattaaagttgtcggacatggtgacagctgagaggagaggaggatttcttgccaggtggagagtcttcaccgtctgttgggtgcatgtgtgcgcccgcagcggcgcggggcgcgtggttctccgcgtggagtctcacctgggacctgagtgaatggctcccaggggctgtgcggggcatccgcctccgccttctccacaggcctgtgtctgtcctggaaagatgctagcaatgggggcgctggcaggattctggatcctctgcctcctcacttatggttacctgtcctggggccaggccttagaagaggaggaagaaggggccttactagctcaagctggagagaaactagagcccagcacaacttccacctcccagccccatctcattttcatcctagcggatgatcagggatttagagatgtgggttaccacggatctgagattaaaacacctactcttgacaagctcgctgccgaaggagttaaactggagaactactatgtccagcctatttgcacaccatccaggagtcagtttattactggaaagtatcagatacacaccggacttcaacattctatcataagacctacccaacccaactgtttacctctggacaatgccaccctacctcagaaactgaaggaggttggatattcaacgcatatggtcggaaaatggcacttgggtttttacagaaaagaatgcatgcccaccagaagaggatttgataccttttttggttcccttttgggaagtggggattactatacacactacaaatgtgacagtcctgggatgtgtggctatgacttgtatgaaaacgacaatgctgcctgggactatgacaatggcatatactccacacagatgtacactcagagagtacagcaaatcttagcttcccataaccccacaaagcctatatttttatatattgcctatcaagctgttcattcaccactgcaagctcctggcaggtatttcgaacactaccgatccattatcaacataaacaggaggagatatgctgccatgctttcctgcttagatgaagcaatcaacaacgtgacattggctctaaagacttatggtttctataacaacagcattatcatttactcttcagataatggtggccagcctacggcaggagggagtaactggcctctcagaggtagcaaaggaacatattgggaaggagggatccgggctgtaggctttgtgcatagcccacttctgaaaaacaagggaacagtgtgtaaggaacttgtgcacatcactgactggtaccccactctcatttcactggctgaaggacagattgatgaggacattcaactagatggctatgatatctgggagaccataagtgagggtcttcgctcaccccgagtagatattttgcataacattgaccccatatacaccaaggcaaaaaatggctcctgggcagcaggctatgggatctggaacactgcaatccagtcagccatcagagtgcagcactggaaattgcttacaggaaatcctggctacagcgactgggtcccccctcagtctttcagcaacctgggaccgaaccggtggcacaatgaacggatcaccttgtcaactggcaaaagtgtatggcttttcaacatcacagccgacccatatgagagggtggacctatctaacaggtatccaggaatcgtgaagaagctcctacggaggctctcacagttcaacaaaactgcagtgccggtcaggtatccccccaaagaccccagaagtaaccctaggctcaatggaggggtctggggaccatggtataaagaggaaaccaagaaaaagaagccaagcaaaaatcaggctgagaaaaagcaaaagaaaagcaaaaaaaagaagaagaaacagcagaaagcagtctcaggttcaacttgccattcaggtgttacttgtggataagcacaaatatttcctgtttggttaaactttaatcagttcttatctttcatctgtttcctaggtaaaccagcaaatttggctcgataatatcgctggcctaagcgtcaggcttgttttcatgctgtgccactccagagacttctgccacctggccgccacactgaaaactgtcctgctcagtgccaaggtgctactcttgcaagccacacttagagagagtggagatgtttatttctcttgctcctttagaaaacgtggtgagtcctgagttccactgctgtgcttcagtcaactgaccaaacactgctttgaattataggaggagaacaataacctaccatccgcaagcatgctaatttgatggaagttacagggtataccatccgcaagcatgctaatttgatggaagttacagggtagcatgattaaaactacctttgataaattacagtcaaagattgtgtcacctcaaaggccttgaagaatatattttcttggtgaatttttgtatgtctgtcatatgacacttgggttttttaattaattctattttatatatatatatatgtttcttttcctgtgaaaagctgtttttctcacatgtgaacagcttgcacctcattttaccatgcgtgagggaatggcaaataagaatgtttgagcacactgcccacaatgaatgtaactattttctaaacactttactagaagaacatttcagtataaaaaacctaatttatttttacagaaaaatattttgttgtttttataaaaagttatgcaaatgactttattttatttttatttcctgcataccattagaataattttatttcatttcttcaaattaccaagcactgtaatactataaattaatgtaatactgtgtgaattcagactataaaaaacatcattcagaaaactttataatcgtcattgttcaaccaagattttgaatgtaataagatgaatatattccttacaaattacttggaaattcaatgtttgtgcagagttgagacaactttattgtttctatcataaactatttatgtatcttaattattaaaatgatttactttatggcactagaaaatttactgtggcttttctgatctaacttctagctaaaattgtatcattggtcctaaaaaataaaaatctttactaataggcaattgaaggaatggtttgctaacaaccacagtaatataatatgattttacagatagatgcttccccttggctatgacatggagaaagattttcccataataataactaacatttatattaggttggtgcaaaactagttgcggtttttcccattaaaagtaataaacttactcttatacaaagtggacactgtggggagatacagagaaatggaagatacggatcctgcctggagtaggtaaccttgcttggaaaccccacatgcaaacgtcatgaggagaattaaaggagtattatcagtaatgaagtttatcatgggtcatcaatgagcatagattggtgtggatcctgtagaccctggtgttttctttgaagtgccctctcctaatgcagaggccttgaagcttacagtatacacttgaaaagtcacagatagctagaattatgatctttgaagttataactgtgatctgaaaatgtgtgtggtggtatgacagcataccattaaatacatttacatcacagctcaaaggactgtgatataatccatttatatcacaactcaaaggactgtgatataatccatttatatcacagctcacagtttctgaaaatgtataaaagaatctataatctagtactgaaattactaaattgggtaagatgatttaaatgattttaattttaacattttatttctagaatatatggctccattttattttatagtgtaaagttgtatttcctaaagtttgtgttttgtcgacagtatcttttaaatgagtcttaaaaataaaggcatattgttcatgttaaaaaaaaaaa

SEQ ID NO: 32 - protein, 599 aa, Homo sapiens
MAPRGCAGHPPPPSPQACVCPGKMLAMGALAGFWILCLLTYGYLSWGQALEEEEEGALLAQAGEKLEPSTTSTSQPHLIFILADDQGFRDVGYHGSEIKTPTLDKLAAEGVKLENYYVQPICTPSRSQFITGKYQIHTGLQHSIIRPTQPNCLPLDNATLPQKLKEVGYSTHMVGKWHLGFYRKECMPTRRGFDTFFGSLLGSGDYYTHYKCDSPGMCGYDLYENDNAAWDYDNGIYSTQMYTQRVQQILASHNPTKPIFLYIAYQAVHSPLQAPGRYFEHYRSIININRRRYAAMLSCLDEAINNVTLALKTYGFYNNSIIIYSSDNGGQPTAGGSNWPLRGSKGTYWEGGIRAVGFVHSPLLKNKGTVCKELVHITDWYPTLISLAEGQIDEDIQLDGYDIWETISEGLRSPRVDILHNIDPIYTKAKNGSWAAGYGIWNTAIQSAIRVQHWKLLTGNPGYSDWVPPQSFSNLGPNRWHNERITLSTGKSVWLFNITADPYERVDLSNRYPGIVKKLLRRLSQFNKTAVPVRYPPKDPRSNPRLNGGVWGPWYKEETKKKKPSKNQAEKKQKKSKKKKKKQQKAVSGSTCHSGVTCG

SEQ ID NO: 33 - DNA, 3345 bp, Homo sapiens
  source 1..3345: mol_type = genomic DNA
aggagttgtagttctgcgggtgaagctcggcgttactatcaagcaaccaaactgcaagctttgggagttgttcgctgtccctgccctgctctgctagggagagaacgccagagggaggcggctggcccggcggcaggctctcagaaccgctaccggcgatgctactgctgtgggtgtcggtggtcgcagccttggcgctggcggtactggcccccggagcaggggagcagaggcggagagcagccaaagcgcccaatgtggtgctggtcgtgagcgactccttcgatggaaggttaacatttcatccaggaagtcaggtagtgaaacttccttttatcaactttatgaagacacgtgggacttcctttctgaatgcctacacaaactctccaatttgttgcccatcacgcgcagcaatgtggagtggcctcttcactcacttaacagaatcttggaataattttaagggtctagatccaaattatacaacatggatggatgtcatggagaggcatggctaccgaacacagaaatttgggaaactggactatacttcaggacatcactccattagtaatcgtgtggaagcgtggacaagagatgttgctttcttactcagacaagaaggcaggcccatggttaatcttatccgtaacaggactaaagtcagagtgatggaaagggattggcagaatacagacaaagcagtaaactggttaagaaaggaagcaattaattacactgaaccatttgttatttacttgggattaaatttaccacacccttacccttcaccatcttctggagaaaattttggatcttcaacatttcacacatctctttattggcttgaaaaagtgtctcatgatgccatcaaaatcccaaagtggtcacctttgtcagaaatgcaccctgtagattattactcttcttatacaaaaaactgcactggaagatttacaaaaaaagaaattaagaatattagagcattttattatgctatgtgtgctgagacagatgccatgcttggtgaaattattttggcccttcatcaattagatcttcttcagaaaactattgtcatatactcctcagaccatggagagctggccatggaacatcgacagttttataaaatgagcatgtacgaggctagtgcacatgttccgcttttgatgatgggaccaggaattaaagccggcctacaagtatcaaatgtggtttctcttgtggatatttaccctaccatgcttgatattgctggaattcctctgcctcagaacctgagtggatactctttgttgccgttatcatcagaaacatttaagaatgaacataaagtcaaaaacctgcatccaccctggattctgagtgaattccatggatgtaatgtgaatgcctccacctacatgcttcgaactaaccactggaaatatatagcctattcggatggtgcatcaatattgcctcaactctttgatctttcctcggatccagatgaattaacaaatgttgctgtaaaatttccagaaattacttattctttggatcagaagcttcattccattataaactaccctaaagtttctgcttctgtccaccagtataataaagagcagtttatcaagtggaaacaaagtataggacagaattattcaaacgttatagcaaatcttaggtggcaccaagactggcagaaggaaccaaggaagtatgaaaatgcaattgatcagtggcttaaaacccatatgaatccaagagcagtttgaacaaaaagtttaaaaatagtgttctagagatacatataaatatattacaagatcataattatgtattttaaatgaaacagttttaataattaccaagttttggccgggcacagtggctcacacctgtaatcccaggactttgggaggctgaggaaagcagatcacaaggtcaagagattgagaccatcctggccaacatggtgaaaccctgtctctactaaaaatacaaaaattagctgggcgcggtggtgcacacctatagtctcagctactcagaggctgaggcaggaggatcgcttgaacccgggaggcagcagttgcagtgagctgagattgcgccactgtactccagcctggcaacagagtgagactgtgtcgcaaaaaaataaaaataaaataataataattaccaatttttcattattttgtaagaatgtagtgtattttaagataaaatgccaatgattataaaatcacatattttcaaaaatggttattatttaggcctttgtacaatttctaacaatttagtggaagtatcaaaagaattgaagcaaatactgtaacagttatgttcctttaaataatagagaatataaaatattgtaataatatgtatcataaaatagttgtatgtgagcatttgatggtgctcgatgagttacttgtatttgatgggattgtttggatgtatttaatgggagtatttggagtatttaacgggatgtaaaccctggatgtacctgattttgttactgttttattttaataggtaatatatatacagggtaaaagcttcaaatggtacaaaagggttaacagtgatcgtgaagtctctgtcctttccctcttccctgccatccagttccccctccaagaagcaagtaccgaaaccacctgcttacgcatttttagagattggccacaaatttataaacaaatgtatatattcctttccccctacacaaacggtaacatactgcacacattgttctgcatgttgcttctttttccttttttttttcacttaacagtagatatctagaggtgaaattactgagtcaagactatatttagcaaaattacactagatactacaaattacctctaaagaaggtatactaactgataatctcaccatcaatgcatgtcttcttatcctttgccaacctaacagataaaaatgttctatttttatttttctttttatgagtaacgtagagcatattttcatgtatttaacagccactggaatctgctttaccatggcctttcctatttctattctttgcctatttttctgttggttgttggtctttgttttgtattacaggtgtgctttagatattagctttttgtaagagatcctgcaaatatcttctttccagtttgtcattgtcatttgtcttttgactttgttctggtattttttgatatgtagaaatttttattttcatgtaagcaaatttatgaatctttgtacaccataagtatatacaattatgatttgtcaattaaaaatattagtacaaaatttacagatctttgcttttgtggcttttgggattttgtatca

SEQ ID NO: 34 - protein, 536 aa, Homo sapiens
MLLLWVSVVAALALAVLAPGAGEQRRRAAKAPNVVLVVSDSFDGRLTFHPGSQVVKLPFINFMKTRGTSFLNAYTNSPICCPSRAAMWSGLFTHLTESWNNFKGLDPNYTTWMDVMERHGYRTQKFGKLDYTSGHHSISNRVEAWTRDVAFLLRQEGRPMVNLIRNRTKVRVMERDWQNTDKAVNWLRKEAINYTEPFVIYLGLNLPHPYPSPSSGENFGSSTFHTSLYWLEKVSHDAIKIPKWSPLSEMHPVDYYSSYTKNCTGRFTKKEIKNIRAFYYAMCAETDAMLGEIILALHQLDLLQKTIVIYSSDHGELAMEHRQFYKMSMYEASAHVPLLMMGPGIKAGLQVSNVVSLVDIYPTMLDIAGIPLPQNLSGYSLLPLSSETFKNEHKVKNLHPPWILSEFHGCNVNASTYMLRTNHWKYIAYSDGASILPQLFDLSSDPDELTNVAVKFPEITYSLDQKLHSIINYPKVSASVHQYNKEQFIKWKQSIGQNYSNVIANLRWHQDWQKEPRKYENAIDQWLKTHMNPRAV

SEQ ID NO: 35 - DNA, 2179 bp, Homo sapiens
  source 1..2179: mol_type = genomic DNA
cacgtgaccaaccgggtcacatggcccgcgggacaacatggctgcgcccgcactagggctggtgtgtggacgttgccctgagctgggtctcgtcctcttgctgctgctgctctcgctgctgtgtggagcggcagggagccaggaggccgggaccggtgcgggcgcggggtcccttgcgggttcttgcggctgcggcacgccccagcggcctggcgcccatggcagttcggcagccgctcaccgatactcgcgggaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccactatggactgacaaccaaggaaagtcttccccagtccaaggagcagtcgtgtctgacctacattgggcttttctcagaactttgaacgatcccatgcaaagaattcccaccctgaggtgggttacatacctgcccaatggccaaaggaaccgccttgtgagaccaaattgctgacctgggtcagtgcatgtgctttatggtgtggtgcatctttggagatcatcaccatattttacttttgagagtctttaaagaggaaggggagtggagggaaccctgagctaggcttcaggaggcccgcgtcctacgcaggctctgccacaggggttagaccccaggtccgacgcttgaccttcctgggcctcaagtgccctcccctatcaaatgaagggatggacagcatgacctctgggtgtctctccaactcaccagttctaaaaagggtatcagattctattgtgacttcatagtgagaatttattatagattattttttagctattttttccatgtgtgaaccttgagtgatactaatcatgtaaagtaagagttctcttatgtattattttcggaagaggggtgtggtgactcctttatattcgtactgcactttgtttttccaaggaaatcagtgtcttttacgttgttatgatgaatcccacatggggccggtgatggtatgctgaagttcagccgttgaacacataggaatgtctgtggggtgactctactgtgctttatcttttaacattaagtgcctttggttcagaggggcagtcataagctctgtttccccctctccccaaagccttcagcgaacgtgaaatgtgcgctaaacggggaaacctgtttaattctagatatagggaaaaaggaacgaggaccttgaatgagctatattcagggtatccggtattttgtaatagggaataggaaaccttgttggctgtggaatatccgatgctttgaatcatgcactgtgttgaataaacgtatctgctaaatcaggcaaaaaaaaaaaaaaaaaa

SEQ ID NO: 36 - protein, 374 aa, Homo sapiens
MAAPALGLVCGRCPELGLVLLLLLLSLLCGAAGSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMD

SEQ ID NO: 37 - protein, 28 aa, Solanum tuberosum
MASSATTKSFLILFFMILATTSSTCAVD

SEQ ID NO: 38 - DNA, 1524 bp, synthetic construct
  source 1..1524: mol_type = other DNA
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggaactctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgcatcatcatcatcatcat

SEQ ID NO: 39 - protein, 508 aa, synthetic construct
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELHHHHHH

SEQ ID NO: 40 - DNA, 1524 bp, synthetic construct
  source 1..1524: mol_type = other DNA
atgtcttgtccagttccagcttgttgcgctctccttcttgttcttggattgtgtagggcaaggcctaggaacgctcttttgcttcttgctgatgatggcggattcgagtccggtgcttacaacaactctgctatcgctactccacacctcgatgctcttgctaggcgttctcttcttttccgtaacgctttcacttccgtgtcctcttgctcaccttctagggcttcacttcttactggacttccacagcaccagaacggaatgtacggacttcatcaggatgtgcaccacttcaactcattcgataaggtgagatccctcccactcctcttgtctcaagctggtgttaggactggaatcatcggcaaaaagcacgtgggaccagagactgtgtacccattcgatttcgcttacactgaggaaaacggctccgttcttcaagtgggcagaaatattactaggatcaagctcctcgtgaggaagttcctccagactcaagatgataggccattcttcctctacgtggcattccatgatccacataggtgcggacattctcagccacagtacggaactttctgcgagaagttcggaaacggtgagtctggtatgggcaggattccagattggactccacaggcttacgatccacttgatgtgctcgttccatacttcgtgccaaacactccagctgctagagctgatcttgctgctcagtacactactgtgggaaggatggatcagggtgtgggacttgtgcttcaagagcttagagatgctggcgtgctcaacgatactctcgtgatcttcacttcagataacggcatcccattcccatccggaaggactaatctttactggccaggtactgctgagcctctccttgtttcttcaccagagcatccaaagaggtggggacaagtttctgaggcttacgtgtcccttctcgatctcactccaactatcctcgattggttctccatcccttacccatcctacgctatcttcggctccaagactatccaccttactggcagatctttgctcccagctttggaagctgaaccactttgggctactgtgttcggatctcagtctcaccacgaggtgacaatgtcttacccaatgagatctgtgcagcacaggcacttcaggcttgttcacaacctcaacttcaagatgccattcccaatcgatcaggatttctacgtgtcaccaactttccaggatcttctcaacaggactactgcaggacaacctactggctggtacaaggatcttaggcactactactatagggctaggtgggagctttacgataggtccagagatccacacgagactcagaaccttgctactgatccaaggttcgctcagctccttgagatgcttagggatcagcttgctaagtggcagtgggagactcatgatccatgggtttgcgctccagatggtgtgcttgaagagaagttgtctccacagtgccagccacttcataacgagcttcatcatcaccatcaccac

SEQ ID NO: 41 - protein, 508 aa, synthetic construct; identical to SEQ ID NO: 39

SEQ ID NO: 42 - DNA, 1552 bp, synthetic construct
  source 1..1552: mol_type = other DNA
aacaatggcttcctccgctactactaaatctttccttattcttttctttatgatacttgcaactacttcatcaacatgtgcagtcgaccgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggaactctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgcatcatcatcatcatcat

SEQ ID NO: 43 - protein, 516 aa, synthetic construct
MASSATTKSFLILFFMILATTSSTCAVDRPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELHHHHHH

SEQ ID NO: 44 - DNA, 1552 bp, synthetic construct
  source 1..1552: mol_type = other DNA
aacaatggcttcctccgctactactaaatctttccttattcttttctttatgatacttgcaactacttcatcaacatgtgcagtcgacaggcctaggaacgctcttttgcttcttgctgatgatggcggattcgagtccggtgcttacaacaactctgctatcgctactccacacctcgatgctcttgctaggcgttctcttcttttccgtaacgctttcacttccgtgtcctcttgctcaccttctagggcttcacttcttactggacttccacagcaccagaacggaatgtacggacttcatcaggatgtgcaccacttcaactcattcgataaggtgagatccctcccactcctcttgtctcaagctggtgttaggactggaatcatcggcaaaaagcacgtgggaccagagactgtgtacccattcgatttcgcttacactgaggaaaacggctccgttcttcaagtgggcagaaatattactaggatcaagctcctcgtgaggaagttcctccagactcaagatgataggccattcttcctctacgtggcattccatgatccacataggtgcggacattctcagccacagtacggaactttctgcgagaagttcggaaacggtgagtctggtatgggcaggattccagattggactccacaggcttacgatccacttgatgtgctcgttccatacttcgtgccaaacactccagctgctagagctgatcttgctgctcagtacactactgtgggaaggatggatcagggtgtgggacttgtgcttcaagagcttagagatgctggcgtgctcaacgatactctcgtgatcttcacttcagataacggcatcccattcccatccggaaggactaatctttactggccaggtactgctgagcctctccttgtttcttcaccagagcatccaaagaggtggggacaagtttctgaggcttacgtgtcccttctcgatctcactccaactatcctcgattggttctccatcccttacccatcctacgctatcttcggctccaagactatccaccttactggcagatctttgctcccagctttggaagctgaaccactttgggctactgtgttcggatctcagtctcaccacgaggtgacaatgtcttacccaatgagatctgtgcagcacaggcacttcaggcttgttcacaacctcaacttcaagatgccattcccaatcgatcaggatttctacgtgtcaccaactttccaggatcttctcaacaggactactgcaggacaacctactggctggtacaaggatcttaggcactactactatagggctaggtgggagctttacgataggtccagagatccacacgagactcagaaccttgctactgatccaaggttcgctcagctccttgagatgcttagggatcagcttgctaagtggcagtgggagactcatgatccatgggtttgcgctccagatggtgtgcttgaagagaagttgtctccacagtgccagccacttcataacgagcttcatcatcaccatcaccac

SEQ ID NO: 45 - protein, 516 aa, synthetic construct; identical to SEQ ID NO: 43

SEQ ID NO: 46 - DNA, 2326 bp, synthetic construct
  source 1..2326: mol_type = other DNA
aacaatggcttcctccgctactactaaatctttccttattcttttctttatgatacttgcaactacttcatcaacatgtgcagtcgaccctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtactacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattatttctcgagcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggaactctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgcatcatcatcatcatcat

SEQ ID NO: 47 - protein, 774 aa, synthetic construct
MASSATTKSFLILFFMILATTSSTCAVDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLFLERPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELHHHHHH

SEQ ID NO: 48 - DNA, 2326 bp, synthetic construct
  source 1..2326: mol_type = other DNA
aacaatggcttcctccgctactactaaatctttccttattcttttctttatgatacttgcaactacttcatcaacatgtgcagtcgaccctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtactacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattatttctcgagaggcctaggaacgctcttttgcttcttgctgatgatggcggattcgagtccggtgcttacaacaactctgctatcgctactccacacctcgatgctcttgctaggcgttctcttcttttccgtaacgctttcacttccgtgtcctcttgctcaccttctagggcttcacttcttactggacttccacagcaccagaacggaatgtacggacttcatcaggatgtgcaccacttcaactcattcgataaggtgagatccctcccactcctcttgtctcaagctggtgttaggactggaatcatcggcaaaaagcacgtgggaccagagactgtgtacccattcgatttcgcttacactgaggaaaacggctccgttcttcaagtgggcagaaatattactaggatcaagctcctcgtgaggaagttcctccagactcaagatgataggccattcttcctctacgtggcattccatgatccacataggtgcggacattctcagccacagtacggaactttctgcgagaagttcggaaacggtgagtctggtatgggcaggattccagattggactccacaggcttacgatccacttgatgtgctcgttccatacttcgtgccaaacactccagctgctagagctgatcttgctgctcagtacactactgtgggaaggatggatcagggtgtgggacttgtgcttcaagagcttagagatgctggcgtgctcaacgatactctcgtgatcttcacttcagataacggcatcccattcccatccggaaggactaatctttactggccaggtactgctgagcctctccttgtttcttcaccagagcatccaaagaggtggggacaagtttctgaggcttacgtgtcccttctcgatctcactccaactatcctcgattggttctccatcccttacccatcctacgctatcttcggctccaagactatccaccttactggcagatctttgctcccagctttggaagctgaaccactttgggctactgtgttcggatctcagtctcaccacgaggtgacaatgtcttacccaatgagatctgtgcagcacaggcacttcaggcttgttcacaacctcaacttcaagatgccattcccaatcgatcaggatttctacgtgtcaccaactttccaggatcttctcaacaggactactgcaggacaacctactggctggtacaaggatcttaggcactactactatagggctaggtgggagctttacgataggtccagagatccacacgagactcagaaccttgctactgatccaaggttcgctcagctccttgagatgcttagggatcagcttgctaagtggcagtgggagactcatgatccatgggtttgcgctccagatggtgtgcttgaagagaagttgtctccacagtgccagccacttcataacgagcttcatcatcaccatcaccac

SEQ ID NO: 49 - protein, 774 aa, synthetic construct; identical to SEQ ID NO: 47

SEQ ID NO: 50 - DNA, 2353 bp, synthetic construct
  source 1..2353: mol_type = other DNA
aacaatggcttcctccgctactactaaatctttccttattcttttctttatgatacttgcaactacttcatcaacatgtgcagtcgacggagaaacttctactcttaggacttcattcacaagaaacatcgttggtcgtgatggattgtgcgtggatgtgaggaatggatacgacactgatggaactccacttcagttgtggccatgtggaacccagagaaaccaacgatggacttttgactcagacgatacaatcaggtcaatgggtaaatgcatgactgcaaacgggttaaacaatggaagcaatatcgtgatattcaactgttctacagctgctgagaacgccattaagtgggaagtacctattgatggcagcatcatcaatccttcctccggattggttatgaccgctcctcgtgctgcatcccgtaccatcctgttgcttgaggacaatatctacgccgctagccagggttggactgtgacaaacaatgtaaagcccatcgttgcttccattgtgggttataaagaaatgtgcttgcagtctaacggtgaaaacaatggtgtttggatggaggattgtgaggccaccagtttgcagcaacagtgggcactctatggtgaccgtaccatccgagtaaatagtactcgtggcttatgcgtcaccaccaatgggtacaactccaaggatctcatcatcatccttaaatgccaaggattgcccagccagaggtggtttttcaactccgacggcgccatcgtaaacccaaagtcaagacatgttatggatgtgagagcaagcaatgtctctcttcgagaaatcattatctttccagccactgggaaccctaatcagcaatgggtgacacaagtccttccaagtctcgagcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggaactctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgcatcatcatcatcatcat

SEQ ID NO: 51 - protein, 783 aa, synthetic construct [sequence omitted]

SEQ ID NO: 52 - DNA, 2353 bp, synthetic construct
  source 1..2353: mol_type = other DNA
aacaatggcttcctccgctactactaaatctttccttattcttttctttatgatacttgcaactacttcatcaacatgtgcagtcgacggagaaacttctactcttaggacttcattcacaagaaacatcgttggtcgtgatggattgtgcgtggatgtgaggaatggatacgacactgatggaactccacttcagttgtggccatgtggaacccagagaaaccaacgatggacttttgactcagacgatacaatcaggtcaatgggtaaatgcatgactgcaaacgggttaaacaatggaagcaatatcgtgatattcaactgttctacagctgctgagaacgccattaagtgggaagtacctattgatggcagcatcatcaatccttcctccggattggttatgaccgctcctcgtgctgcatcccgtaccatcctgttgcttgaggacaatatctacgccgctagccagggttggactgtgacaaacaatgtaaagcccatcgttgcttccattgtgggttataaagaaatgtgcttgcagtctaacggtgaaaacaatggtgtttggatggaggattgtgaggccaccagtttgcagcaacagtgggcactctatggtgaccgtaccatccgagtaaatagtactcgtggcttatgcgtcaccaccaatgggtacaactccaaggatctcatcatcatccttaaatgccaaggattgcccagccagaggtggtttttcaactccgacggcgccatcgtaaacccaaagtcaagacatgttatggatgtgagagcaagcaatgtctctcttcgagaaatcattatctttccagccactgggaaccctaatcagcaatgggtgacacaagtccttccaagtctcgagaggcctaggaacgctcttttgcttcttgctgatgatggcggattcgagtccggtgcttacaacaactctgctatcgctactccacacctcgatgctcttgctaggcgttctcttcttttccgtaacgctttcacttccgtgtcctcttgctcaccttctagggcttcacttcttactggacttccacagcaccagaacggaatgtacggacttcatcaggatgtgcaccacttcaactcattcgataaggtgagatccctcccactcctcttgtctcaagctggtgttaggactggaatcatcggcaaaaagcacgtgggaccagagactgtgtacccattcgatttcgcttacactgaggaaaacggctccgttcttcaagtgggcagaaatattactaggatcaagctcctcgtgaggaagttcctccagactcaagatgataggccattcttcctctacgtggcattccatgatccacataggtgcggacattctcagccacagtacggaactttctgcgagaagttcggaaacggtgagtctggtatgggcaggattccagattggactccacaggcttacgatccacttgatgtgctcgttccatacttcgtgccaaacactccagctgctagagctgatcttgctgctcagtacactactgtgggaaggatggatcagggtgtgggacttgtgcttcaagagcttagagatgctggcgtgctcaacgatactctcgtgatcttcacttcagataacggcatcccattcccatccggaaggactaatctttactggccaggtactgctgagcctctccttgtttcttcaccagagcatccaaagaggtggggacaagtttctgaggcttacgtgtcccttctcgatctcactccaactatcctcgattggttctccatcccttacccatcctacgctatcttcggctccaagactatccaccttactggcagatctttgctcccagctttggaagctgaaccactttgggctactgtgttcggatctcagtctcaccacgaggtgacaatgtcttacccaatgagatctgtgcagcacaggcacttcaggcttgttcacaacctcaacttcaagatgccattcccaatcgatcaggatttctacgtgtcaccaactttccaggatcttctcaacaggactactgcaggacaacctactggctggtacaaggatcttaggcactactactatagggctaggtgggagctttacgataggtccagagatccacacgagactcagaaccttgctactgatccaaggttcgctcagctccttgagatgcttagggatcagcttgctaagtggcagtgggagactcatgatccatgggtttgcgctccagatggtgtgcttgaagagaagttgtctccacagtgccagccacttcataacgagcttcatcatcaccatcaccac

SEQ ID NO: 53 - protein, 783 aa, synthetic construct [sequence omitted]

SEQ ID NO: 54 - DNA, 2298 bp, synthetic construct
  source 1..2298: mol_type = other DNA
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggaactctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgctcgagcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtactacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattatttcatcatcatcaccaccac

SEQ ID NO: 55 - protein, 766 aa, synthetic construct [sequence omitted]

SEQ ID NO: 56 - DNA, 2298 bp, synthetic construct
  source 1..2298: mol_type = other DNA
atgtcttgtccagttccagcttgttgcgctctccttcttgttcttggattgtgtagggcaaggcctaggaacgctcttttgcttcttgctgatgatggcggattcgagtccggtgcttacaacaactctgctatcgctactccacacctcgatgctcttgctaggcgttctcttcttttccgtaacgctttcacttccgtgtcctcttgctcaccttctagggcttcacttcttactggacttccacagcaccagaacggaatgtacggacttcatcaggatgtgcaccacttcaactcattcgataaggtgagatccctcccactcctcttgtctcaagctggtgttaggactggaatcatcggcaaaaagcacgtgggaccagagactgtgtacccattcgatttcgcttacactgaggaaaacggctccgttcttcaagtgggcagaaatattactaggatcaagctcctcgtgaggaagttcctccagactcaagatgataggccattcttcctctacgtggcattccatgatccacataggtgcggacattctcagccacagtacggaactttctgcgagaagttcggaaacggtgagtctggtatgggcaggattccagattggactccacaggcttacgatccacttgatgtgctcgttccatacttcgtgccaaacactccagctgctagagctgatcttgctgctcagtacactactgtgggaaggatggatcagggtgtgggacttgtgcttcaagagcttagagatgctggcgtgctcaacgatactctcgtgatcttcacttcagataacggcatcccattcccatccggaaggactaatctttactggccaggtactgctgagcctctccttgtttcttcaccagagcatccaaagaggtggggacaagtttctgaggcttacgtgtcccttctcgatctcactccaactatcctcgattggttctccatcccttacccatcctacgctatcttcggctccaagactatccaccttactggcagatctttgctcccagctttggaagctgaaccactttgggctactgtgttcggatctcagtctcaccacgaggtgacaatgtcttacccaatgagatctgtgcagcacaggcacttcaggcttgttcacaacctcaacttcaagatgccattcccaatcgatcaggatttctacgtgtcaccaactttccaggatcttctcaacaggactactgcaggacaacctactggctggtacaaggatcttaggcactactactatagggctaggtgggagctttacgataggtccagagatccacacgagactcagaaccttgctactgatccaaggttcgctcagctccttgagatgcttagggatcagcttgctaagtggcagtgggagactcatgatccatgggtttgcgctccagatggtgtgcttgaagagaagttgtctccacagtgccagccacttcataacgagcttctcgagcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtactacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattatttcatcatcatcaccaccac

SEQ ID NO: 57 - protein, 766 aa, synthetic construct [sequence omitted]

SEQ ID NO: 58 - DNA, 2326 bp, synthetic construct
  source 1..2326: mol_type = other DNA
aacaatggcttcctccgctactactaaatctttccttattcttttctttatgatacttgcaactacttcatcaacatgtgcagtcgaccgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggaactctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgctcgagcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtactacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattatttcatcatcatcaccaccac

SEQ ID NO: 59 - protein, 774 aa, synthetic construct
MASSATTKSFLILFFMILATTSSTCAVDRPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELLEPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLFHHHHHH

SEQ ID NO: 60 - DNA, 2326 bp, synthetic construct
  source 1..2326: mol_type = other DNA
aacaatggcttcctccgctactactaaatctttccttattcttttctttatgatacttgcaactacttcatcaacatgtgcagtcgacaggcctaggaacgctcttttgcttcttgctgatgatggcggattcgagtccggtgcttacaacaactctgctatcgctactccacacctcgatgctcttgctaggcgttctcttcttttccgtaacgctttcacttccgtgtcctcttgctcaccttctagggcttcacttcttactggacttccacagcaccagaacggaatgtacggacttcatcaggatgtgcaccacttcaactcattcgataaggtgagatccctcccactcctcttgtctcaagctggtgttaggactggaatcatcggcaaaaagcacgtgggaccagagactgtgtacccattcgatttcgcttacactgaggaaaacggctccgttcttcaagtgggcagaaatattactaggatcaagctcctcgtgaggaagttcctccagactcaagatgataggccattcttcctctacgtggcattccatgatccacataggtgcggacattctcagccacagtacggaactttctgcgagaagttcggaaacggtgagtctggtatgggcaggattccagattggactccacaggcttacgatccacttgatgtgctcgttccatacttcgtgccaaacactccagctgctagagctgatcttgctgctcagtacactactgtgggaaggatggatcagggtgtgggacttgtgcttcaagagcttagagatgctggcgtgctcaacgatactctcgtgatcttcacttcagataacggcatcccattcccatccggaaggactaatctttactggccaggtactgctgagcctctccttgtttcttcaccagagcatccaaagaggtggggacaagtttctgaggcttacgtgtcccttctcgatctcactccaactatcctcgattggttctccatcccttacccatcctacgctatcttcggctccaagactatccaccttactggcagatctttgctcccagctttggaagctgaaccactttgggctactgtgttcggatctcagtctcaccacgaggtgacaatgtcttacccaatgagatctgtgcagcacaggcacttcaggcttgttcacaacctcaacttcaagatgccattcccaatcgatcaggatttctacgtgtcaccaactttccaggatcttctcaacaggactactgcaggacaacctactggctggtacaaggatcttaggcactactactatagggctaggtgggagctttacgataggtccagagatccacacgagactcagaaccttgctactgatccaaggttcgctcagctccttgagatgcttagggatcagcttgctaagtggcagtgggagactcatgatccatgggtttgcgctccagatggtgtgcttgaagagaagttgtctccacagtgccagccacttcataacgagcttctcgagcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtactacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattatttcatcatcatcaccaccac

SEQ ID NO: 61 - protein, 774 aa, synthetic construct; identical to SEQ ID NO: 59

SEQ ID NO: 62 - DNA, 2331 bp, synthetic construct
  source 1..2331: mol_type = other DNA
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggaactctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgctcgagggagaaacttctactcttaggacttcattcacaagaaacatcgttggtcgtgatggattgtgcgtggatgtgaggaatggatacgacactgatggaactccacttcagttgtggccatgtggaacccagagaaaccaacgatggacttttgactcagacgatacaatcaggtcaatgggtaaatgcatgactgcaaacgggttaaacaatggaagcaatatcgtgatattcaactgttctacagctgctgagaacgccattaagtgggaagtacctattgatggcagcatcatcaatccttcctccggattggttatgaccgctcctcgtgctgcatcccgtaccatcctgttgcttgaggacaatatctacgccgctagccagggttggactgtgacaaacaatgtaaagcccatcgttgcttccattgtgggttataaagaaatgtgcttgcagtctaacggtgaaaacaatggtgtttggatggaggattgtgaggccaccagtttgcagcaacagtgggcactctatggtgaccgtaccatccgagtaaatagtactcgtggcttatgcgtcaccaccaatgggtacaactccaaggatctcatcatcatccttaaatgccaaggattgcccagccagaggtggtttttcaactccgacggcgccatcgtaaacccaaagtcaagacatgttatggatgtgagagcaagcaatgtctctcttcgagaaatcattatctttccagccactgggaaccctaatcagcaatgggtgacacaagtccttccaagtcccgggcatcatcatcatcatcat

SEQ ID NO: 63 - protein, 777 aa, synthetic construct
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELLEGETSTLRTSFTRNIVGRDGLCVDVRNGYDTDGTPLQLWPCGTQRNQRWTFDSDDTIRSMGKCMTANGLNNGSNIVIFNCSTAAENAIKWEVPIDGSIINPSSGLVMTAPRAASRTILLLEDNIYAASQGWTVTNNVKPIVASIVGYKEMCLQSNGENNGVWMEDCEATSLQQQWALYGDRTIRVNSTRGLCVTTNGYNSKDLIIILKCQGLPSQRWFFNSDGAIVNPKSRHVMDVRASNVSLREIIIFPATGNPNQQWVTQVLPSPGHHHHHH

SEQ ID NO: 64 - DNA, 2331 bp, synthetic construct
  source 1..2331: mol_type = other DNA
atgtcttgtccagttccagcttgttgcgctctccttcttgttcttggattgtgtagggcaaggcctaggaacgctcttttgcttcttgctgatgatggcggattcgagtccggtgcttacaacaactctgctatcgctactccacacctcgatgctcttgctaggcgttctcttcttttccgtaacgctttcacttccgtgtcctcttgctcaccttctagggcttcacttcttactggacttccacagcaccagaacggaatgtacggacttcatcaggatgtgcaccacttcaactcattcgataaggtgagatccctcccactcctcttgtctcaagctggtgttaggactggaatcatcggcaaaaagcacgtgggaccagagactgtgtacccattcgatttcgcttacactgaggaaaacggctccgttcttcaagtgggcagaaatattactaggatcaagctcctcgtgaggaagttcctccagactcaagatgataggccattcttcctctacgtggcattccatgatccacataggtgcggacattctcagccacagtacggaactttctgcgagaagttcggaaacggtgagtctggtatgggcaggattccagattggactccacaggcttacgatccacttgatgtgctcgttccatacttcgtgccaaacactccagctgctagagctgatcttgctgctcagtacactactgtgggaaggatggatcagggtgtgggacttgtgcttcaagagcttagagatgctggcgtgctcaacgatactctcgtgatcttcacttcagataacggcatcccattcccatccggaaggactaatctttactggccaggtactgctgagcctctccttgtttcttcaccagagcatccaaagaggtggggacaagtttctgaggcttacgtgtcccttctcgatctcactccaactatcctcgattggttctccatcccttacccatcctacgctatcttcggctccaagactatccaccttactggcagatctttgctcccagctttggaagctgaaccactttgggctactgtgttcggatctcagtctcaccacgaggtgacaatgtcttacccaatgagatctgtgcagcacaggcacttcaggcttgttcacaacctcaacttcaagatgccattcccaatcgatcaggatttctacgtgtcaccaactttccaggatcttctcaacaggactactgcaggacaacctactggctggtacaaggatcttaggcactactactatagggctaggtgggagctttacgataggtccagagatccacacgagactcagaaccttgctactgatccaaggttcgctcagctccttgagatgcttagggatcagcttgctaagtggcagtgggagactcatgatccatgggtttgcgctccagatggtgtgcttgaagagaagttgtctccacagtgccagccacttcataacgagcttctcgagggagaaacttctactcttaggacttcattcacaagaaacatcgttggtcgtgatggattgtgcgtggatgtgaggaatggatacgacactgatggaactccacttcagttgtggccatgtggaacccagagaaaccaacgatggacttttgactcagacgatacaatcaggtcaatgggtaaatgcatgactgcaaacgggttaaacaatggaagcaatatcgtgatattcaactgttctacagctgctgagaacgccattaagtgggaagtacctattgatggcagcatcatcaatccttcctccggattggttatgaccgctcctcgtgctgcatcccgtaccatcctgttgcttgaggacaatatctacgccgctagccagggttggactgtgacaaacaatgtaaagcccatcgttgcttccattgtgggttataaagaaatgtgcttgcagtctaacggtgaaaacaatggtgtttggatggaggattgtgaggccaccagtttgcagcaacagtgggcactctatggtgaccgtaccatccgagtaaatagtactcgtggcttatgcgtcaccaccaatgggtacaactccaaggatctcatcatcatccttaaatgccaaggattgcccagccagaggtggtttttcaactccgacggcgccatcgtaaacccaaagtcaagacatgttatggatgtgagagcaagcaatgtctctcttcgagaaatcattatctttccagccactgggaaccctaatcagcaatgggtgacacaagtccttccaagtcccgggcatcatcatcatcatcat

SEQ ID NO: 65 - protein, 777 aa, synthetic construct; identical to SEQ ID NO: 63

SEQ ID NO: 66 - DNA, 2359 bp, synthetic construct
  source 1..2359: mol_type = other DNA
aacaatggcttcctccgctactactaaatctttccttattcttttctttatgatacttgcaactacttcatcaacatgtgcagtcgaccgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggaactctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgctcgagggagaaacttctactcttaggacttcattcacaagaaacatcgttggtcgtgatggattgtgcgtggatgtgaggaatggatacgacactgatggaactccacttcagttgtggccatgtggaacccagagaaaccaacgatggacttttgactcagacgatacaatcaggtcaatgggtaaatgcatgactgcaaacgggttaaacaatggaagcaatatcgtgatattcaactgttctacagctgctgagaacgccattaagtgggaagtacctattgatggcagcatcatcaatccttcctccggattggttatgaccgctcctcgtgctgcatcccgtaccatcctgttgcttgaggacaatatctacgccgctagccagggttggactgtgacaaacaatgtaaagcccatcgttgcttccattgtgggttataaagaaatgtgcttgcagtctaacggtgaaaacaatggtgtttggatggaggattgtgaggccaccagtttgcagcaacagtgggcactctatggtgaccgtaccatccgagtaaatagtactcgtggcttatgcgtcaccaccaatgggtacaactccaaggatctcatcatcatccttaaatgccaaggattgcccagccagaggtggtttttcaactccgacggcgccatcgtaaacccaaagtcaagacatgttatggatgtgagagcaagcaatgtctctcttcgagaaatcattatctttccagccactgggaaccctaatcagcaatgggtgacacaagtccttccaagtcccgggcatcatcatcatcatcat

SEQ ID NO: 67 - protein, 785 aa, synthetic construct
MASSATTKSFLILFFMILATTSSTCAVDRPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELLEGETSTLRTSFTRNIVGRDGLCVDVRNGYDTDGTPLQLWPCGTQRNQRWTFDSDDTIRSMGKCMTANGLNNGSNIVIFNCSTAAENAIKWEVPIDGSIINPSSGLVMTAPRAASRTILLLEDNIYAASQGWTVTNNVKPIVASIVGYKEMCLQSNGENNGVWMEDCEATSLQQQWALYGDRTIRVNSTRGLCVTTNGYNSKDLIIILKCQGLPSQRWFFNSDGAIVNPKSRHVMDVRASNVSLREIIIFPATGNPNQQWVTQVLPSPGHHHHHH

SEQ ID NO: 68 - DNA, 2359 bp, synthetic construct
  source 1..2359: mol_type = other DNA
aacaatggcttcctccgctactactaaatctttccttattcttttctttatgatacttgcaactacttcatcaacatgtgcagtcgacaggcctaggaacgctcttttgcttcttgctgatgatggcggattcgagtccggtgcttacaacaactctgctatcgctactccacacctcgatgctcttgctaggcgttctcttcttttccgtaacgctttcacttccgtgtcctcttgctcaccttctagggcttcacttcttactggacttccacagcaccagaacggaatgtacggacttcatcaggatgtgcaccacttcaactcattcgataaggtgagatccctcccactcctcttgtctcaagctggtgttaggactggaatcatcggcaaaaagcacgtgggaccagagactgtgtacccattcgatttcgcttacactgaggaaaacggctccgttcttcaagtgggcagaaatattactaggatcaagctcctcgtgaggaagttcctccagactcaagatgataggccattcttcctctacgtggcattccatgatccacataggtgcggacattctcagccacagtacggaactttctgcgagaagttcggaaacggtgagtctggtatgggcaggattccagattggactccacaggcttacgatccacttgatgtgctcgttccatacttcgtgccaaacactccagctgctagagctgatcttgctgctcagtacactactgtgggaaggatggatcagggtgtgggacttgtgcttcaagagcttagagatgctggcgtgctcaacgatactctcgtgatcttcacttcagataacggcatcccattcccatccggaaggactaatctttactggccaggtactgctgagcctctccttgtttcttcaccagagcatccaaagaggtggggacaagtttctgaggcttacgtgtcccttctcgatctcactccaactatcctcgattggttctccatcccttacccatcctacgctatcttcggctccaagactatccaccttactggcagatctttgctcccagctttggaagctgaaccactttgggctactgtgttcggatctcagtctcaccacgaggtgacaatgtcttacccaatgagatctgtgcagcacaggcacttcaggcttgttcacaacctcaacttcaagatgccattcccaatcgatcaggatttctacgtgtcaccaactttccaggatcttctcaacaggactactgcaggacaacctactggctggtacaaggatcttaggcactactactatagggctaggtgggagctttacgataggtccagagatccacacgagactcagaaccttgctactgatccaaggttcgctcagctccttgagatgcttagggatcagcttgctaagtggcagtgggagactcatgatccatgggtttgcgctccagatggtgtgcttgaagagaagttgtctccacagtgccagccacttcataacgagcttctcgagggagaaacttctactcttaggacttcattcacaagaaacatcgttggtcgtgatggattgtgcgtggatgtgaggaatggatacgacactgatggaactccacttcagttgtggccatgtggaacccagagaaaccaacgatggacttttgactcagacgatacaatcaggtcaatgggtaaatgcatgactgcaaacgggttaaacaatggaagcaatatcgtgatattcaactgttctacagctgctgagaacgccattaagtgggaagtacctattgatggcagcatcatcaatccttcctccggattggttatgaccgctcctcgtgctgcatcccgtaccatcctgttgcttgaggacaatatctacgccgctagccagggttggactgtgacaaacaatgtaaagcccatcgttgcttccattgtgggttataaagaaatgtgcttgcagtctaacggtgaaaacaatggtgtttggatggaggattgtgaggccaccagtttgcagcaacagtgggcactctatggtgaccgtaccatccgagtaaatagtactcgtggcttatgcgtcaccaccaatgggtacaactccaaggatctcatcatcatccttaaatgccaaggattgcccagccagaggtggtttttcaactccgacggcgccatcgtaaacccaaagtcaagacatgttatggatgtgagagcaagcaatgtctctcttcgagaaatcattatctttccagccactgggaaccctaatcagcaatgggtgacacaagtccttccaagtcccgggcatcatcatcatcatcat

SEQ ID NO: 69 - protein, 785 aa, synthetic construct; identical to SEQ ID NO: 67

SEQ ID NO: 70 - DNA, 1140 bp, synthetic construct
  source 1..1140: mol_type = other DNA
atggctgcgcccgcactagggctggtgtgtggacgttgccctgagctgggtctcgtcctcttgctgctgctgctctcgctgctgtgtggagcggcagggagccaggaggccgggaccggtgcgggcgcggggtcccttgcgggttcttgcggctgcggcacgccccagcggcctggcgcccatggcagttcggcagccgctcaccgatactcgcgggaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccactatggaccatcatcatcatcatcat

SEQ ID NO: 71 - protein, 380 aa, synthetic construct
MAAPALGLVCGRCPELGLVLLLLLLSLLCGAAGSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDHHHHHH

SEQ ID NO: 72 - DNA, 1140 bp, synthetic construct
  source 1..1140: mol_type = other DNA
atggctgctccagctcttggacttgtttgtggaagatgtccagagcttggactcgtgctccttttgcttcttttgtcacttctctgcggagctgctggatctcaagaggctggaactggtgctggtgctggatctcttgctggatcttgtggatgtggaactccacaaagaccaggtgctcatggatcttcagctgctgctcataggtactctagggaagctaatgctccaggtccagttccaggtgagagacagcttgctcactctaagatggtgccaattccagctggcgtgttcactatgggaactgatgatccacagatcaagcaggatggtgaggctccagctagaagggtgacaatcgatgctttctacatggatgcttacgaggtgtccaacactgagttcgagaagttcgtgaactccactggctaccttactgaggctgagaagttcggcgattccttcgttttcgagggaatgctctctgagcaggttaagactaacatccagcaggctgttgctgctgctccatggtggcttccagttaagggtgctaattggagacatccagagggcccagattccactattcttcataggccagatcacccagtgctccacgtttcatggaatgatgctgtggcttactgcacttgggctggaaagagacttccaactgaagctgagtgggagtactcttgtaggggaggacttcacaacaggcttttcccatggggaaacaagttgcagccaaagggacagcactacgctaatatttggcaaggcgagttcccagtgactaacactggtgaggatggattccaaggtactgctccagttgatgctttcccaccaaatggatacggcctctacaacatcgttggaaacgcttgggagtggacttccgattggtggactgttcatcactccgtggaagagactctcaacccaaagggaccaccatctggaaaggatagggttaagaaaggcggctcctacatgtgccataggtcttactgttacaggtacaggtgcgctgctaggtcccagaatactccagattcctctgcttccaacctcggattcagatgtgctgctgataggctcccaactatggatcatcatcaccatcaccac

SEQ ID NO: 73 - protein, 380 aa, synthetic construct; identical to SEQ ID NO: 71

SEQ ID NO: 74 - DNA, 1129 bp, synthetic construct
  source 1..1129: mol_type = other DNA
aacaatggcttcctccgctactactaaatctttccttattcttttctttatgatacttgcaactacttcatcaacatgtgcagtcgacagccaggaggccgggaccggtgcgggcgcggggtcccttgcgggttcttgcggctgcggcacgccccagcggcctggcgcccatggcagttcggcagccgctcaccgatactcgcgggaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccactatggaccatcatcatcatcatcat

SEQ ID NO: 75 - protein, 375 aa, synthetic construct
MASSATTKSFLILFFMILATTSSTCAVDSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDHHHHHH

SEQ ID NO: 76 - DNA, 1129 bp, synthetic construct
  source 1..1129: mol_type = other DNA
aacaatggcttcctccgctactactaaatctttccttattcttttctttatgatacttgcaactacttcatcaacatgtgcagtcgactctcaagaggctggaactggtgctggtgctggatctcttgctggatcttgtggatgtggaactccacaaagaccaggtgctcatggatcttcagctgctgctcataggtactctagggaagctaatgctccaggtccagttccaggtgagagacagcttgctcactctaagatggtgccaattccagctggcgtgttcactatgggaactgatgatccacagatcaagcaggatggtgaggctccagctagaagggtgacaatcgatgctttctacatggatgcttacgaggtgtccaacactgagttcgagaagttcgtgaactccactggctaccttactgaggctgagaagttcggcgattccttcgttttcgagggaatgctctctgagcaggttaagactaacatccagcaggctgttgctgctgctccatggtggcttccagttaagggtgctaattggagacatccagagggcccagattccactattcttcataggccagatcacccagtgctccacgtttcatggaatgatgctgtggcttactgcacttgggctggaaagagacttccaactgaagctgagtgggagtactcttgtaggggaggacttcacaacaggcttttcccatggggaaacaagttgcagccaaagggacagcactacgctaatatttggcaaggcgagttcccagtgactaacactggtgaggatggattccaaggtactgctccagttgatgctttcccaccaaatggatacggcctctacaacatcgttggaaacgcttgggagtggacttccgattggtggactgttcatcactccgtggaagagactctcaacccaaagggaccaccatctggaaaggatagggttaagaaaggcggctcctacatgtgccataggtcttactgttacaggtacaggtgcgctgctaggtcccagaatactccagattcctctgcttccaacctcggattcagatgtgctgctgataggctcccaactatggatcatcatcaccatcaccac

SEQ ID NO: 77 - protein, 375 aa, synthetic construct; identical to SEQ ID NO: 75

SEQ ID NO: 78 - DNA, 1141 bp, synthetic construct
  source 1..1141: mol_type = other DNA
aacaatggcttcctccgctactactaaatctttccttattcttttctttatgatacttgcaactacttcatcaacatgtgcagtcgacagccaggaggccgggaccggtgcgggcgcggggtcccttgcgggttcttgcggctgcggcacgccccagcggcctggcgcccatggcagttcggcagccgctcaccgatactcgcgggaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccactatggaccatcatcatcatcatcataaggatgaactt

SEQ ID NO: 79 - protein, 379 aa, synthetic construct
MASSATTKSFLILFFMILATTSSTCAVDSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDHHHHHHKDEL

SEQ ID NO: 80 - DNA, 1141 bp, synthetic construct
  source 1..1141: mol_type = other DNA
aacaatggcttcctccgctactactaaatctttccttattcttttctttatgatacttgcaactacttcatcaacatgtgcagtcgactctcaagaggctggaactggtgctggtgctggatctcttgctggatcttgtggatgtggaactccacaaagaccaggtgctcatggatcttcagctgctgctcataggtactctagggaagctaatgctccaggtccagttccaggtgagagacagcttgctcactctaagatggtgccaattccagctggcgtgttcactatgggaactgatgatccacagatcaagcaggatggtgaggctccagctagaagggtgacaatcgatgctttctacatggatgcttacgaggtgtccaacactgagttcgagaagttcgtgaactccactggctaccttactgaggctgagaagttcggcgattccttcgttttcgagggaatgctctctgagcaggttaagactaacatccagcaggctgttgctgctgctccatggtggcttccagttaagggtgctaattggagacatccagagggcccagattccactattcttcataggccagatcacccagtgctccacgtttcatggaatgatgctgtggcttactgcacttgggctggaaagagacttccaactgaagctgagtgggagtactcttgtaggggaggacttcacaacaggcttttcccatggggaaacaagttgcagccaaagggacagcactacgctaatatttggcaaggcgagttcccagtgactaacactggtgaggatggattccaaggtactgctccagttgatgctttcccaccaaatggatacggcctctacaacatcgttggaaacgcttgggagtggacttccgattggtggactgttcatcactccgtggaagagactctcaacccaaagggaccaccatctggaaaggatagggttaagaaaggcggctcctacatgtgccataggtcttactgttacaggtacaggtgcgctgctaggtcccagaatactccagattcctctgcttccaacctcggattcagatgtgctgctgataggctcccaactatggatcatcatcaccatcaccacaaggatgaactt

SEQ ID NO: 81 - protein, 379 aa, synthetic construct; identical to SEQ ID NO: 79

SEQ ID NO: 82 - protein, 4 aa, Homo sapiens
KDEL